(19) United States
(12) Patent Application Publication
(10) Pub. No.: US 2023/0320198 A1
(43) Pub. Date: Oct. 5, 2023

(54) COMPOUND AND APPLICATION THEREOF

(71) Applicant: GUANGDONG AGLAIA OPTOELECTRONIC MATERIALS CO., LTD, Foshan, Guangdong (CN)

(72) Inventors: Liangliang YAN, Foshan (CN); Shaofu CHEN, Foshan (CN); Lei DAI, Foshan (CN); Lifei CAI, Foshan (CN)

(73) Assignee: GUANGDONG AGLAIA OPTOELECTRONIC MATERIALS CO., LTD, Foshan, Guangdong (CN)

(21) Appl. No.: 17/926,900

(22) PCT Filed: May 9, 2021
(86) PCT No.: PCT/CN2021/092521

§ 371 Date: Nov. 21, 2022

(30) Foreign Application Priority Data
May 25, 2020 (CN) 202010447388.5

(51) Int. Cl. H10K 85/60; C07C 211/54; C07D 307/91; C09K 11/06; C07D 209/86; C07C 211/61

(52) U.S. Cl. CPC H10K 85/633; C07C 211/54; C07D 307/91; C09K 11/06; H10K 85/636; C07D 209/86; C07C 211/61; C07C 2603/40; C07C 2603/26; C07C 2603/97; C07C 2603/18; H10K 50/156

(57) ABSTRACT

Provided in the present invention are a compound and application thereof. The compound of the present invention has a structure as shown in the formula (1). The compound obtained in the present invention by connecting phenanthrene and fluoranthene groups via nitrogen has the advantages of great optical, electrical, and thermal stability, high luminescence efficiency, low electric voltage, and long service life, and can be used in organic electroluminescent devices. In particular, the compound has the potential for application in the AMOLED industry as an electron blocking layer material or a hole transport layer material.

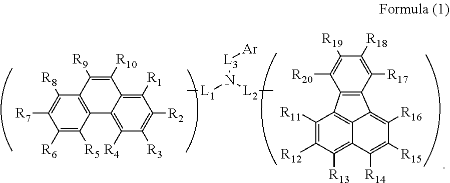

### TECHNICAL FIELD

The present invention relates to the technical field of organic electroluminescence, in particular to an organic light-emitting material applicable to organic electroluminescent devices, and specially in particular to a compound obtained by connecting phenanthrene and fluoranthene groups via nitrogen and application thereof in an organic electroluminescent device.

### BACKGROUND

At present, as a new-generation display technology, an organic electroluminescent device (OLED) has attracted more and more attention in display and lighting technologies, thus having a wide application prospect. However, compared with market application requirements, properties, such as luminescence efficiency, driving voltage, and service life of OLED devices still need to be strengthened and improved.

In generally, the OLED devices include various organic functional material films with different functions between metal electrodes as basic structures, which are similar to sandwich structures. Under the driving of a current, holes and electrons are injected from a cathode and an anode, respectively. After moving a certain distance, the holes and the electrons are compounded in a light-emitting layer, and then released in the form of light or heat to achieve luminescence of the OLED. However, organic functional materials are core components of the OLED devices, and the thermal stability, photochemical stability, electrochemical stability, quantum yield, film forming stability, crystallinity, and color saturation of the materials are main factors affecting properties of the devices.

According to a patent document 1 (US20150155491), compounds obtained by bonding 3-phenanthryl to nitrogen atoms directly or via connectors are described. The compounds can be used as hole injection layer materials, hole transport layer materials, electron blocking layer materials and the like in the OLED devices. According to a patent document 2 (JP2014511352), compounds obtained by bonding 2-phenanthryl to nitrogen atoms directly or via connectors are described. The compounds are used as hole transport layer materials or electron blocking layer materials in the OLED devices. According to a patent document 3 (CN107848950), compounds obtained by bonding two phenanthryls to nitrogen atoms directly or via connectors are described. The compounds are used as electron blocking layer materials in the OLED devices, and properties, such as optical, electrical, and thermal stability and luminescence efficiency, of the compounds still need to be further improved.

### SUMMARY

In order to solve the above problems, objectives of the present invention are to provide an organic electroluminescent device with high properties and to provide a novel material capable of realizing the organic electroluminescent device.

In order to achieve the above objectives, the inventor has conducted in-depth studies repeatedly and found that an organic electroluminescent device with high properties can be obtained by using a compound as shown in the following formula (1).

One of the objectives of the present invention is to provide a compound obtained by connecting phenanthrene and fluoranthene via nitrogen. The compound has the advantages of good film forming property, great optical, electrical, and thermal stability, high luminescence efficiency, low electric voltage, and long service life, and can be used in organic electroluminescent devices. In particular, the compound has the potential for application in the AMOLED industry as an electron hole transport material or an electron blocking layer material.

In order to achieve the above objective, the present invention adopts the following technical solutions.

A compound has a structural formula as shown in the following formula (1):

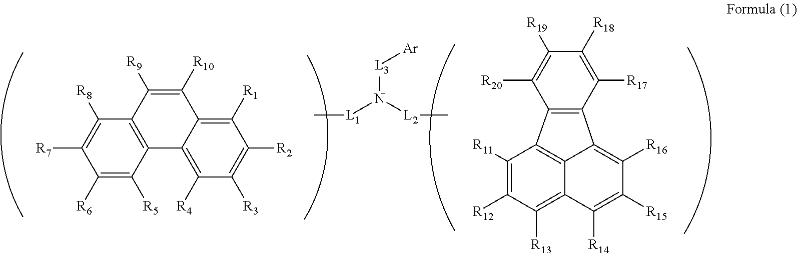

where any one of R₁-R₁₀ is a single bond for being bonded to L₁, and the other groups are substituents independently;
any one of R₁₁-R₂₀ is a single bond for being bonded to L₂, and the other groups are substituents independently;
each of the substituents is independently selected from hydrogen, deuterium, halogen, C₁-C₁₀ alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro; or two adjacent substituents are connected into a ring;
each of L₁-L₃ independently refers to a single bond, arylene with a ring forming carbon number of C₆₋₅₀ unsubstituted or substituted with R, and heteroarylene with a ring forming atom number of C₅₋₅₀ unsubstituted or substituted with R;
Ar refers to aryl with a ring forming carbon number of 6-50 unsubstituted or substituted with R, heteroaryl with a ring forming atom number of 5-50 unsubstituted or substituted with R, and a monocyclic or polycyclic C₃-C₆₀ alicyclic ring or aromatic ring unsubstituted or substituted with R; or one or more of carbon atoms in the monocyclic or polycyclic C₃-C₆₀ alicyclic ring or aromatic ring unsubstituted or substituted with R are substituted with at least one heteroatom selected from O, S, N, Se, Si, and Ge; a heteroatom in the heteroaryl or heteroalkyl is at least one heteroatom selected from O, S, N, Se, Si, and Ge;
and the R is independently selected from deuterium, F, Cl, Br, C₁-C₄ alkyl, C₁-C₄alkoxy, C₃-C₂₀cycloalkyl, C₆-C₁₀ aryl, aralkyl with a carbon number of 7-30 of aryl with a ring forming carbon number of 6-10, alkoxy with a carbon number of 1-20, aryloxy with a ring forming carbon number of 6-10, and at least one group of monosubstituted, disubstituted or trisubstituted silyl, cyano and nitro with substituents selected from alkyl with a carbon number of 1-10 and aryl with a ring forming carbon number of 6-10.

Preferred compounds are as shown in the following formulas (1-1a) to (1-1d):

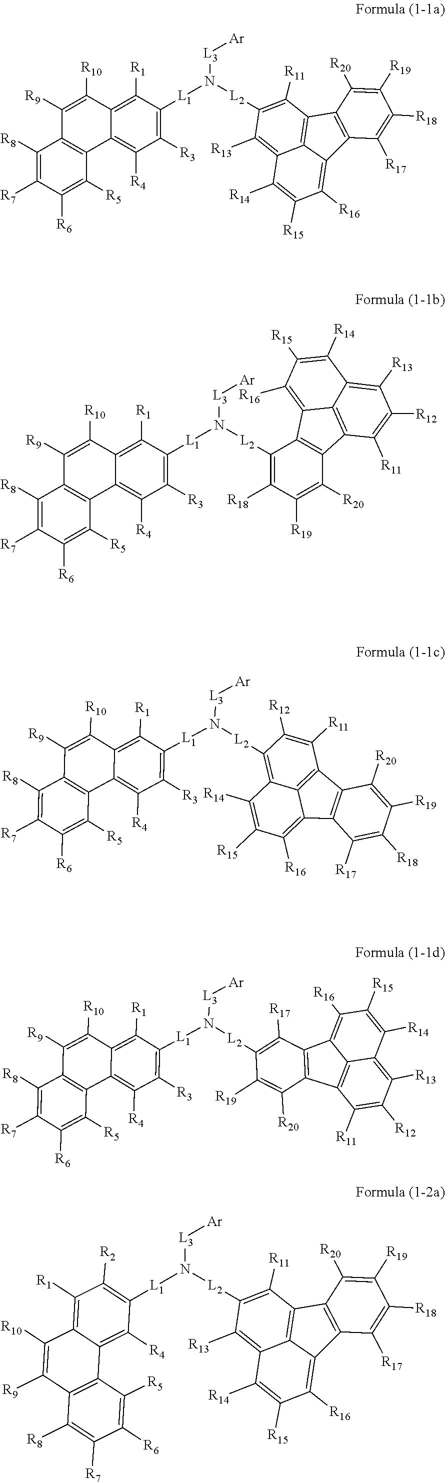

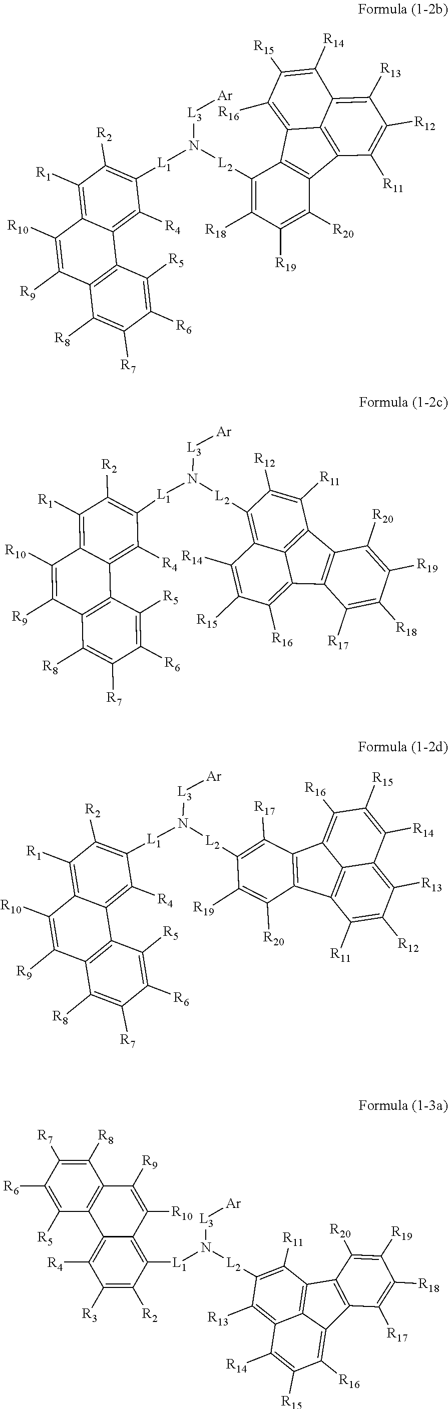

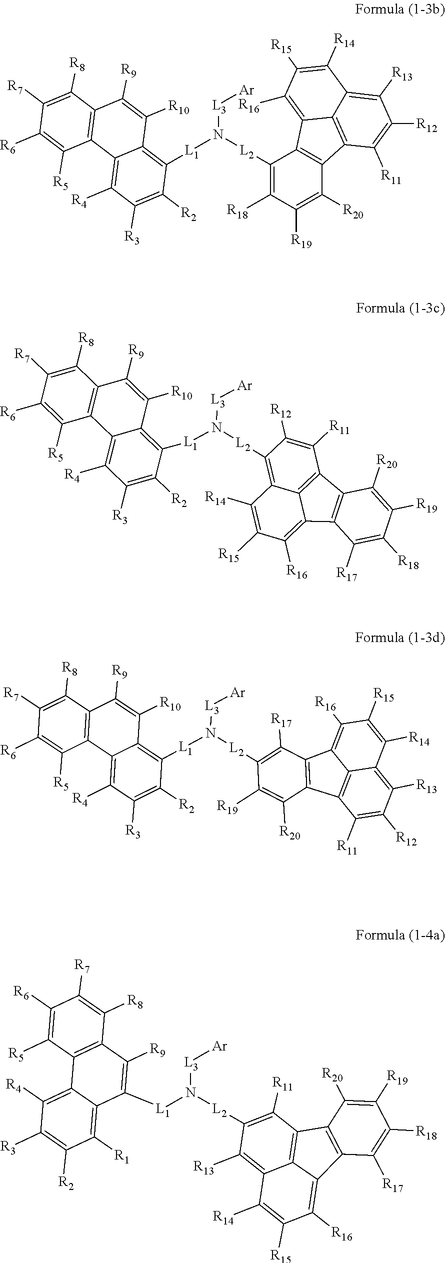

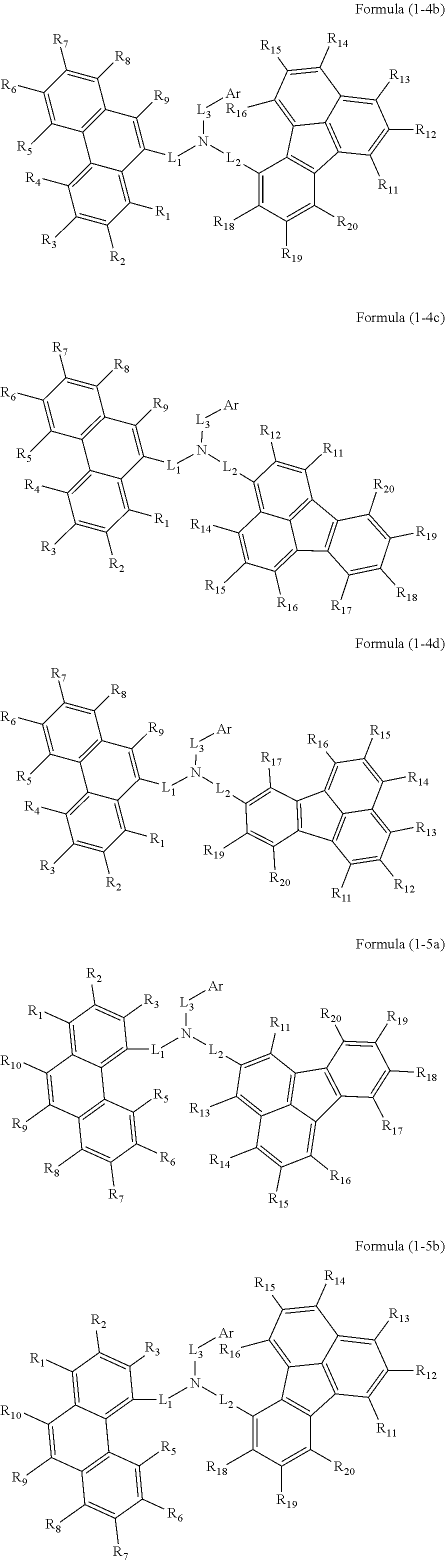

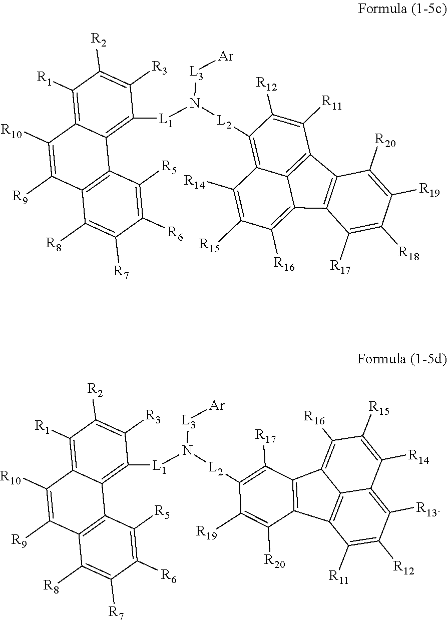

Preferably, each of the substituents, namely the R₁-R₂₀, is independently selected from hydrogen, deuterium, halogen, C₁-C₄ alkyl, C₃-C₆cycloalkyl, C₆-C₁₄aralkyl, C₁-C₁₄alkoxy, C₆-C₁₄aryloxy, amino, C₆-C₁₄ aryl, cyano, and nitro;
each of the L₁-L₃ independently refers to a single bond, arylene with a ring forming carbon number of 6-14 unsubstituted or substituted with R, and heteroarylene with a ring forming atom number of 5-13 unsubstituted or substituted with R;
the heteroatom in the heteroaryl is at least one heteroatom selected from O, S, and N;
and the R is independently selected from deuterium, F, Cl, Br, and C alkyl.

Further preferably, each of the R₁-R₄ and R₉-R₂₀ is independently selected from hydrogen; among the R₅-R₈, three groups are hydrogen, and the other group is hydrogen, C₁-C₄ alkyl, phenyl substituted with C₁-C₄ alkyl, phenyl, or naphthyl;
and each of the L₁-L₃ independently refers to a single bond, phenylene unsubstituted or substituted with C₁-C₄ alkyl, and naphthylene unsubstituted or substituted with C₁-C₄ alkyl.

The preferred compounds are characterized in that among the R₁-R₂₀, two adjacent substituents may be connected into a ring with a ring fused structure as shown in the following formula (2) or (3):

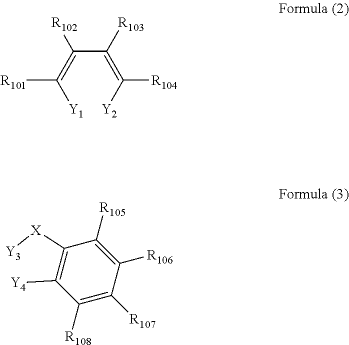

where Y₁, Y₂, Y₃, and Y₄ refer to positions connected to a ring; X is independently selected from O, S, SO₂, NR₁₀₉, CR₁₁₀R₁₁₁, and Si R₁₁₂R₁₁₃; and each of R₁₀₁-R₁₀₉ is independently selected from hydrogen, deuterium, halogen, C₁-C₁₀ alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro.

According to the preferred compounds, the Ar is as shown in any one of the following formulas (a) to (x):

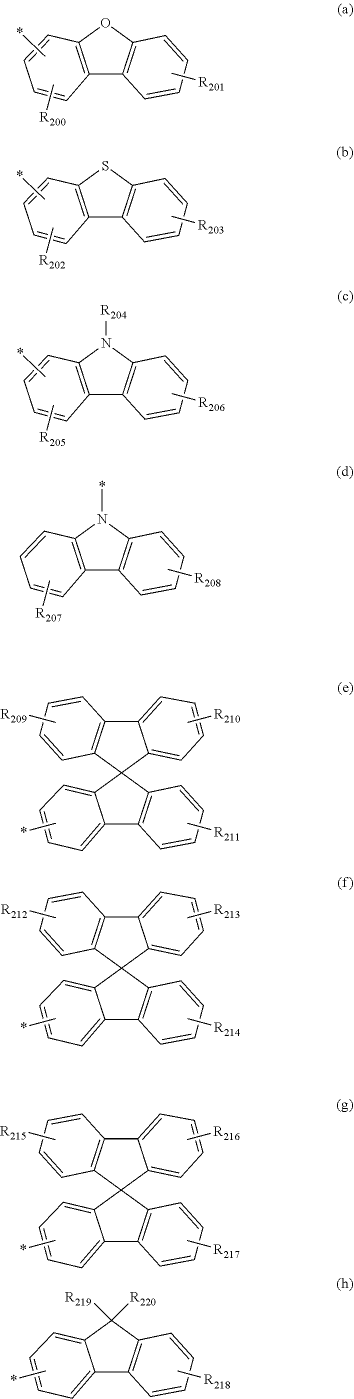

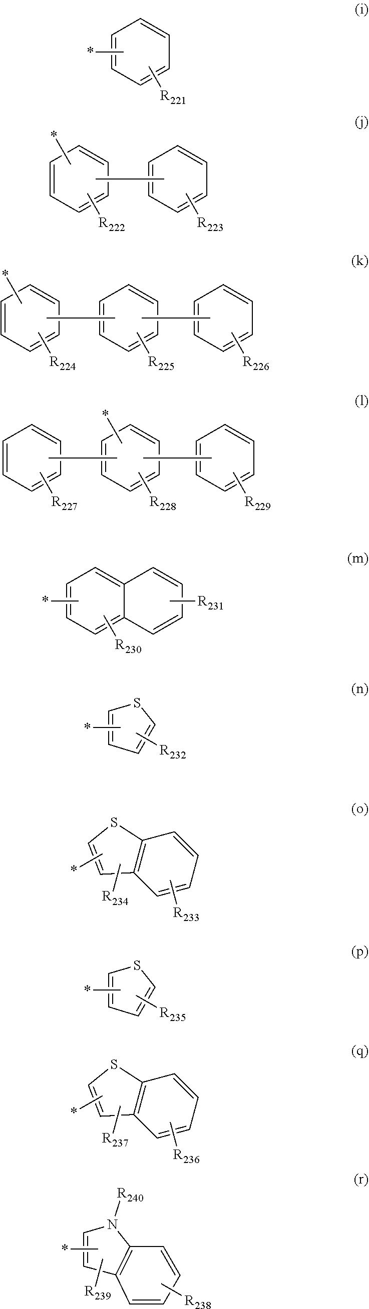

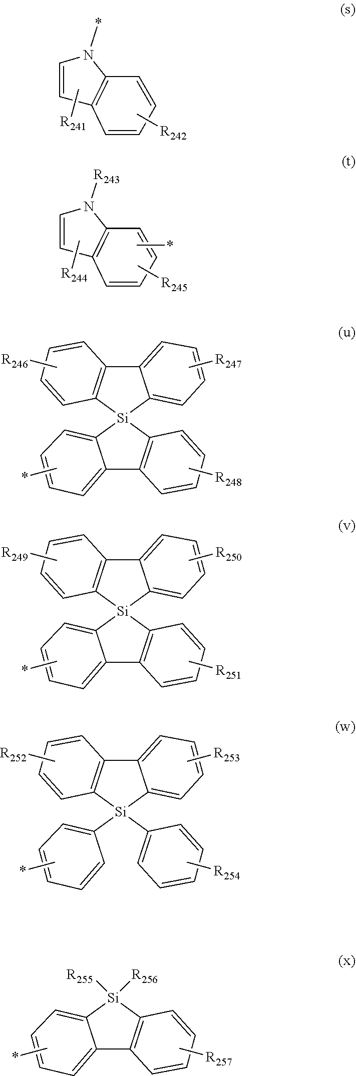

where each of R₂₀₀-R₂₅₇ independently refers to no substitution to a maximum possible substituent number; when the R₂₀₀-R₂₅₇ are substituents, each of the R₂₀₀-R₂₅₇ is independently selected from deuterium, halogen, C₁-C₁₀alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro; or two adjacent groups are connected into a ring;
and * refers to a bonding position connected to the L₃ in the formula (1).

Preferably, each of the R₂₀₀-R₂₅₇ is independently selected from hydrogen, C₁-C₄ alkyl, phenyl unsubstituted or substituted with C₁-C₄ alkyl, and naphthyl unsubstituted or substituted with C₁-C₄ alkyl.

As preferred compounds, the compounds specifically have the following structural formulas.

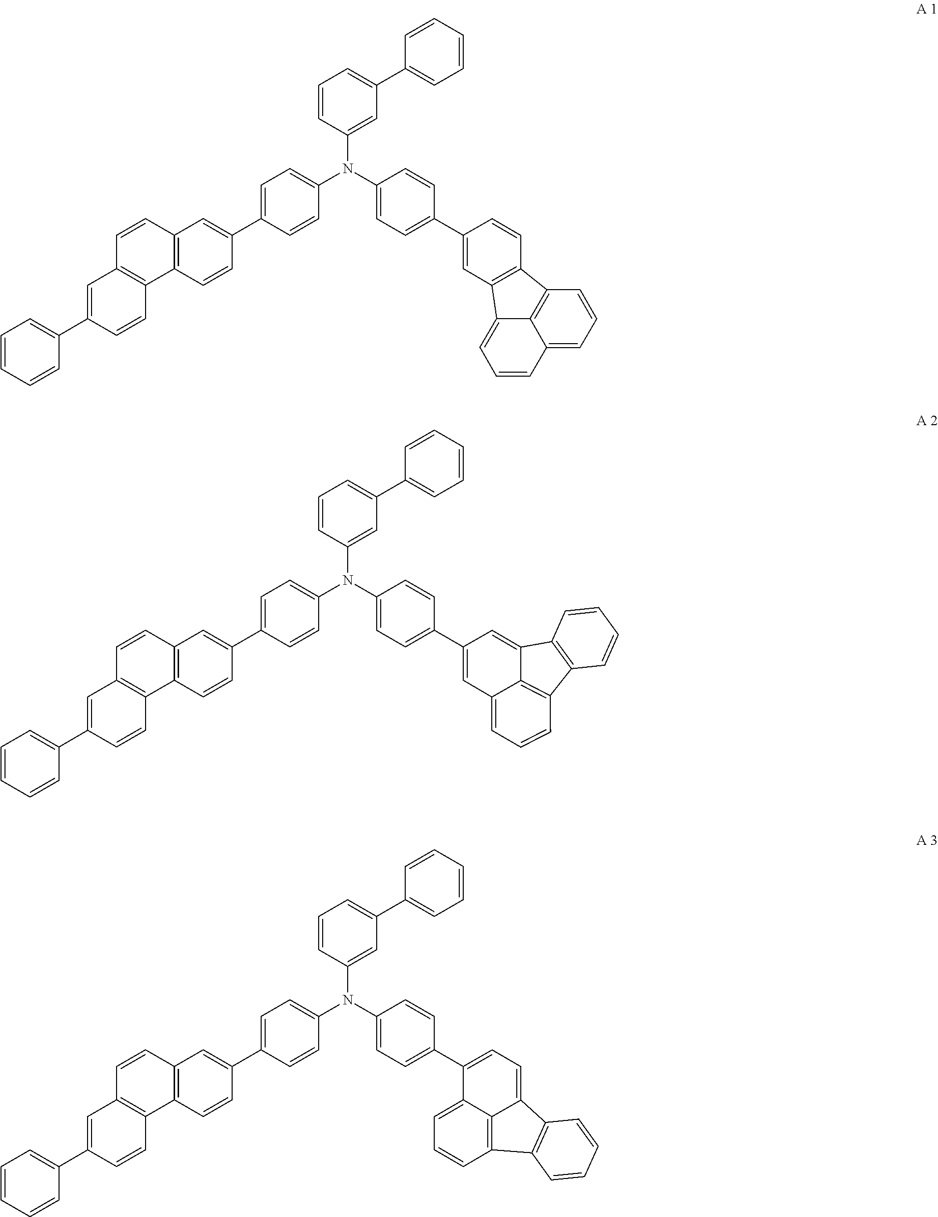 [SMILES: c1ccc(-c2cccc(N(c3ccc(-c4ccc5c(c4)-c4cccc6cccc-5c46)cc3)c3ccc(-c4ccc5c(ccc6cc(-c7ccccc7)ccc65)c4)cc3)c2)cc1 ; c1ccc(-c2cccc(N(c3ccc(-c4cc5c6c(cccc6c4)-c4ccccc4-5)cc3)c3ccc(-c4ccc5c(ccc6cc(-c7ccccc7)ccc65)c4)cc3)c2)cc1 ; c1ccc(-c2cccc(N(c3ccc(-c4ccc5c(ccc6cc(-c7ccccc7)ccc65)c4)cc3)c3ccc(-c4ccc5c6c(cccc46)-c4ccccc4-5)cc3)c2)cc1]

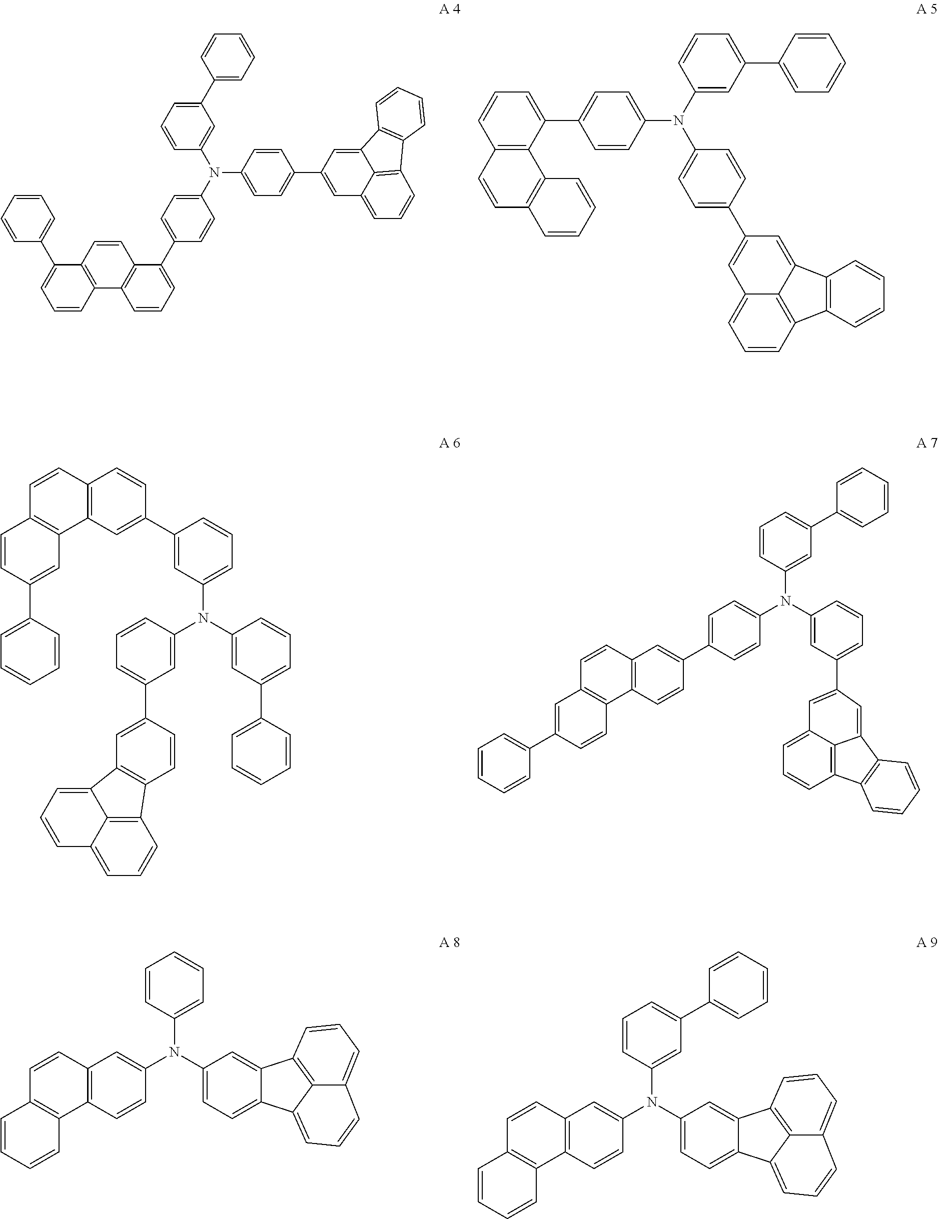

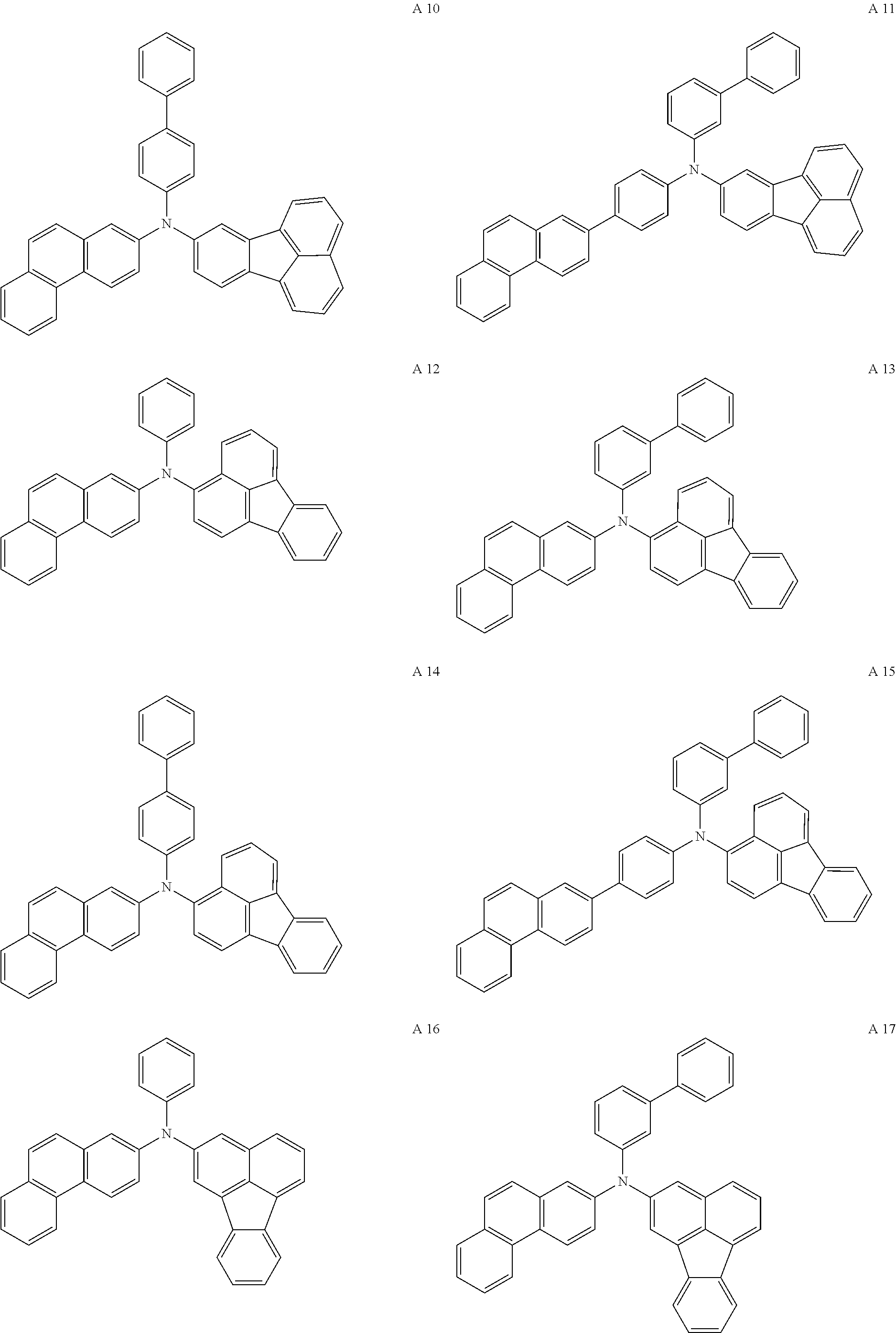

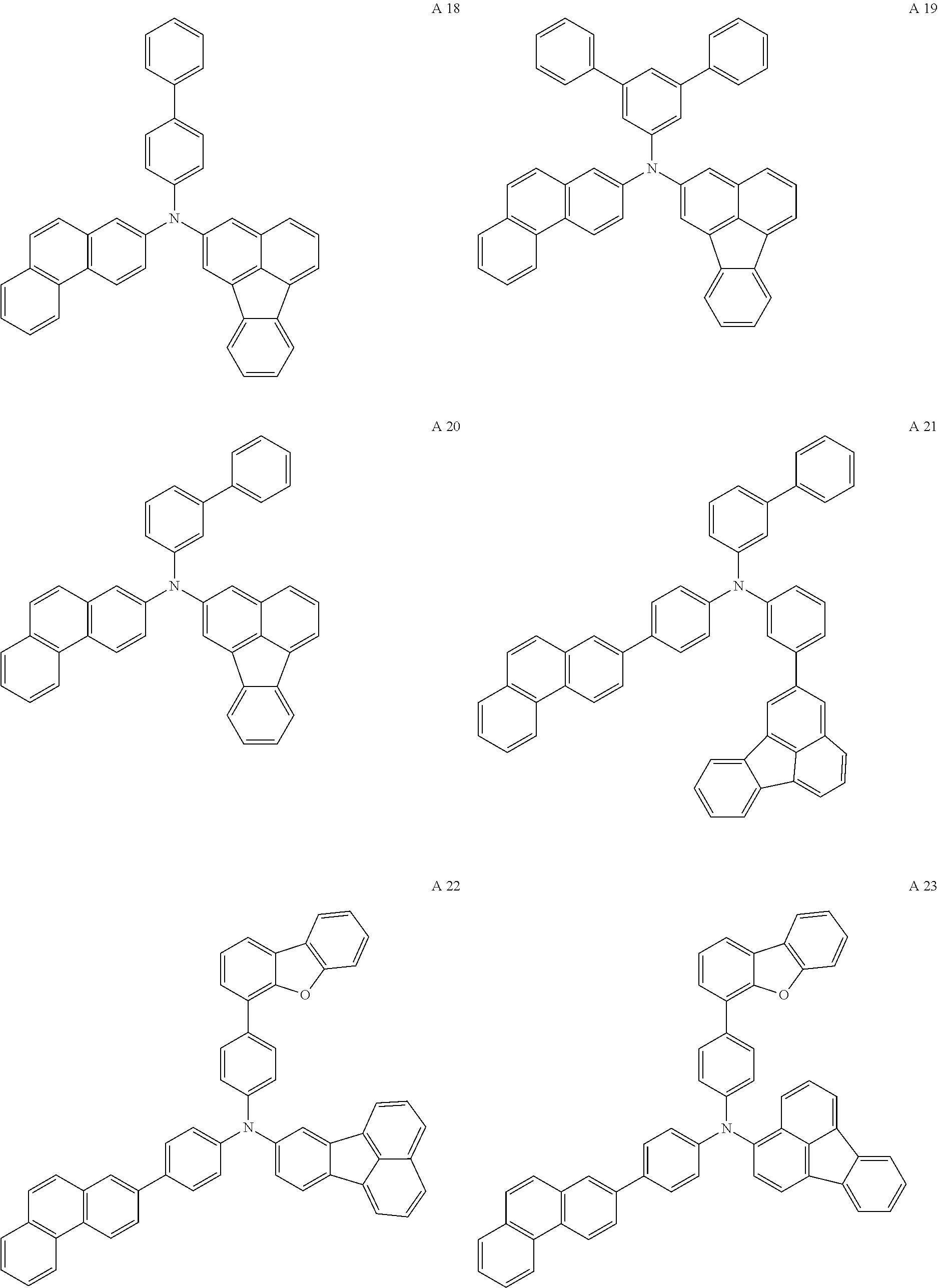

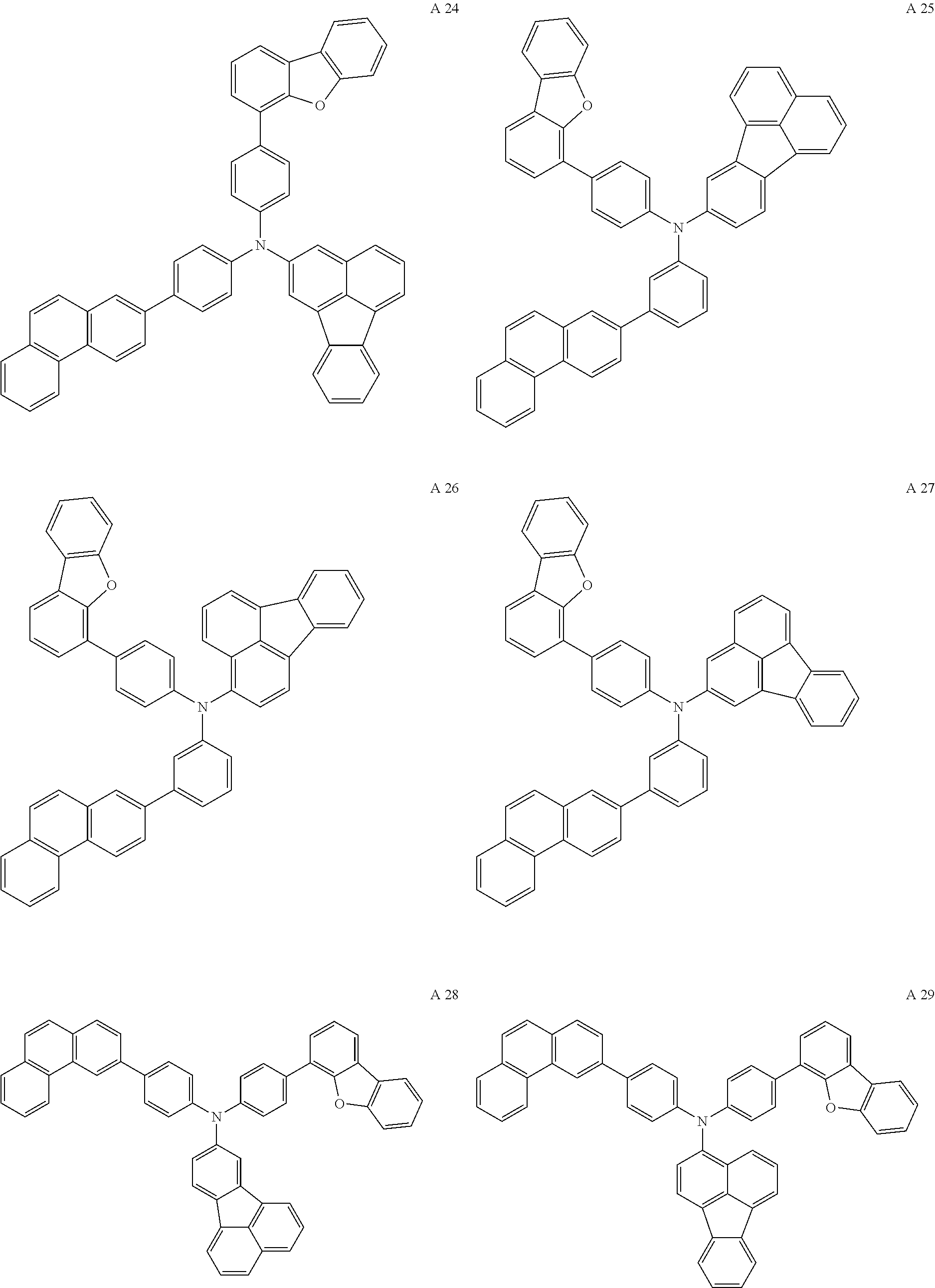

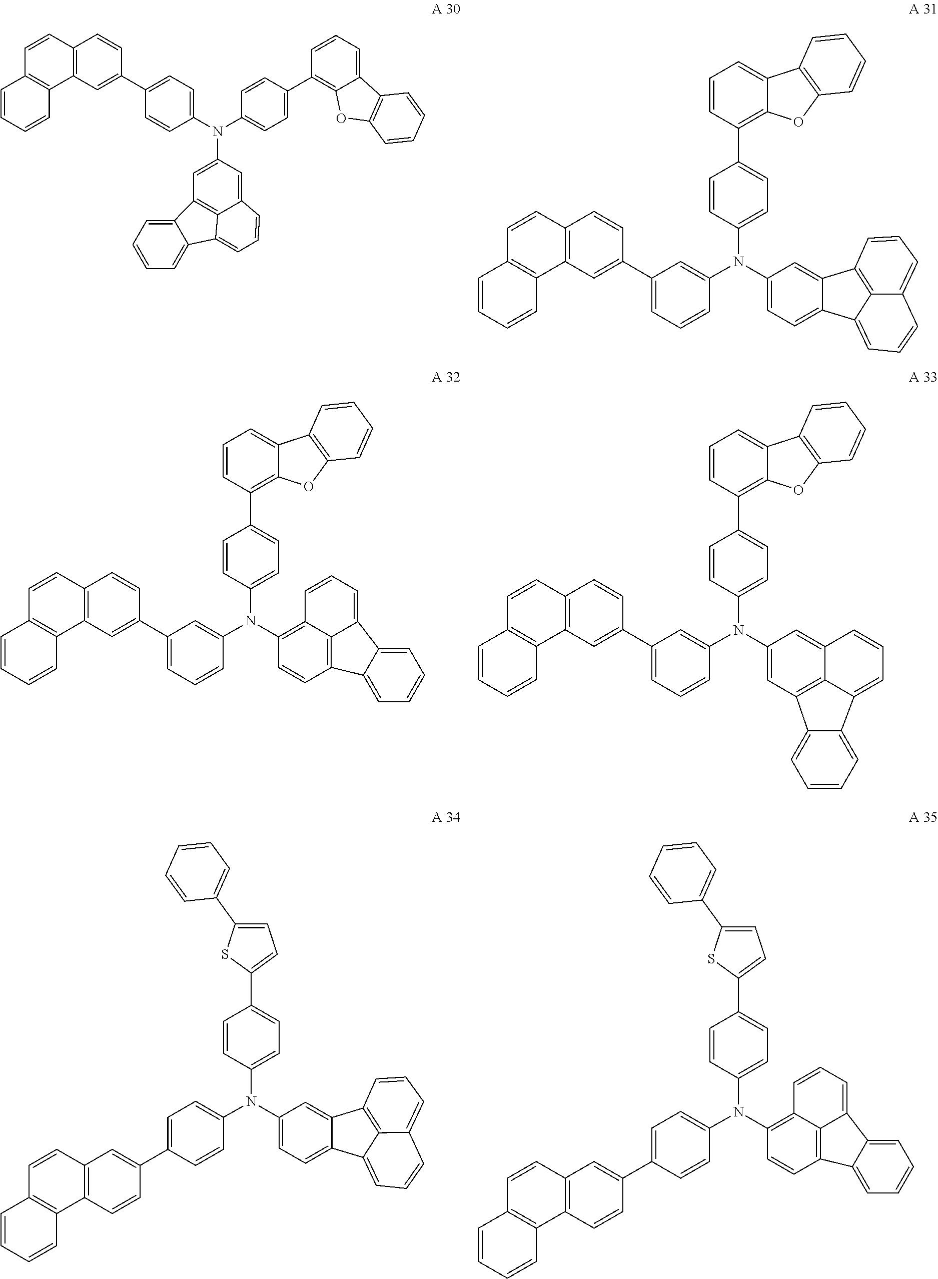

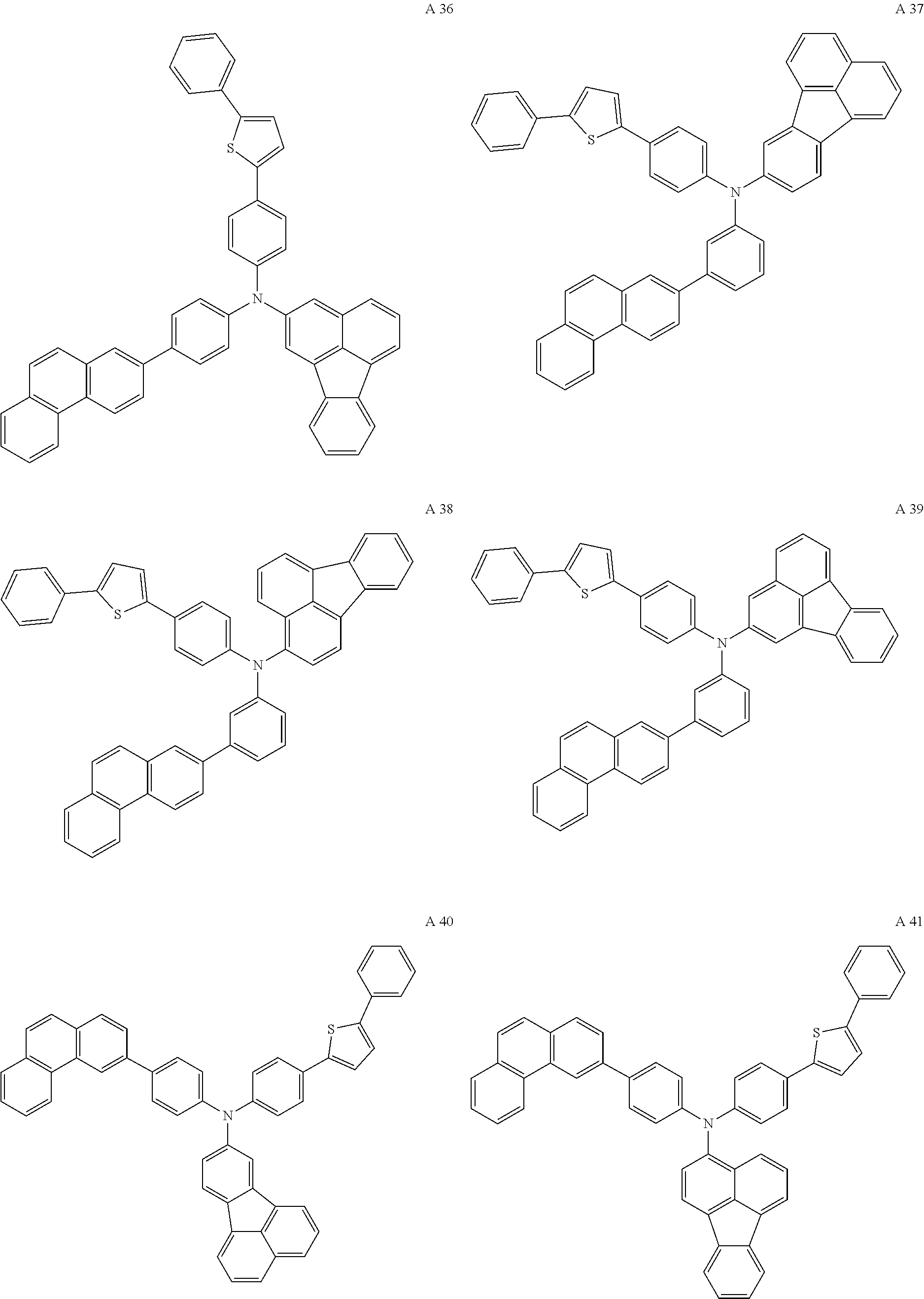

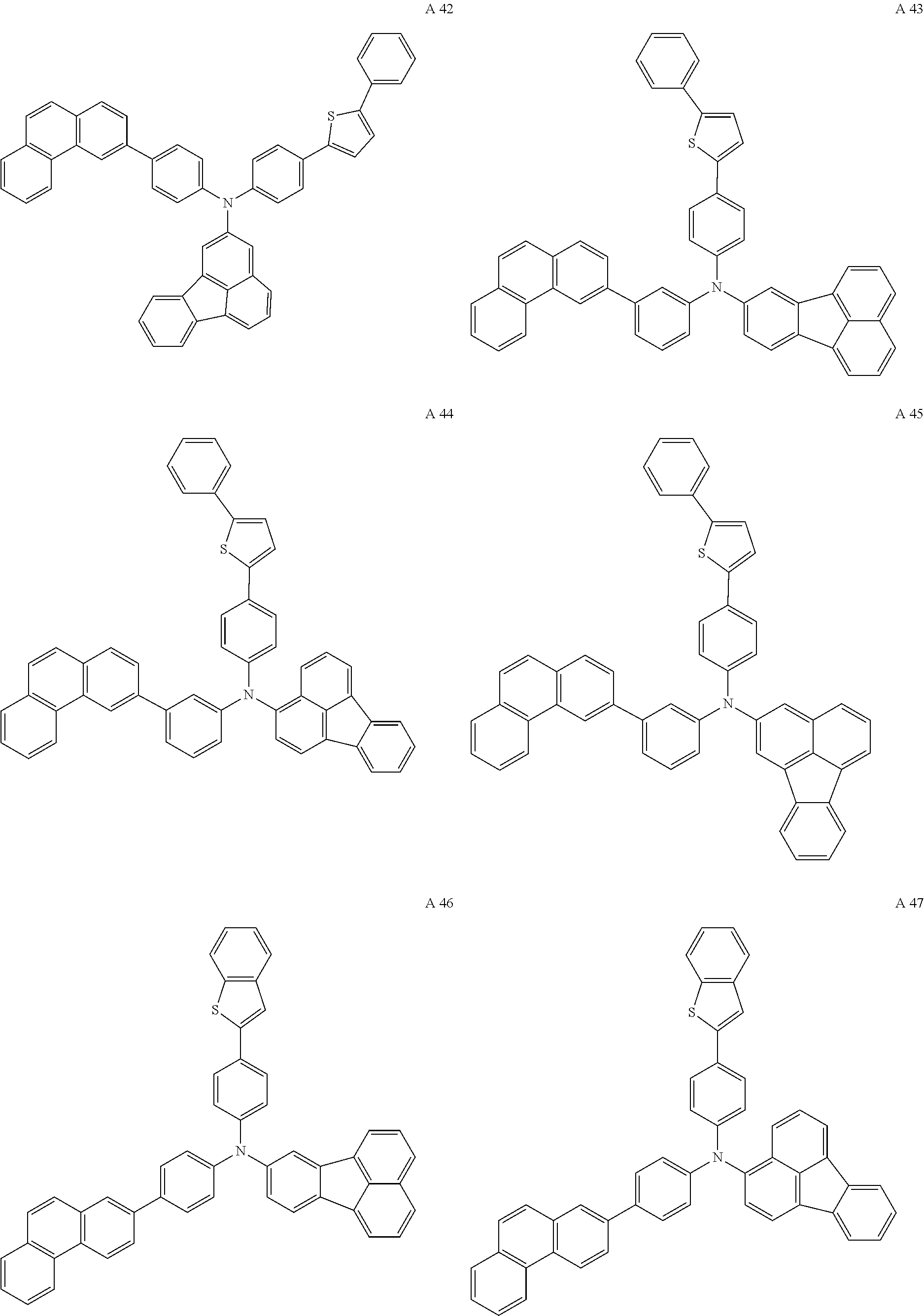

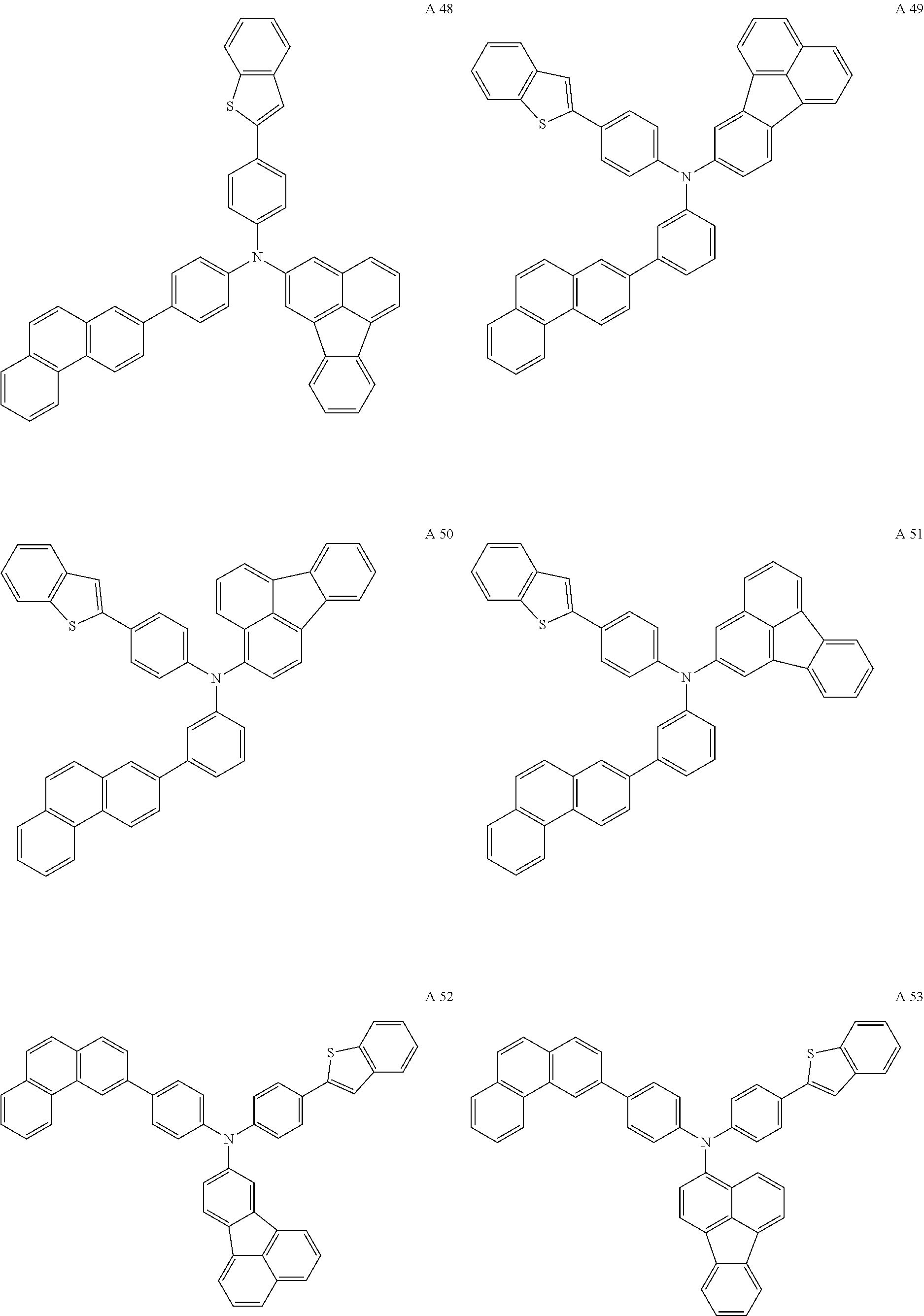

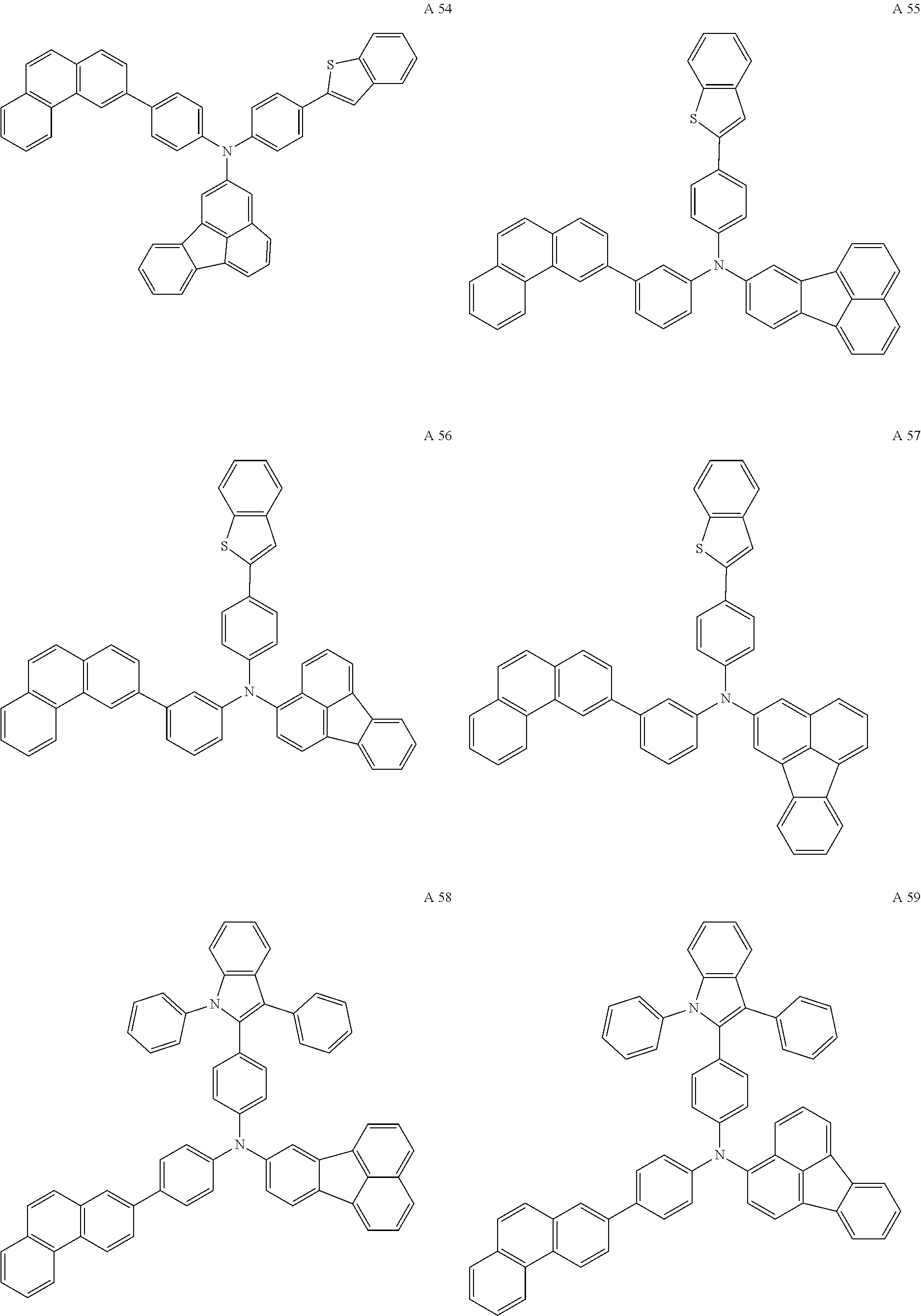

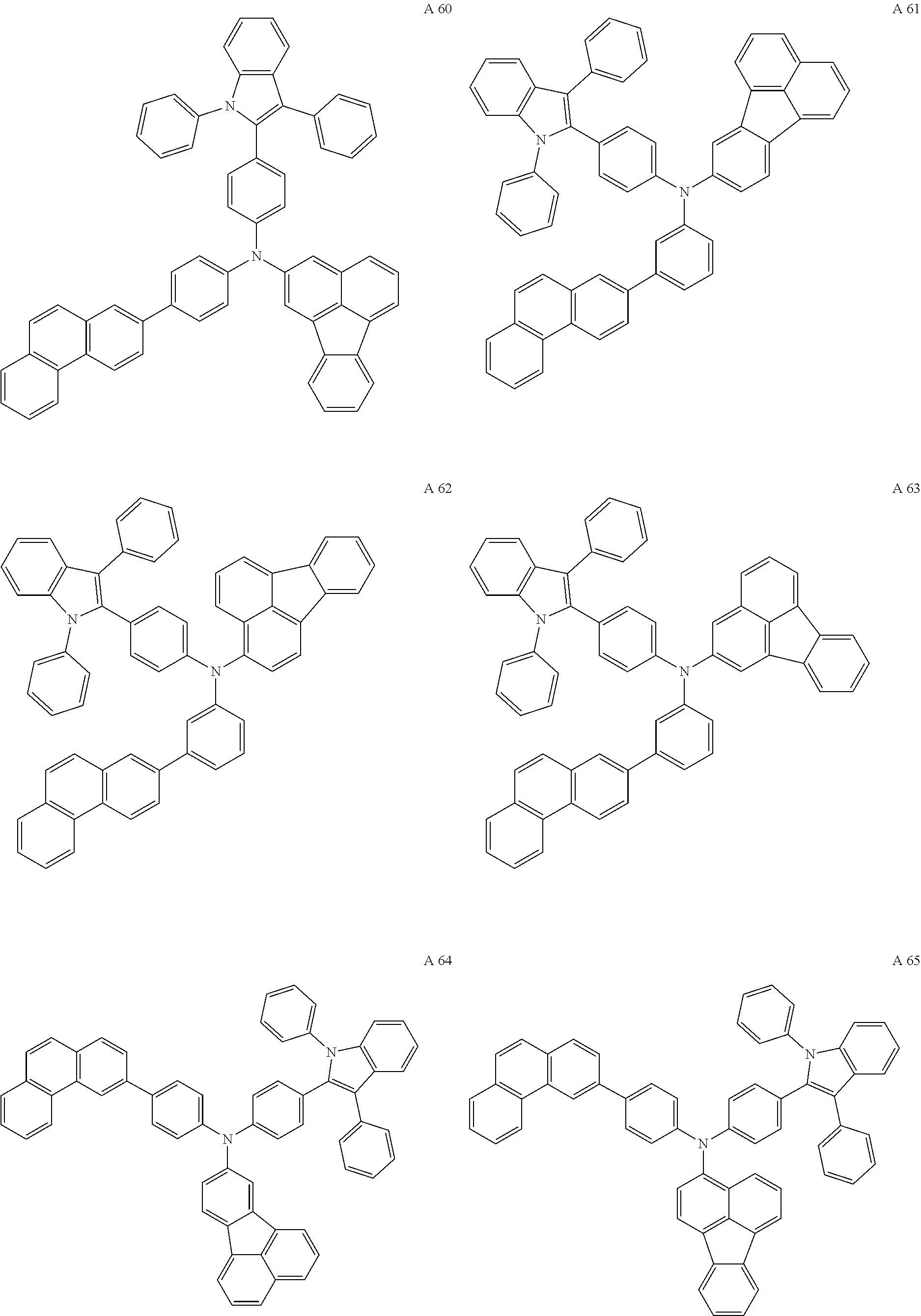

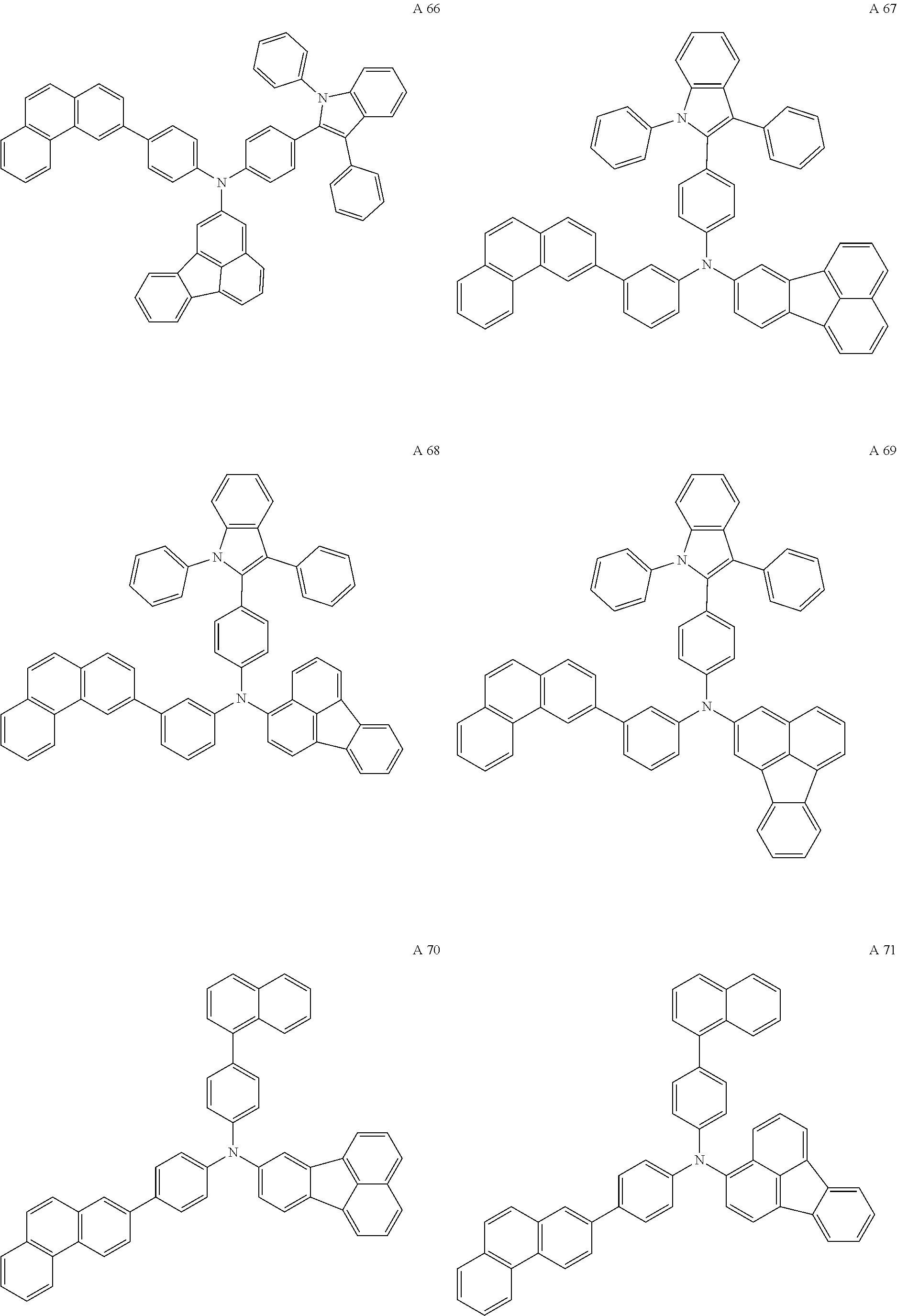

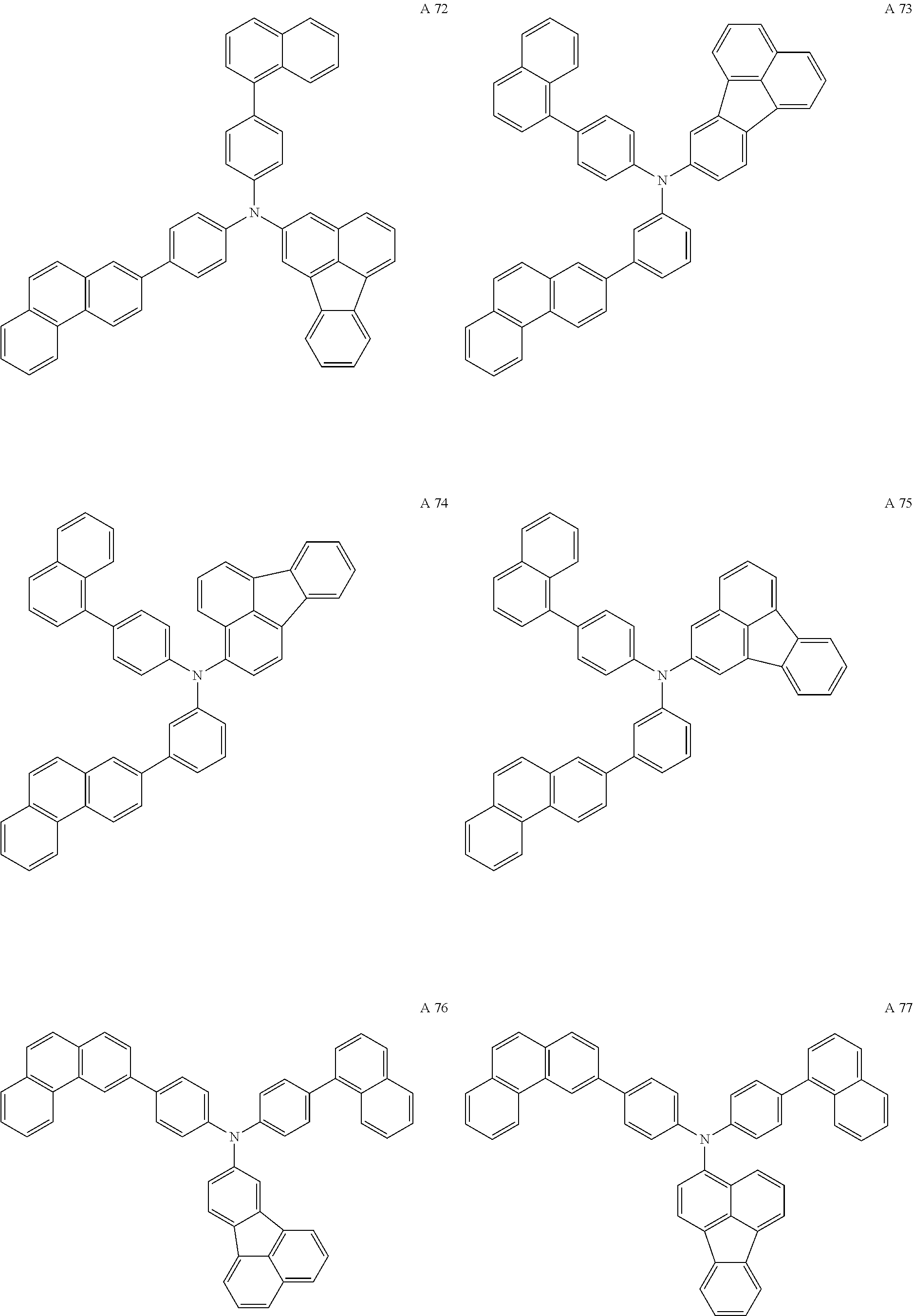

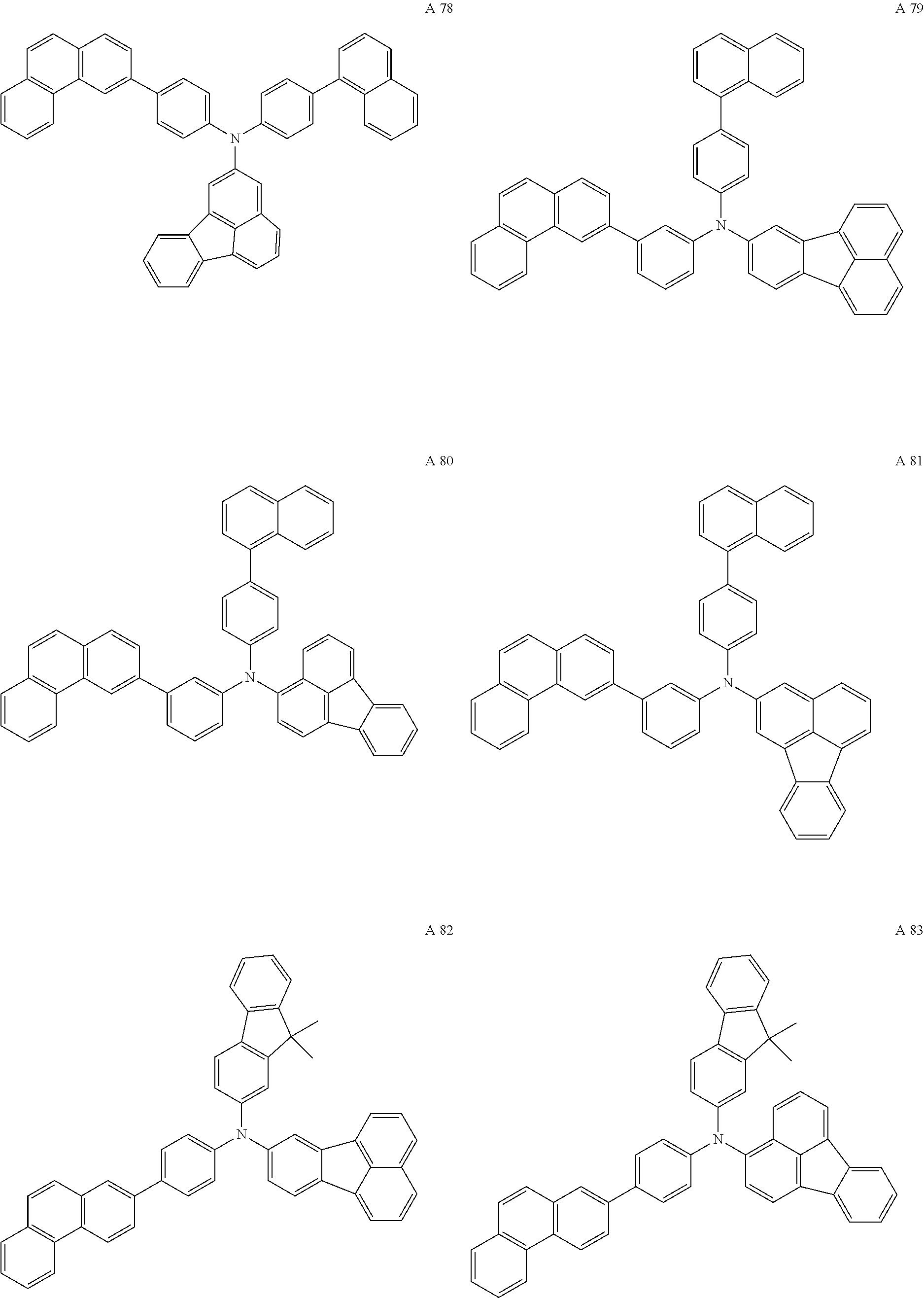

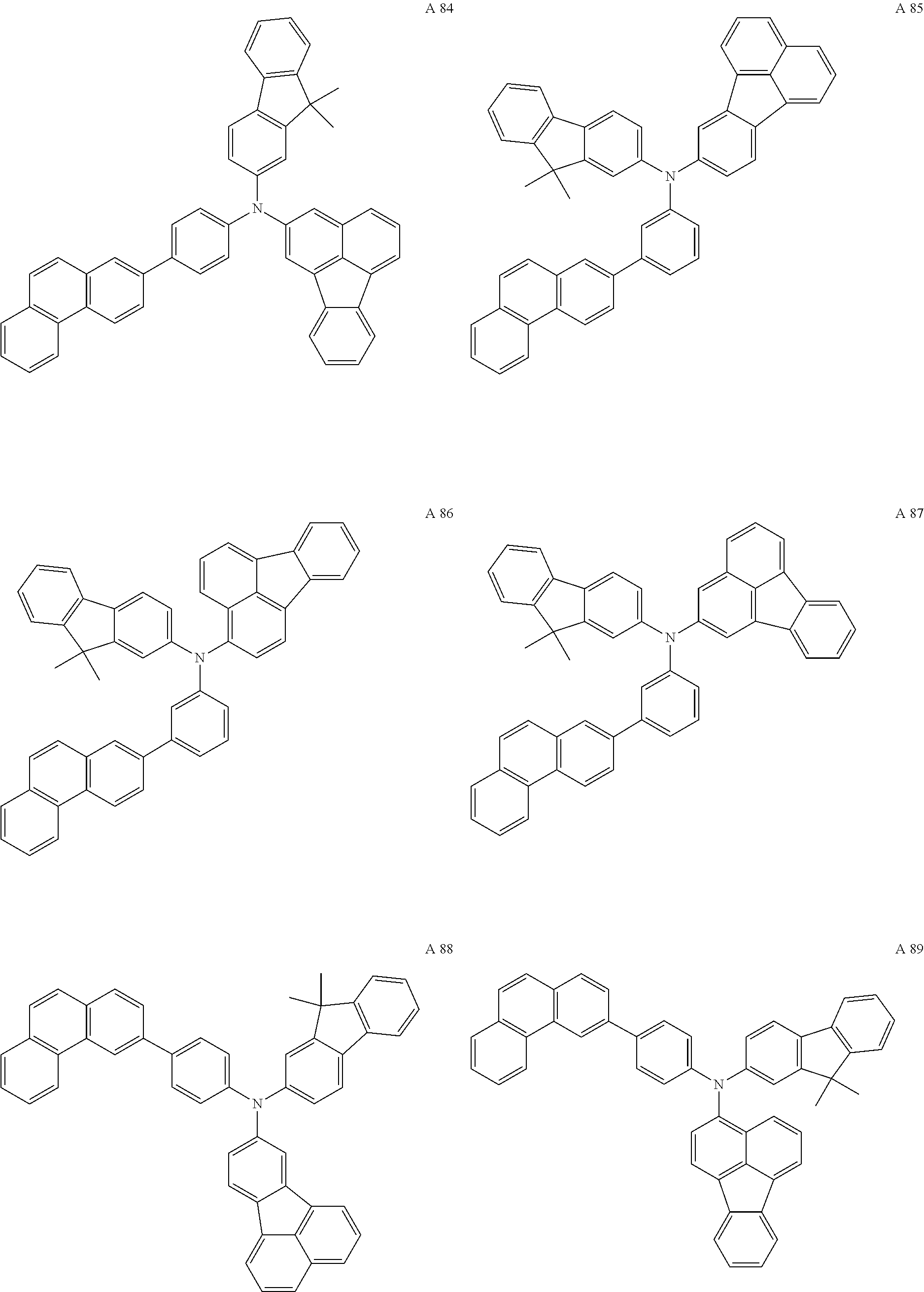

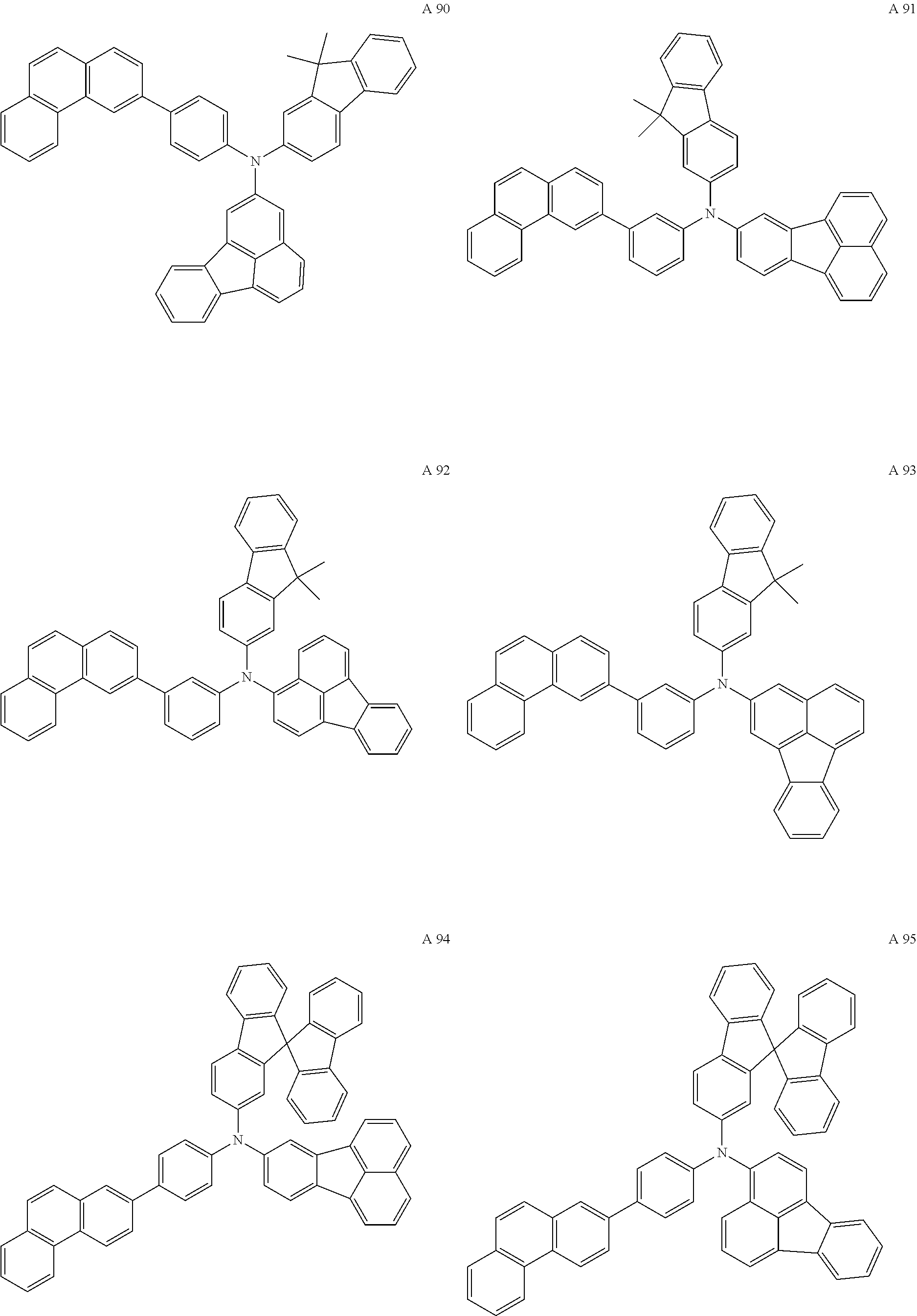

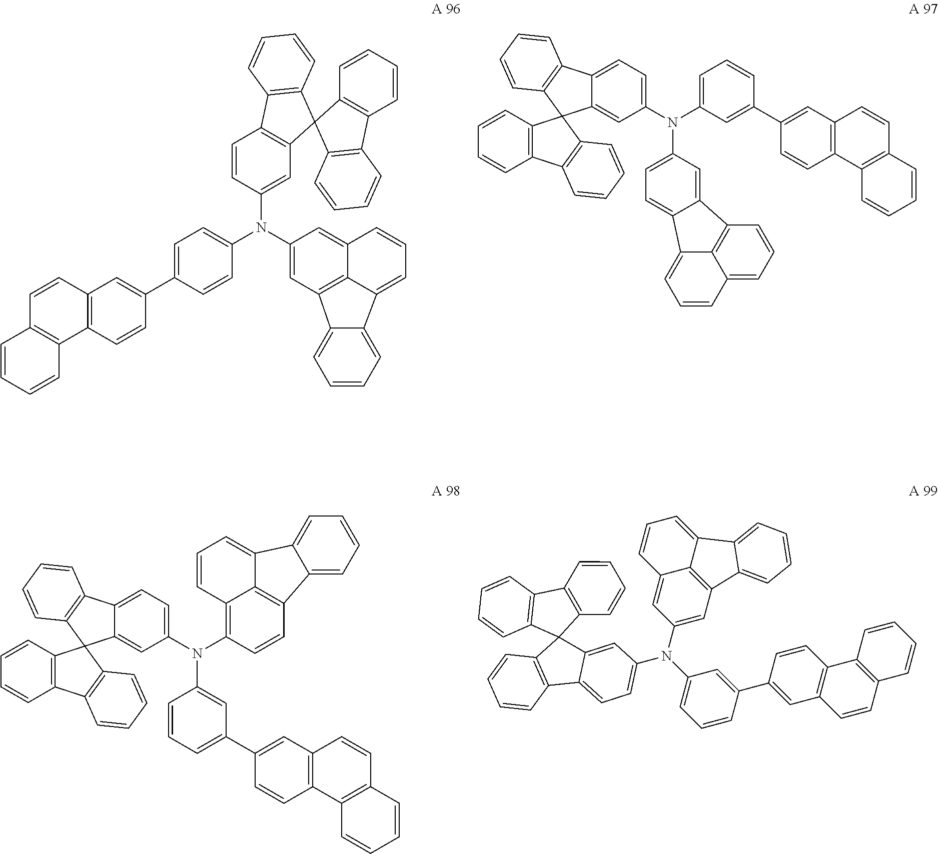 [SMILES: c1ccc2c(c1)-c1cccc3cc(N(c4ccc(-c5ccc6c(ccc7ccccc76)c5)cc4)c4ccc5c(c4)C4(c6ccccc6-c6ccccc64)c4ccccc4-5)cc-2c13 ; c1cc(-c2ccc3c(ccc4ccccc43)c2)cc(N(c2ccc3c(c2)-c2cccc4cccc-3c24)c2ccc3c(c2)C2(c4ccccc4-c4ccccc42)c2ccccc2-3)c1 ; c1cc(-c2ccc3c(ccc4ccccc43)c2)cc(N(c2ccc3c(c2)C2(c4ccccc4-c4ccccc42)c2ccccc2-3)c2ccc3c4c(cccc24)-c2ccccc2-3)c1 ; c1cc(-c2ccc3c(ccc4ccccc43)c2)cc(N(c2ccc3c(c2)C2(c4ccccc4-c4ccccc42)c2ccccc2-3)c2cc3c4c(cccc4c2)-c2ccccc2-3)c1]

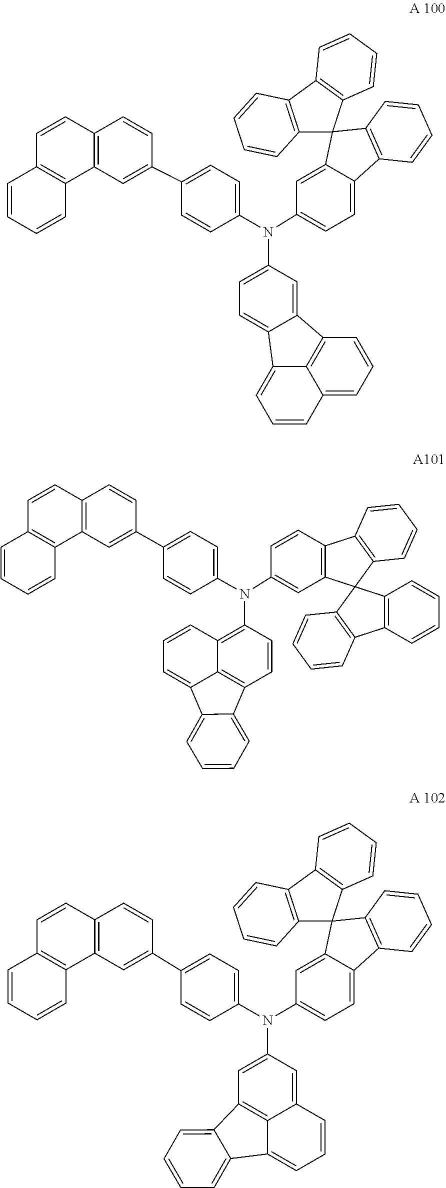 [SMILES: c1ccc2c(c1)-c1ccccc1C21c2ccccc2-c2ccc(N(c3ccc(-c4ccc5ccc6ccccc6c5c4)cc3)c3ccc4c(c3)-c3cccc5cccc-4c35)cc21 ; c1ccc2c(c1)-c1cccc3c(N(c4ccc(-c5ccc6ccc7ccccc7c6c5)cc4)c4ccc5c(c4)C4(c6ccccc6-c6ccccc64)c4ccccc4-5)ccc-2c13 ; c1ccc2c(c1)-c1cccc3cc(N(c4ccc(-c5ccc6ccc7ccccc7c6c5)cc4)c4ccc5c(c4)C4(c6ccccc6-c6ccccc64)c4ccccc4-5)cc-2c13]

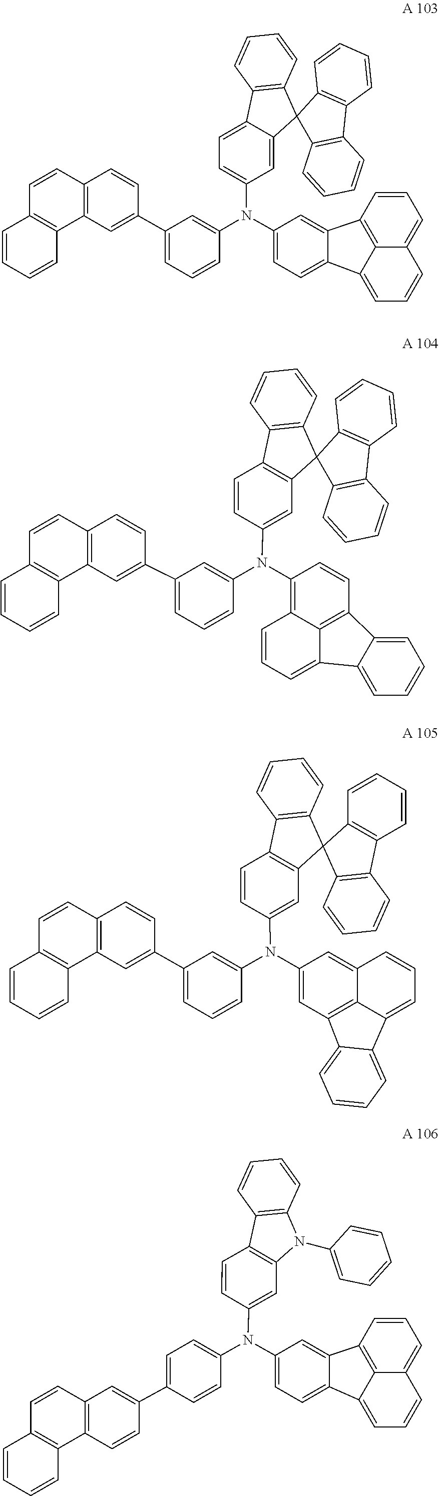 [SMILES: c1cc(-c2ccc3ccc4ccccc4c3c2)cc(N(c2ccc3c(c2)-c2cccc4cccc-3c24)c2ccc3c(c2)C2(c4ccccc4-c4ccccc42)c2ccccc2-3)c1 ; c1cc(-c2ccc3ccc4ccccc4c3c2)cc(N(c2ccc3c(c2)C2(c4ccccc4-c4ccccc42)c2ccccc2-3)c2ccc3c4c(cccc24)-c2ccccc2-3)c1 ; c1cc(-c2ccc3ccc4ccccc4c3c2)cc(N(c2ccc3c(c2)C2(c4ccccc4-c4ccccc42)c2ccccc2-3)c2cc3c4c(cccc4c2)-c2ccccc2-3)c1 ; c1ccc(-n2c3ccccc3c3ccc(N(c4ccc(-c5ccc6c(ccc7ccccc76)c5)cc4)c4ccc5c(c4)-c4cccc6cccc-5c46)cc32)cc1]

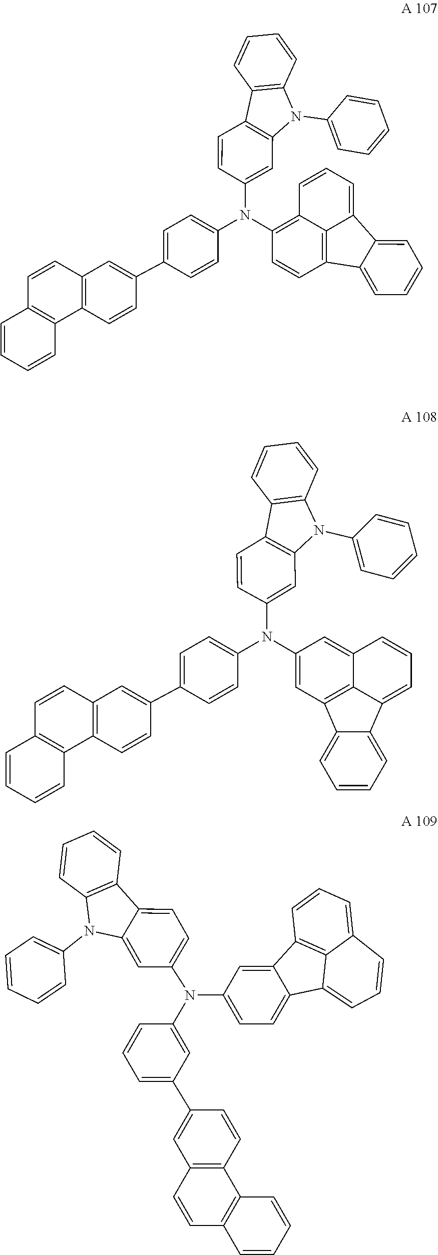 [SMILES: c1ccc(-n2c3ccccc3c3ccc(N(c4ccc(-c5ccc6c(ccc7ccccc76)c5)cc4)c4ccc5c6c(cccc46)-c4ccccc4-5)cc32)cc1 ; c1ccc(-n2c3ccccc3c3ccc(N(c4ccc(-c5ccc6c(ccc7ccccc76)c5)cc4)c4cc5c6c(cccc6c4)-c4ccccc4-5)cc32)cc1 ; c1ccc(-n2c3ccccc3c3ccc(N(c4cccc(-c5ccc6c(ccc7ccccc76)c5)c4)c4ccc5c(c4)-c4cccc6cccc-5c46)cc32)cc1]

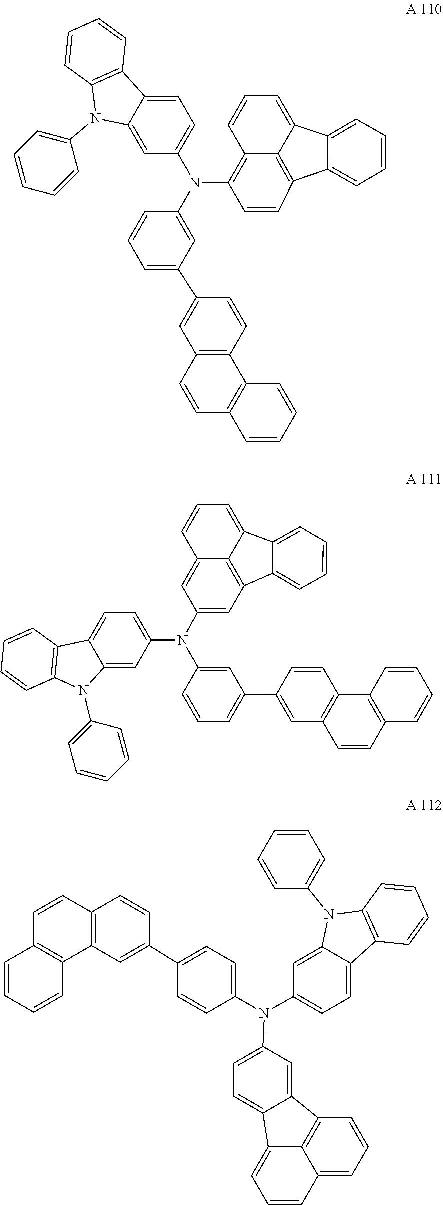 [SMILES: c1ccc(-n2c3ccccc3c3ccc(N(c4cccc(-c5ccc6c(ccc7ccccc76)c5)c4)c4ccc5c6c(cccc46)-c4ccccc4-5)cc32)cc1 ; c1ccc(-n2c3ccccc3c3ccc(N(c4cccc(-c5ccc6c(ccc7ccccc76)c5)c4)c4cc5c6c(cccc6c4)-c4ccccc4-5)cc32)cc1 ; c1ccc(-n2c3ccccc3c3ccc(N(c4ccc(-c5ccc6ccc7ccccc7c6c5)cc4)c4ccc5c(c4)-c4cccc6cccc-5c46)cc32)cc1]

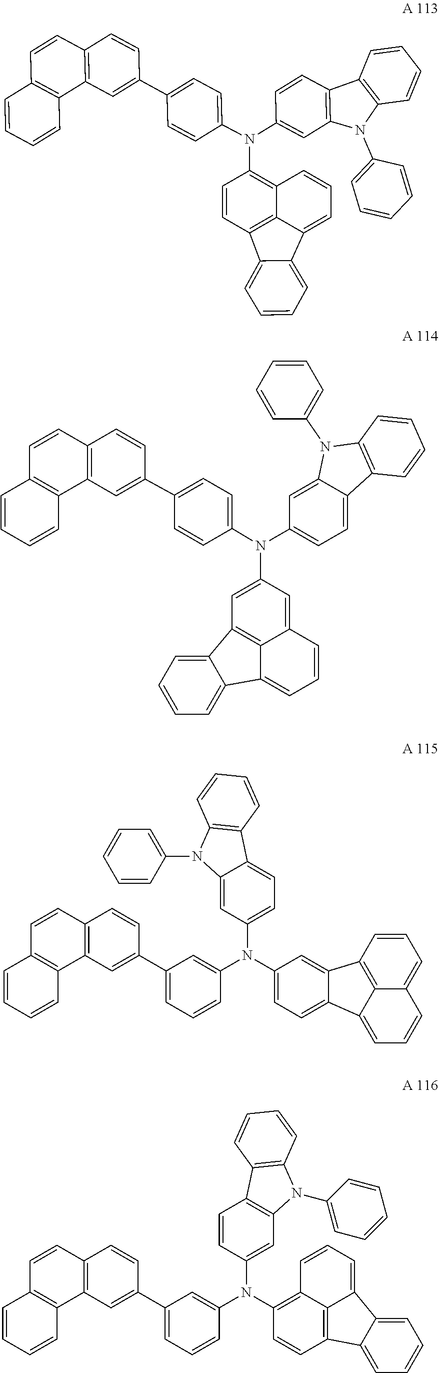 [SMILES: c1ccc(-n2c3ccccc3c3ccc(N(c4ccc(-c5ccc6ccc7ccccc7c6c5)cc4)c4ccc5c6c(cccc46)-c4ccccc4-5)cc32)cc1 ; c1ccc(-n2c3ccccc3c3ccc(N(c4ccc(-c5ccc6ccc7ccccc7c6c5)cc4)c4cc5c6c(cccc6c4)-c4ccccc4-5)cc32)cc1 ; c1ccc(-n2c3ccccc3c3ccc(N(c4cccc(-c5ccc6ccc7ccccc7c6c5)c4)c4ccc5c(c4)-c4cccc6cccc-5c46)cc32)cc1 ; c1ccc(-n2c3ccccc3c3ccc(N(c4cccc(-c5ccc6ccc7ccccc7c6c5)c4)c4ccc5c6c(cccc46)-c4ccccc4-5)cc32)cc1]

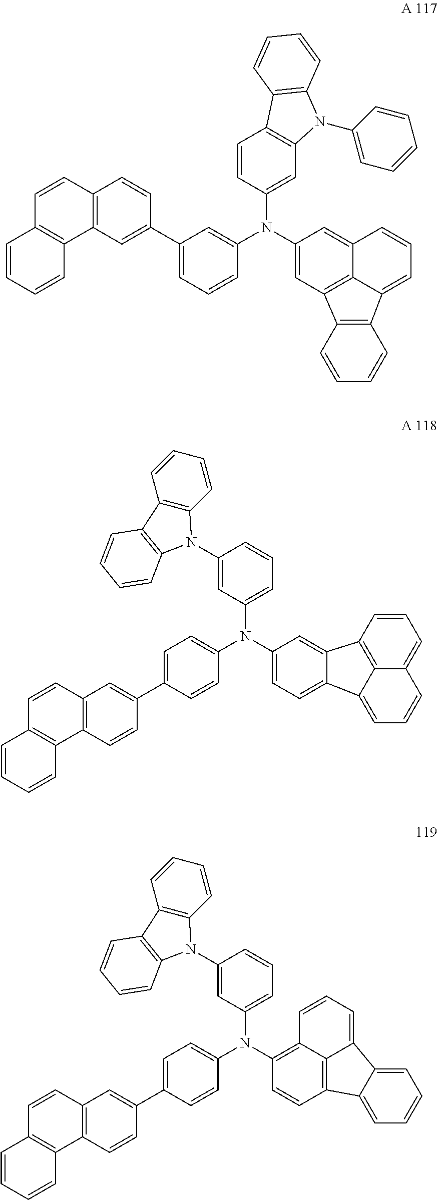 [SMILES: c1ccc(-n2c3ccccc3c3ccc(N(c4cccc(-c5ccc6ccc7ccccc7c6c5)c4)c4cc5c6c(cccc6c4)-c4ccccc4-5)cc32)cc1 ; c1cc(N(c2ccc(-c3ccc4c(ccc5ccccc54)c3)cc2)c2ccc3c(c2)-c2cccc4cccc-3c24)cc(-n2c3ccccc3c3ccccc32)c1 ; c1cc(N(c2ccc(-c3ccc4c(ccc5ccccc54)c3)cc2)c2ccc3c4c(cccc24)-c2ccccc2-3)cc(-n2c3ccccc3c3ccccc32)c1]

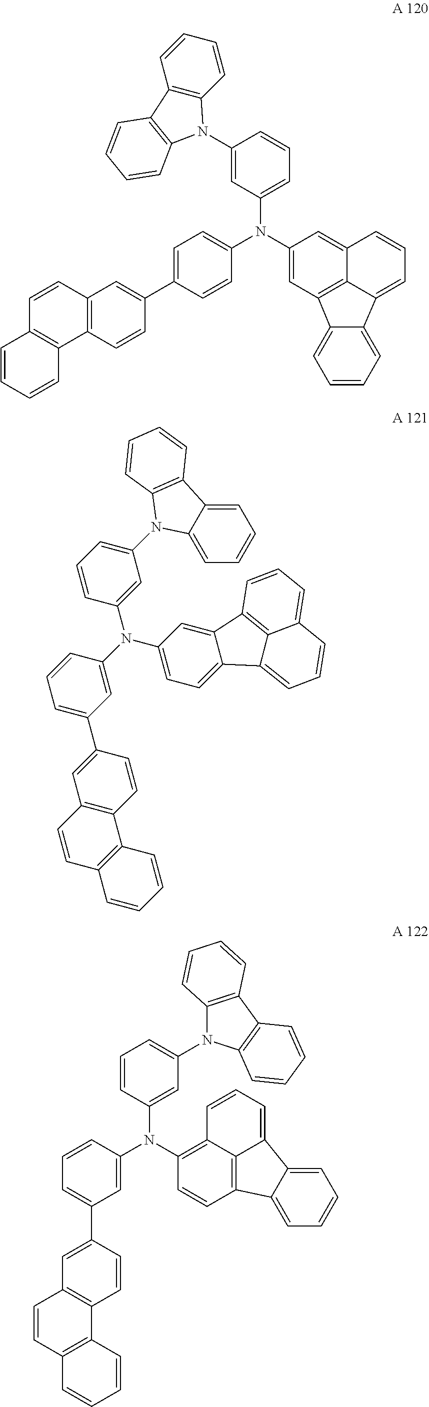 [SMILES: c1cc(N(c2ccc(-c3ccc4c(ccc5ccccc54)c3)cc2)c2cc3c4c(cccc4c2)-c2ccccc2-3)cc(-n2c3ccccc3c3ccccc32)c1 ; c1cc(-c2ccc3c(ccc4ccccc43)c2)cc(N(c2cccc(-n3c4ccccc4c4ccccc43)c2)c2ccc3c(c2)-c2cccc4cccc-3c24)c1 ; c1cc(-c2ccc3c(ccc4ccccc43)c2)cc(N(c2cccc(-n3c4ccccc4c4ccccc43)c2)c2ccc3c4c(cccc24)-c2ccccc2-3)c1]

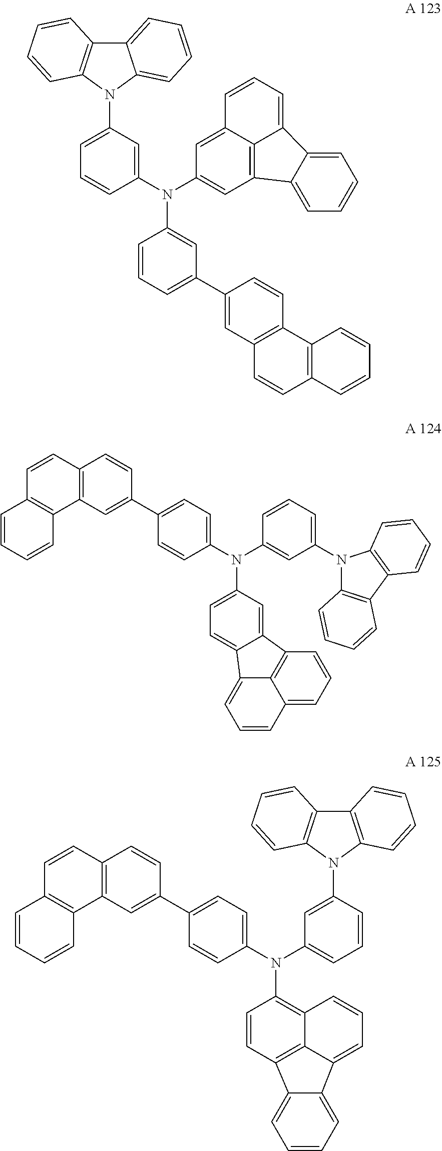 [SMILES: c1cc(-c2ccc3c(ccc4ccccc43)c2)cc(N(c2cccc(-n3c4ccccc4c4ccccc43)c2)c2cc3c4c(cccc4c2)-c2ccccc2-3)c1 ; c1cc(N(c2ccc(-c3ccc4ccc5ccccc5c4c3)cc2)c2ccc3c(c2)-c2cccc4cccc-3c24)cc(-n2c3ccccc3c3ccccc32)c1 ; c1cc(N(c2ccc(-c3ccc4ccc5ccccc5c4c3)cc2)c2ccc3c4c(cccc24)-c2ccccc2-3)cc(-n2c3ccccc3c3ccccc32)c1]

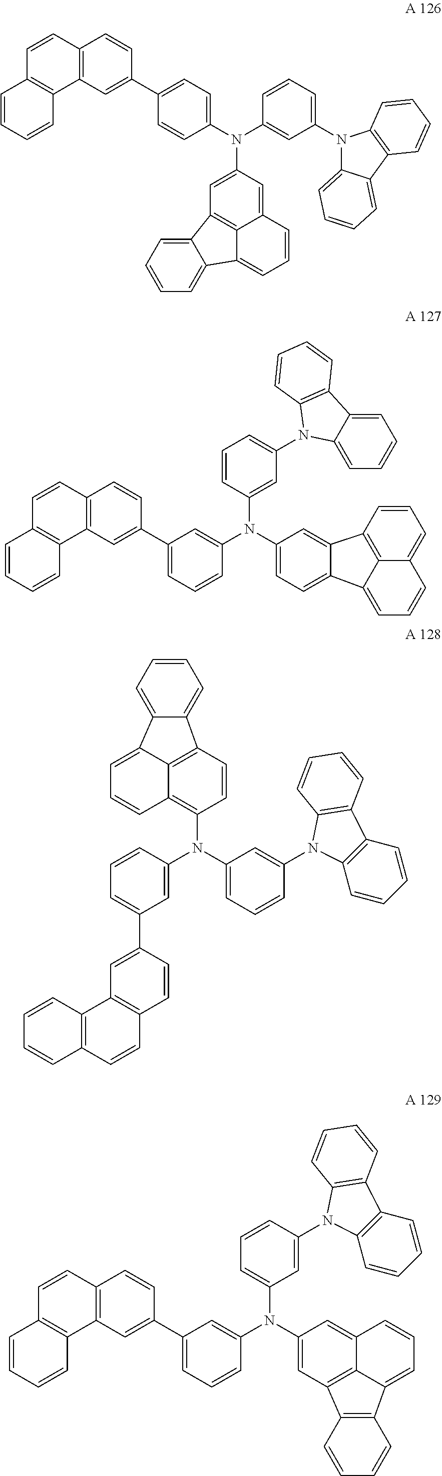 [SMILES: c1cc(N(c2ccc(-c3ccc4ccc5ccccc5c4c3)cc2)c2cc3c4c(cccc4c2)-c2ccccc2-3)cc(-n2c3ccccc3c3ccccc32)c1 ; c1cc(-c2ccc3ccc4ccccc4c3c2)cc(N(c2cccc(-n3c4ccccc4c4ccccc43)c2)c2ccc3c(c2)-c2cccc4cccc-3c24)c1 ; c1cc(-c2ccc3ccc4ccccc4c3c2)cc(N(c2cccc(-n3c4ccccc4c4ccccc43)c2)c2ccc3c4c(cccc24)-c2ccccc2-3)c1 ; c1cc(-c2ccc3ccc4ccccc4c3c2)cc(N(c2cccc(-n3c4ccccc4c4ccccc43)c2)c2cc3c4c(cccc4c2)-c2ccccc2-3)c1]

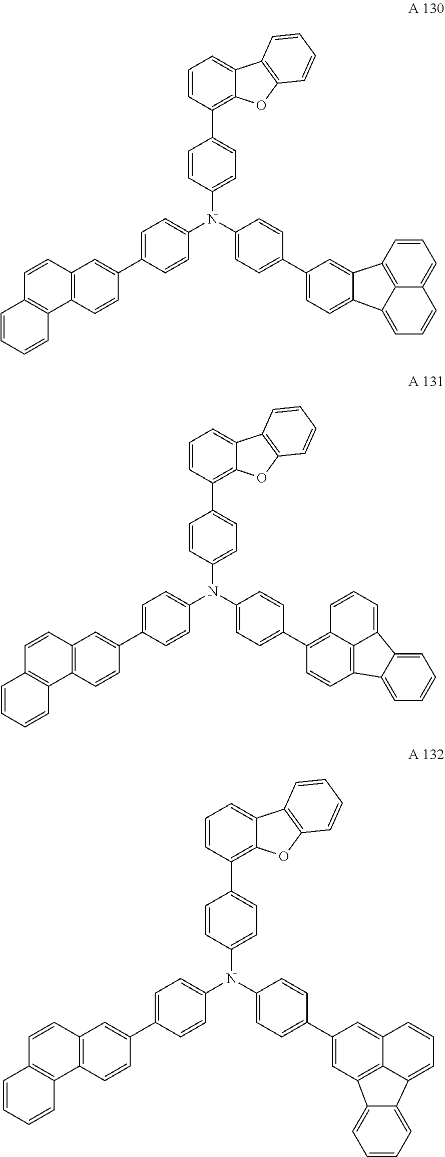 [SMILES: c1cc2c3c(cccc3c1)-c1cc(-c3ccc(N(c4ccc(-c5ccc6c(ccc7ccccc76)c5)cc4)c4ccc(-c5cccc6c5oc5ccccc56)cc4)cc3)ccc1-2 ; c1ccc2c(c1)-c1cccc3c(-c4ccc(N(c5ccc(-c6ccc7c(ccc8ccccc87)c6)cc5)c5ccc(-c6cccc7c6oc6ccccc67)cc5)cc4)ccc-2c13 ; c1ccc2c(c1)-c1cccc3cc(-c4ccc(N(c5ccc(-c6ccc7c(ccc8ccccc87)c6)cc5)c5ccc(-c6cccc7c6oc6ccccc67)cc5)cc4)cc-2c13]

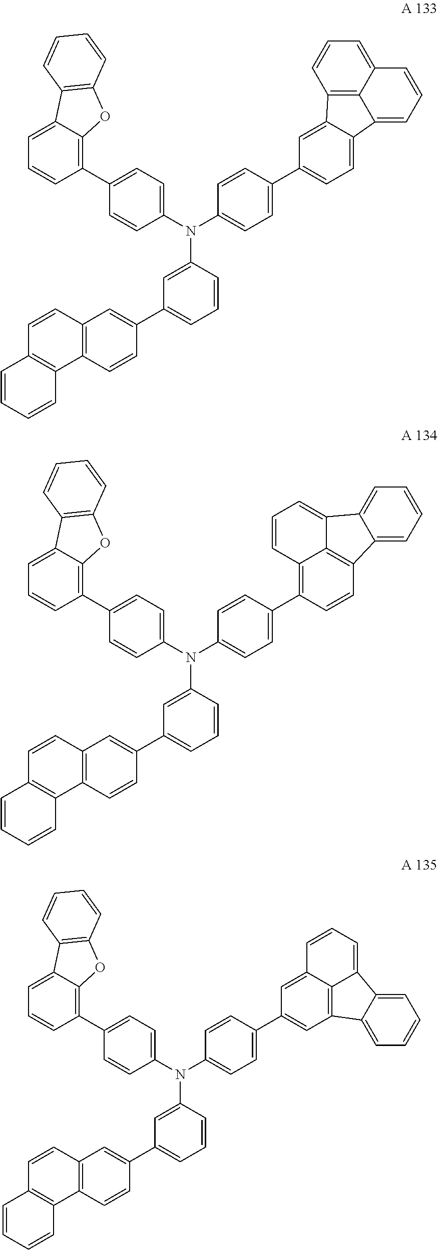 [SMILES: c1cc(-c2ccc3c(ccc4ccccc43)c2)cc(N(c2ccc(-c3ccc4c(c3)-c3cccc5cccc-4c35)cc2)c2ccc(-c3cccc4c3oc3ccccc34)cc2)c1 ; c1cc(-c2ccc3c(ccc4ccccc43)c2)cc(N(c2ccc(-c3ccc4c5c(cccc35)-c3ccccc3-4)cc2)c2ccc(-c3cccc4c3oc3ccccc34)cc2)c1 ; c1cc(-c2ccc3c(ccc4ccccc43)c2)cc(N(c2ccc(-c3cc4c5c(cccc5c3)-c3ccccc3-4)cc2)c2ccc(-c3cccc4c3oc3ccccc34)cc2)c1]

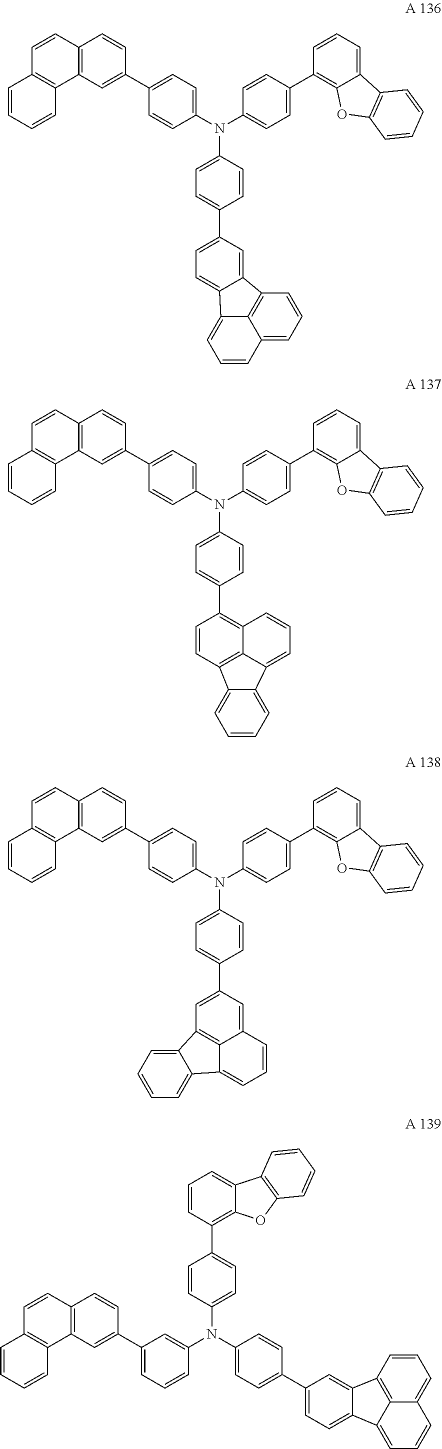 [SMILES: c1cc2c3c(cccc3c1)-c1cc(-c3ccc(N(c4ccc(-c5ccc6ccc7ccccc7c6c5)cc4)c4ccc(-c5cccc6c5oc5ccccc56)cc4)cc3)ccc1-2 ; c1ccc2c(c1)-c1cccc3c(-c4ccc(N(c5ccc(-c6ccc7ccc8ccccc8c7c6)cc5)c5ccc(-c6cccc7c6oc6ccccc67)cc5)cc4)ccc-2c13 ; c1ccc2c(c1)-c1cccc3cc(-c4ccc(N(c5ccc(-c6ccc7ccc8ccccc8c7c6)cc5)c5ccc(-c6cccc7c6oc6ccccc67)cc5)cc4)cc-2c13 ; c1cc(-c2ccc3ccc4ccccc4c3c2)cc(N(c2ccc(-c3ccc4c(c3)-c3cccc5cccc-4c35)cc2)c2ccc(-c3cccc4c3oc3ccccc34)cc2)c1]

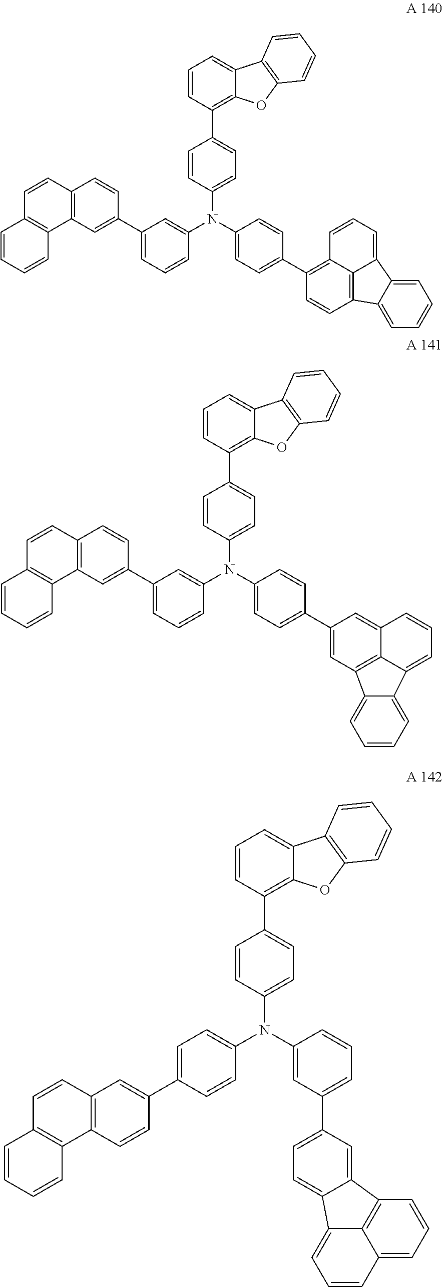 [SMILES: c1cc(-c2ccc3ccc4ccccc4c3c2)cc(N(c2ccc(-c3ccc4c5c(cccc35)-c3ccccc3-4)cc2)c2ccc(-c3cccc4c3oc3ccccc34)cc2)c1 ; c1cc(-c2ccc3ccc4ccccc4c3c2)cc(N(c2ccc(-c3cc4c5c(cccc5c3)-c3ccccc3-4)cc2)c2ccc(-c3cccc4c3oc3ccccc34)cc2)c1 ; c1cc(-c2ccc3c(c2)-c2cccc4cccc-3c24)cc(N(c2ccc(-c3ccc4c(ccc5ccccc54)c3)cc2)c2ccc(-c3cccc4c3oc3ccccc34)cc2)c1]

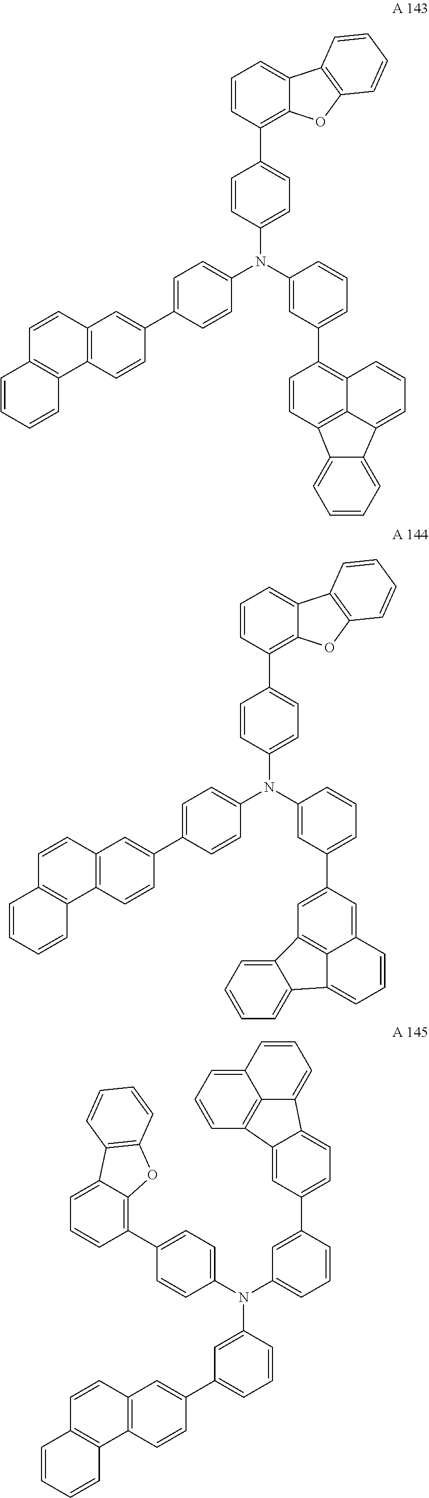

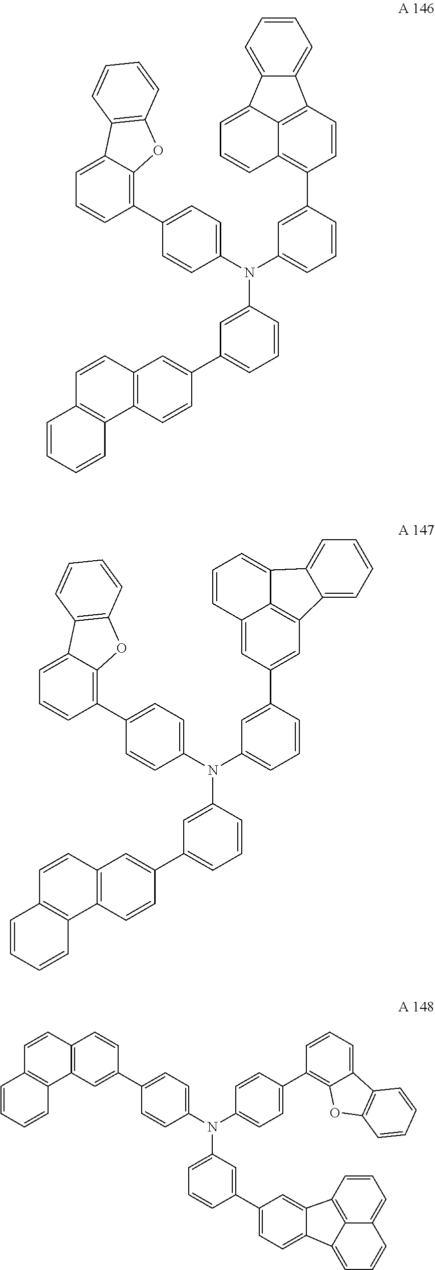 [SMILES: c1cc(-c2ccc3c(ccc4ccccc43)c2)cc(N(c2ccc(-c3cccc4c3oc3ccccc34)cc2)c2cccc(-c3ccc4c5c(cccc35)-c3ccccc3-4)c2)c1 ; c1cc(-c2cc3c4c(cccc4c2)-c2ccccc2-3)cc(N(c2ccc(-c3cccc4c3oc3ccccc34)cc2)c2cccc(-c3ccc4c(ccc5ccccc54)c3)c2)c1 ; c1cc(-c2ccc3c(c2)-c2cccc4cccc-3c24)cc(N(c2ccc(-c3ccc4ccc5ccccc5c4c3)cc2)c2ccc(-c3cccc4c3oc3ccccc34)cc2)c1]

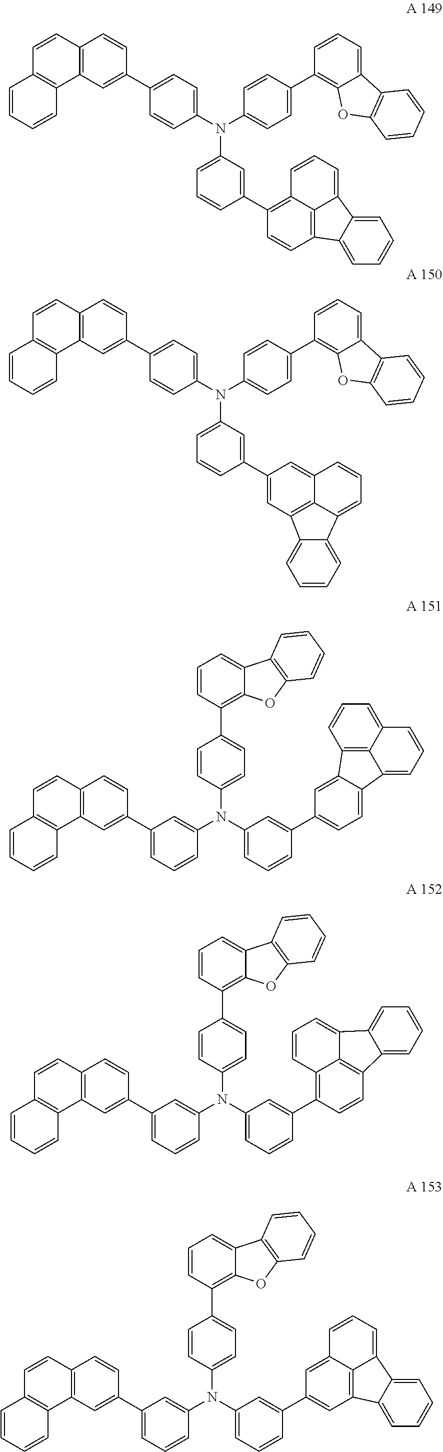

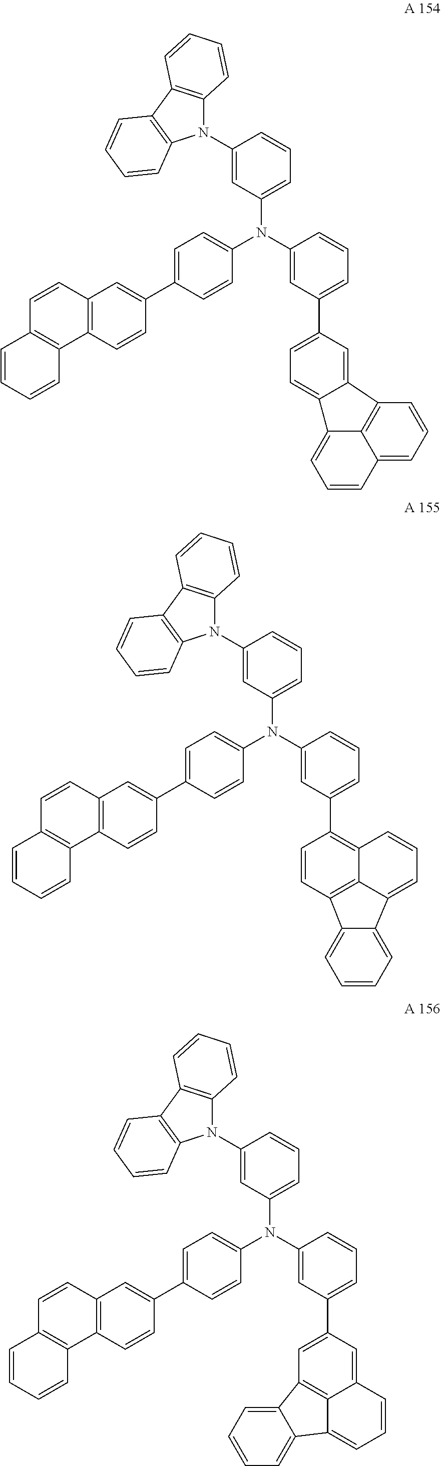 [SMILES: c1cc(-c2ccc3c(c2)-c2cccc4cccc-3c24)cc(N(c2ccc(-c3ccc4c(ccc5ccccc54)c3)cc2)c2cccc(-n3c4ccccc4c4ccccc43)c2)c1 ; c1cc(-c2ccc3c4c(cccc24)-c2ccccc2-3)cc(N(c2ccc(-c3ccc4c(ccc5ccccc54)c3)cc2)c2cccc(-n3c4ccccc4c4ccccc43)c2)c1 ; c1cc(-c2cc3c4c(cccc4c2)-c2ccccc2-3)cc(N(c2ccc(-c3ccc4c(ccc5ccccc54)c3)cc2)c2cccc(-n3c4ccccc4c4ccccc43)c2)c1]

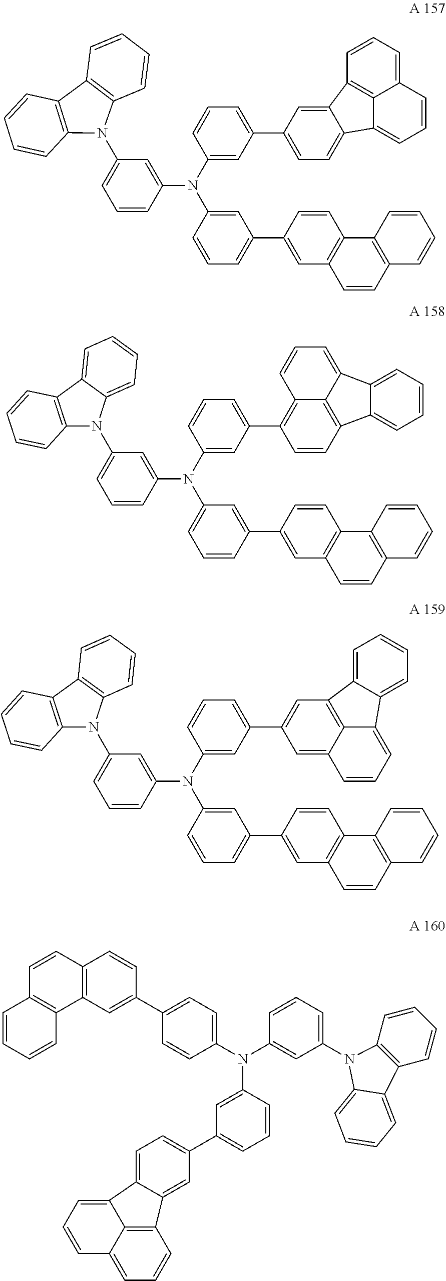

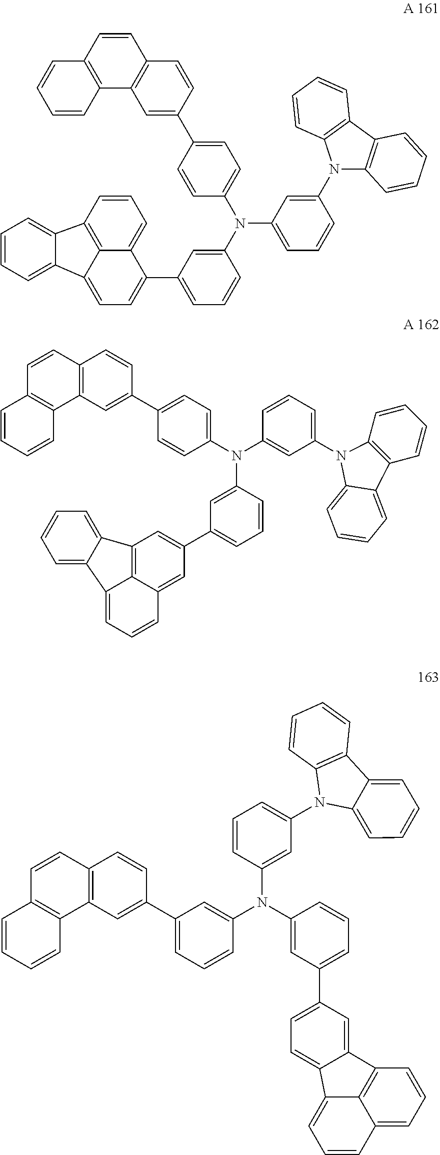 [SMILES: c1cc(-c2ccc3c4c(cccc24)-c2ccccc2-3)cc(N(c2ccc(-c3ccc4ccc5ccccc5c4c3)cc2)c2cccc(-n3c4ccccc4c4ccccc43)c2)c1 ; c1cc(-c2cc3c4c(cccc4c2)-c2ccccc2-3)cc(N(c2ccc(-c3ccc4ccc5ccccc5c4c3)cc2)c2cccc(-n3c4ccccc4c4ccccc43)c2)c1 ; c1cc(-c2ccc3c(c2)-c2cccc4cccc-3c24)cc(N(c2cccc(-c3ccc4ccc5ccccc5c4c3)c2)c2cccc(-n3c4ccccc4c4ccccc43)c2)c1]

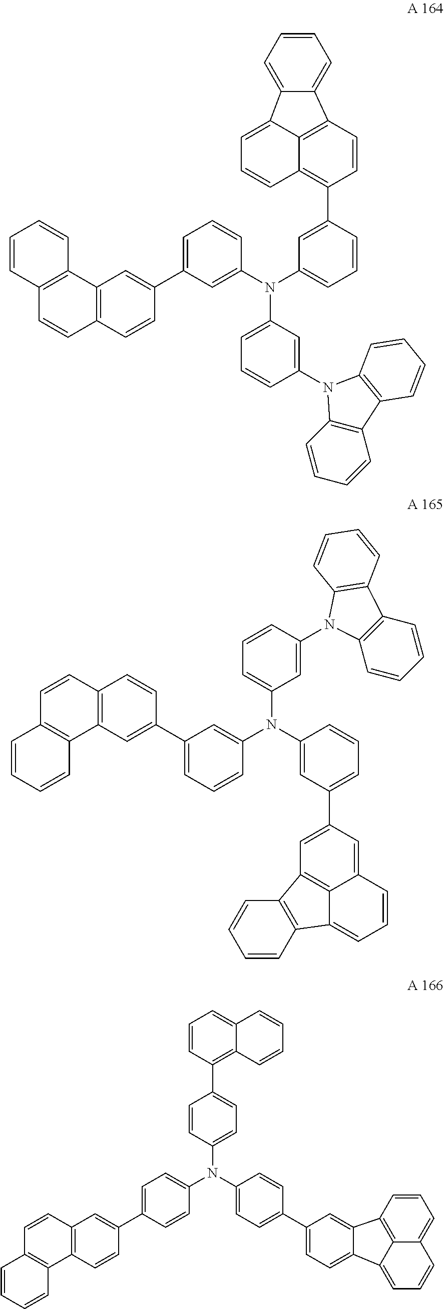

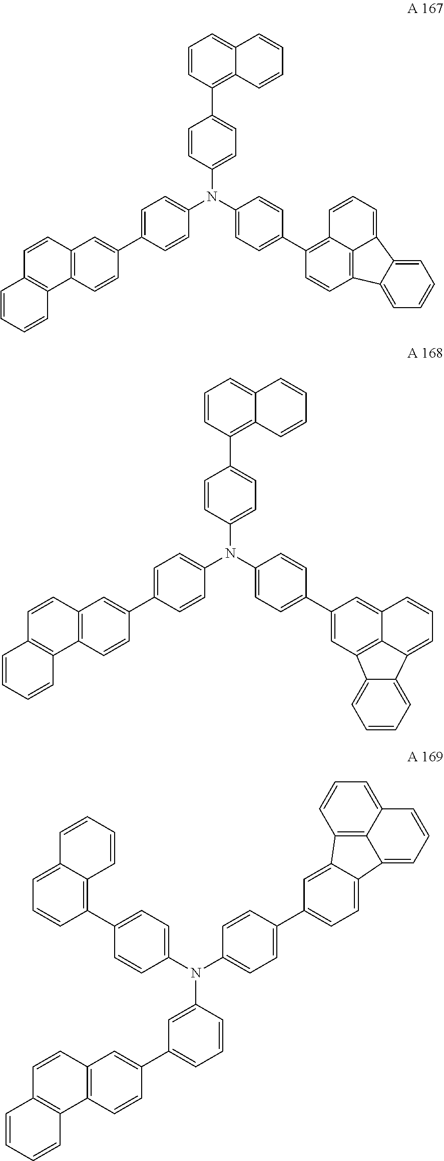 [SMILES: c1ccc2c(c1)-c1cccc3c(-c4ccc(N(c5ccc(-c6ccc7c(ccc8ccccc87)c6)cc5)c5ccc(-c6cccc7ccccc67)cc5)cc4)ccc-2c13 ; c1ccc2c(c1)-c1cccc3cc(-c4ccc(N(c5ccc(-c6ccc7c(ccc8ccccc87)c6)cc5)c5ccc(-c6cccc7ccccc67)cc5)cc4)cc-2c13 ; c1cc(-c2ccc3c(ccc4ccccc43)c2)cc(N(c2ccc(-c3ccc4c(c3)-c3cccc5cccc-4c35)cc2)c2ccc(-c3cccc4ccccc34)cc2)c1]

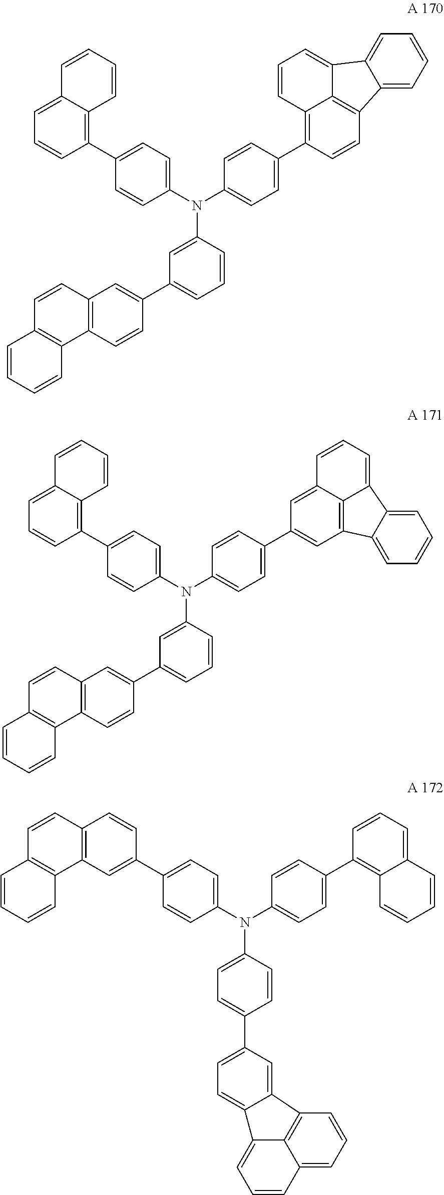 [SMILES: c1cc(-c2ccc3c(ccc4ccccc43)c2)cc(N(c2ccc(-c3cccc4ccccc34)cc2)c2ccc(-c3ccc4c5c(cccc35)-c3ccccc3-4)cc2)c1 ; c1cc(-c2ccc3c(ccc4ccccc43)c2)cc(N(c2ccc(-c3cc4c5c(cccc5c3)-c3ccccc3-4)cc2)c2ccc(-c3cccc4ccccc34)cc2)c1 ; c1ccc2c(-c3ccc(N(c4ccc(-c5ccc6c(c5)-c5cccc7cccc-6c57)cc4)c4ccc(-c5ccc6ccc7ccccc7c6c5)cc4)cc3)cccc2c1]

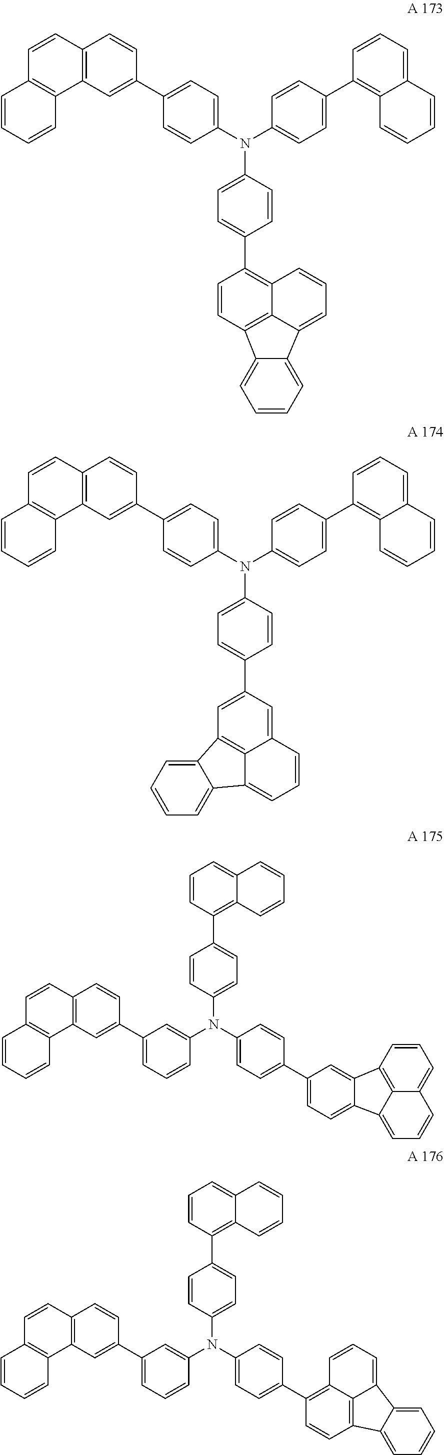 [SMILES: c1ccc2c(c1)-c1cccc3c(-c4ccc(N(c5ccc(-c6ccc7ccc8ccccc8c7c6)cc5)c5ccc(-c6cccc7ccccc67)cc5)cc4)ccc-2c13 ; c1ccc2c(c1)-c1cccc3cc(-c4ccc(N(c5ccc(-c6ccc7ccc8ccccc8c7c6)cc5)c5ccc(-c6cccc7ccccc67)cc5)cc4)cc-2c13 ; c1cc(-c2ccc3ccc4ccccc4c3c2)cc(N(c2ccc(-c3ccc4c(c3)-c3cccc5cccc-4c35)cc2)c2ccc(-c3cccc4ccccc34)cc2)c1 ; c1cc(-c2ccc3ccc4ccccc4c3c2)cc(N(c2ccc(-c3cccc4ccccc34)cc2)c2ccc(-c3ccc4c5c(cccc35)-c3ccccc3-4)cc2)c1]

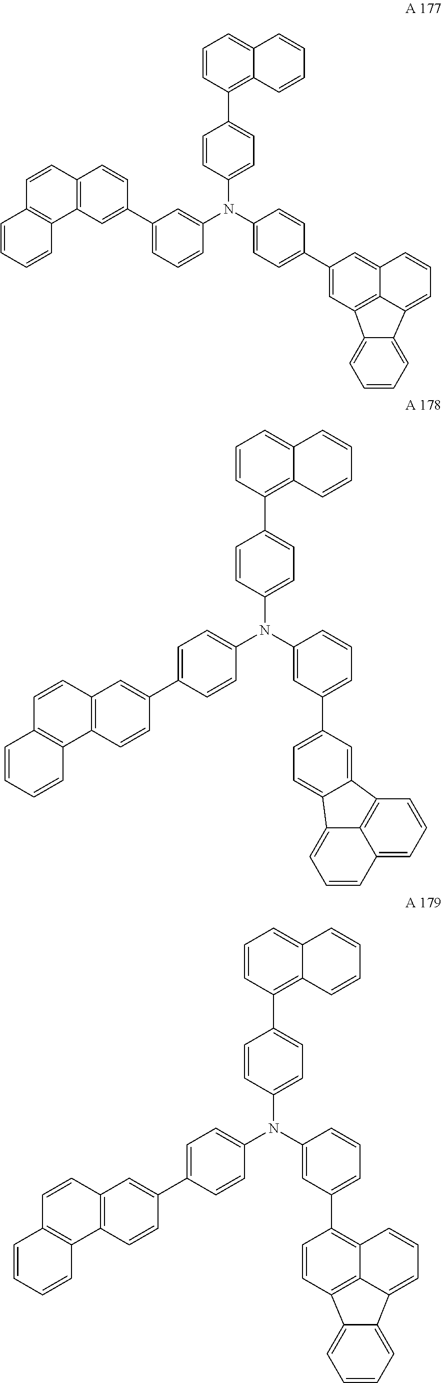

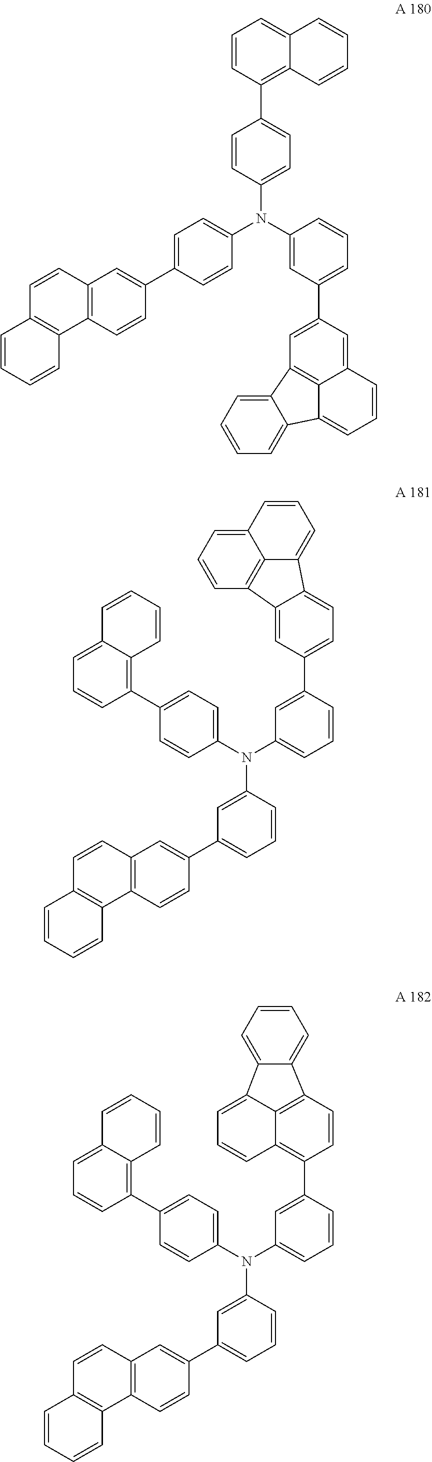 [SMILES: c1cc(-c2cc3c4c(cccc4c2)-c2ccccc2-3)cc(N(c2ccc(-c3ccc4c(ccc5ccccc54)c3)cc2)c2ccc(-c3cccc4ccccc34)cc2)c1 ; c1cc(-c2ccc3c(c2)-c2cccc4cccc-3c24)cc(N(c2ccc(-c3cccc4ccccc34)cc2)c2cccc(-c3ccc4c(ccc5ccccc54)c3)c2)c1 ; c1cc(-c2ccc3c(ccc4ccccc43)c2)cc(N(c2ccc(-c3cccc4ccccc34)cc2)c2cccc(-c3ccc4c5c(cccc35)-c3ccccc3-4)c2)c1]

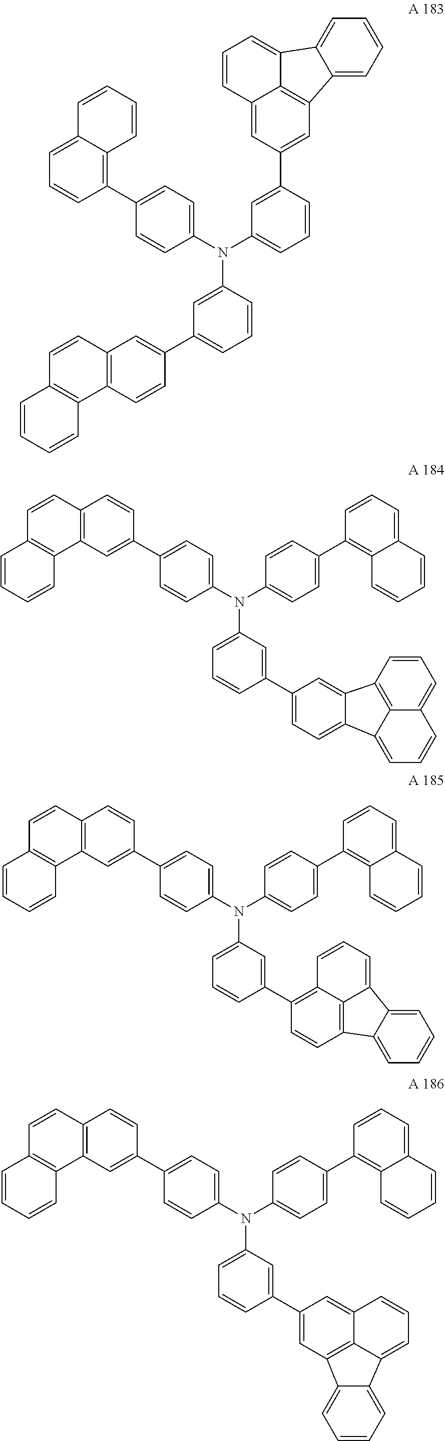

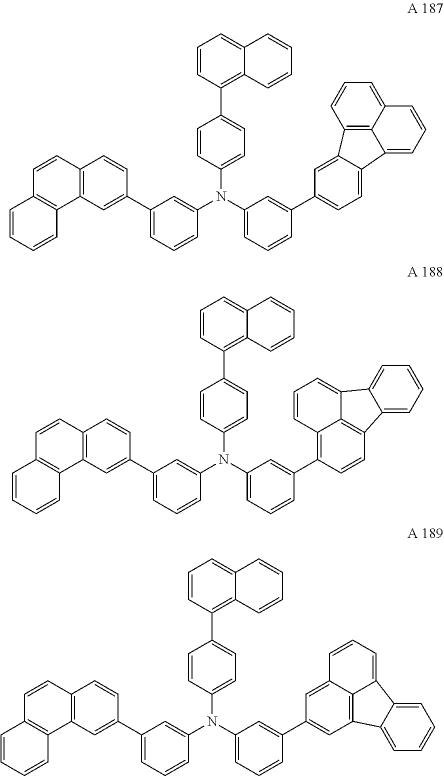 [SMILES: c1cc(-c2ccc3c(c2)-c2cccc4cccc-3c24)cc(N(c2ccc(-c3cccc4ccccc34)cc2)c2cccc(-c3ccc4ccc5ccccc5c4c3)c2)c1 ; c1cc(-c2ccc3ccc4ccccc4c3c2)cc(N(c2ccc(-c3cccc4ccccc34)cc2)c2cccc(-c3ccc4c5c(cccc35)-c3ccccc3-4)c2)c1 ; c1cc(-c2cc3c4c(cccc4c2)-c2ccccc2-3)cc(N(c2ccc(-c3cccc4ccccc34)cc2)c2cccc(-c3ccc4ccc5ccccc5c4c3)c2)c1]

Another one of the objectives of the present invention is to provide an organic electroluminescent device including the above compound.

The material of the present invention is used as a hole transport material in the organic electroluminescent device; or
the material of the present invention is used as an electron blocking layer material in the organic electroluminescent device.

The material of the present invention has the advantages of good film forming property, great optical, electrical, and thermal stability, high luminescence efficiency, low electric voltage, and long service life, and can be used in organic electroluminescent devices. In particular, the compound has the potential for application in the AMOLED industry as a hole transport material or an electron blocking layer material.

### DETAILED DESCRIPTION OF EMBODIMENTS

The following embodiments are merely described to facilitate the understanding of the technical invention, and should not be considered as specific limitations of the present invention.

All raw materials, solvents and the like involved in the synthesis of compounds in the present invention were purchased from Alfa, Acros, and other suppliers known to persons skilled in the art.

### Example 1: Synthesis of a Compound A1

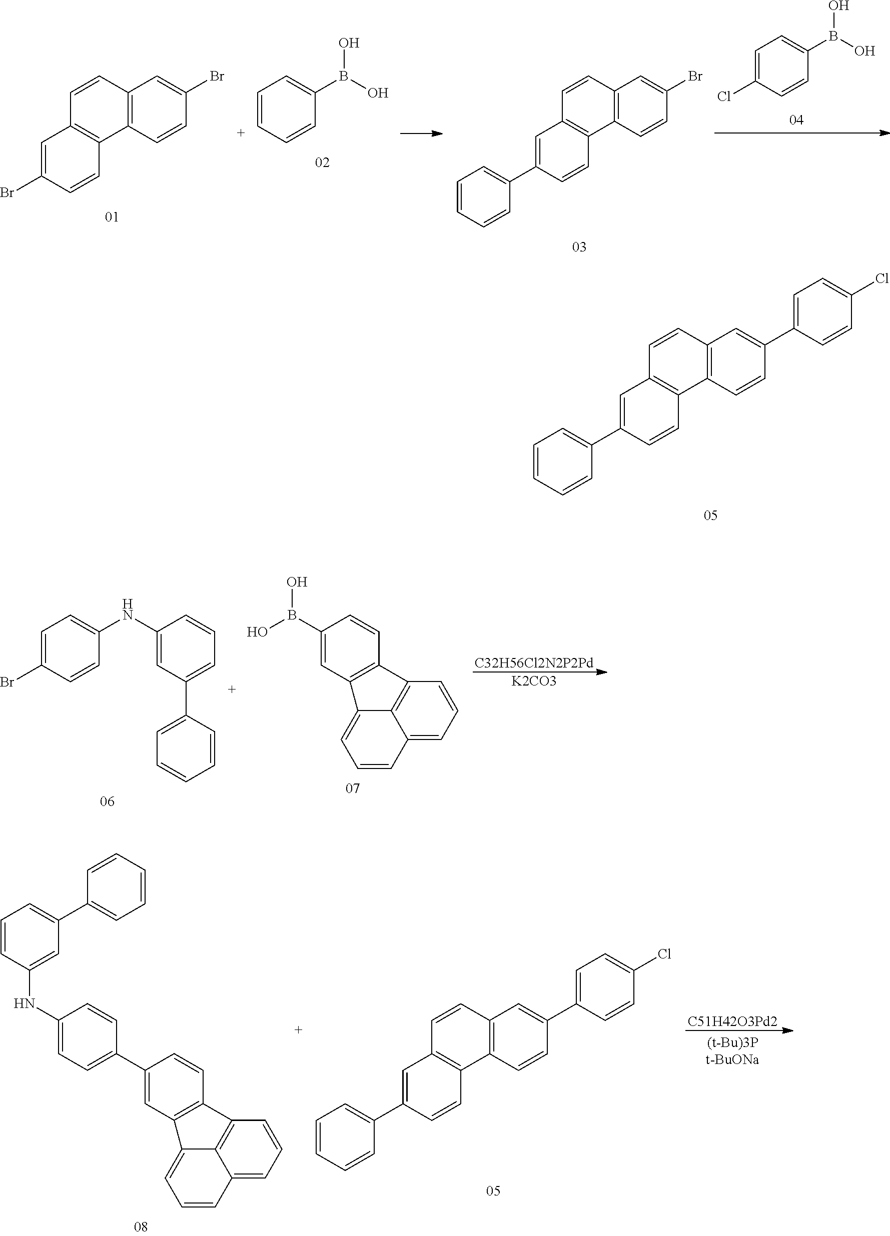

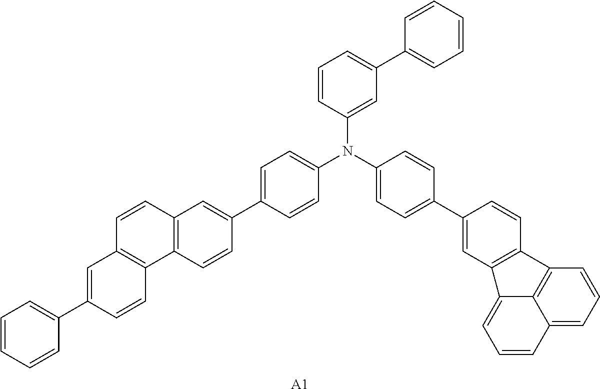 [SMILES: c1ccc(-c2cccc(N(c3ccc(-c4ccc5c(c4)-c4cccc6cccc-5c46)cc3)c3ccc(-c4ccc5c(ccc6cc(-c7ccccc7)ccc65)c4)cc3)c2)cc1 ; "A1"]

Synthesis of a compound 03: A compound 01 (45 g, 0.13 mol, 1.0 eq), a compound 02 (16.3 g, 0.13 mol, 1.0 eq), Pd(PPh₃)₄ (3.1 g, 2.68 mol, 0.02 eq), K₂CO₃ (37.02 g, 0.26 mol, 2.0 eq), and a mixed solvent of THF and H₂O (at a ratio of 8:2, 450 ml in total) were sequentially added to a 1 L three-mouth flask, and stirred under the replacement of vacuum and N₂ for 3 times. A mixture obtained was heated and stirred at about 70° C. for 5 hours. The raw material 01 was monitored by TLC (with Hex as a developing agent) to have a complete reaction. After cooling was conducted, toluene (300 ml) was added, and stirred for 0.5 hour. An organic phase was collected after extraction and liquid separation. A solvent was removed by concentration. Separation was conducted by column chromatography (with Hex as an eluent), and then drying was conducted to obtain 22.09 g of a white solid compound 03 with a yield of 49.5%. Mass spectrometry was as follows: 334.22 (M+H).

Synthesis of a compound 05: The compound 03 (22 g, 66.02 mmol, 1.0 eq), a compound 04 (10.53 g, 67.34 mol, 1.02 eq), Pd(dppf)Cl₂ (0.966 g, 1.32 mmol, 0.02 eq), K₂CO₃ (18.25 g, 132.04 mmol, 2.0 eq), and a mixed solvent of 1,4-dioxane and H₂O (at a ratio of 10:2, 264 ml in total) were sequentially added to a 1 L three-mouth flask, and stirred under the replacement of vacuum and N₂ for 3 times. A mixture obtained was heated to 80° C. for a reaction for 8 hours. The raw material 03 was monitored by TLC (with a mixture of DCM and Hex at a ratio of 1:20 as a developing agent) to have a complete reaction. After a reaction solution was cooled to room temperature, toluene (200 ml) was added, and stirred for 0.5 hour. An organic phase was collected after extraction and liquid separation, and then filtered with diatomite. A filter cake was rinsed with a small amount of toluene, and a filtrate was collected. The organic phase was concentrated to about 150 ml, and cooled to room temperature. Methanol (250 ml) was slowly added, and stirred for crystallization for 3 hours. After filtration was conducted, a filtrate cake was rinsed with a small amount of methanol. A solid was collected, and dried under vacuum at 60° C. for 8 hours to obtain 19.78 g of a white-like solid compound 05 with a yield of 82.1%. Mass spectrometry was as follows: 365.87 (M+H).

Synthesis of a compound 08: A compound 06 (26.8 g, 82.66 mmol, 1.0 eq), a compound 07 (20.34 g, 82.66 mmol, 1.0 eq), Pd₁₃₂ (585.3 mg, 0.826 mmol, 0.01 eq), K₂CO₃ (22.85 g, 165.32 mmol, 2.0 eq), and a mixed solvent of toluene, ethanol, and water (at a ratio of 10:2:2, 375 ml in total) were sequentially added to a 1 L three-mouth flask, and stirred under the replacement of vacuum and N₂ for 3 times. A mixture obtained was heated for reflux for 16 hours. The raw material 06 was monitored by TLC (with a mixture of DCM and Hex at a ratio of 1:5 as a developing agent) to have a complete reaction. After cooling was conducted to room temperature, the mixture was filtered. A filtrate cake was rinsed with ethanol (100 ml), and dried. The filtrate cake was added to a 1 L one-mouth flask, and DCM (600 ml) was added for stirring and dissolution. A mixture obtained was filtered with diatomite, and spin-dried. A solid obtained was beaten with DCM (150 ml) for 2 times, and then dried under vacuum at 70° C. to obtain 27.3 g of a white-like solid compound 08 with a yield of 74.2%. Mass spectrometry was as follows: 446.55 (M+H).

Synthesis of a compound A1: The compound 08 (15 g, 33.67 mmol, 1.0 eq), the compound 05 (12.28 g, 33.67 mmol, 1.0 eq), Pd₂(dba)₃ (924.8 mg, 1.01 mmol, 0.03 eq), a 50% P(t-Bu)₃-containing toluene solution (1.63 g, 2.02 mmol, 0.06 eq), t-BuONa (4.85 g, 50.5 mmol, 1.5 eq), and dried xylene (200 ml) were sequentially added to a 1 L three-mouth flask, and stirred under the replacement of vacuum and N₂ for 3 times. A mixture obtained was heated for reflux for 16 hours. The raw material 05 was monitored by TLC (with a mixture of DCM and Hex at a ratio of 1:8 as a developing agent) to have a complete reaction. After cooling was conducted to room temperature, methanol (150 ml) was added to a reaction solution, and stirred for 2 hours. After suction filtration was conducted, a solid was collected. The solid was added to a 1 L one-mouth flask, and DCM (450 ml) was added for stirring and dissolution. Deionized water was added for water washing and liquid separation for 3 times (150 ml each time). An organic phase was collected, and filtered with silica gel. A filtrate was spin-dried. A solid obtained was heated and dissolved in THF (180 ml). After cooling was conducted, methanol (180 ml) was slowly dropped, and stirred for crystallization for 2 hours. After suction filtration was conducted, a solid was obtained. Recrystallization was conducted for 2 times according to the method, and drying was conducted under vacuum at 70° C. to obtain 16.81 g of a white solid compound A1 with a yield of 64.5%. 16.81 g of the crude product A1 was sublimated and purified to obtain 11.5 g of a sublimated product A1 with a yield of 68.7%. Mass spectrometry was as follows: 774.96 (M+H). ¹H NMR (400 MHz, CDCl₃) δ9.11 (d, 2H), 8.78 (d, 1H), 8.43 (d, J=4.0 Hz, 3H), 7.92 (d, 2H), 7.75 (t, J=27.5 Hz, 8H), 7.62 (d, 2H), 7.45 (m, J=65.0, 25.0 Hz, 17H), 7.27 (t, 1H), 7.17 (m, J=5.0 Hz, 1H), 7.06 (d, 1H).

### Example 2: Synthesis of a Compound A2

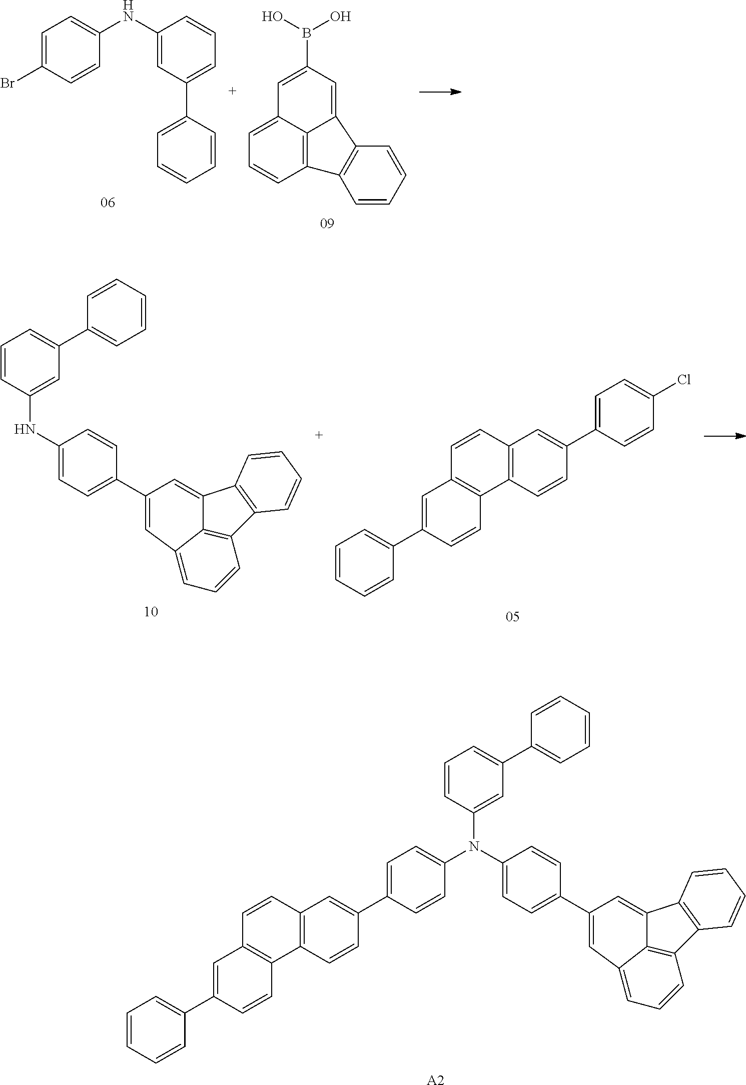

Synthesis of a compound 10: With reference to the synthesis process and post-treatment and purification methods of the compound 08, only the corresponding raw materials were required to be changed. Mass spectrometry was as follows: 446.55 (M+H).

Synthesis of a compound A2: With reference to the synthesis process and post-treatment and purification methods of the compound A1, only the corresponding raw materials were required to be changed, and 17.6 g of a white solid compound A2 with a yield of 67.8% was obtained. 17.6 g of the crude product A2 was sublimated and purified to obtain 12.2 g of a sublimated product A2 with a yield of 69.3%. Mass spectrometry was as follows: 774.96 (M+H). ¹H NMR (400 MHz, CDCl₃) δ 9.11 (d, 2H), 8.54 (d, 1H), 8.43 (m, J=4.0 Hz, 6H), 8.10 (m, 2H), 7.92 (d, 2H), 7.75 (m, J=27.5 Hz, 5H), 7.62 (m, 2H), 7.45 (m, J=65.0, 25.0 Hz, 16H), 7.27 (d, 1H), 7.17 (d, J=5.0 Hz, 2H).

### Example 3: Synthesis of a Compound A4

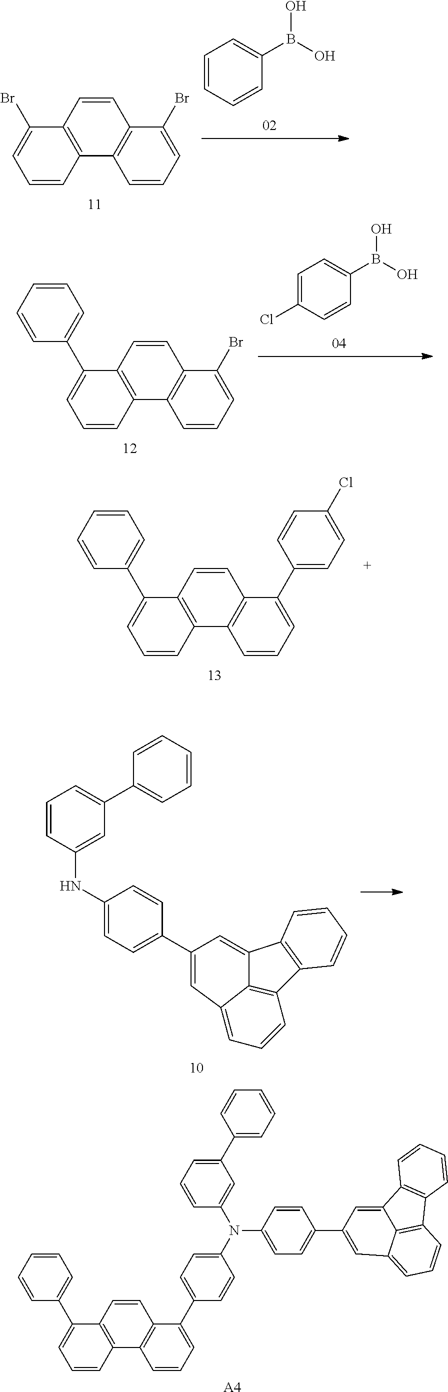

Synthesis of a compound 12: With reference to the synthesis process and post-treatment and purification methods of the compound 03, only the corresponding raw materials were required to be changed. Mass spectrometry was as follows: 334.22 (M+H).

Synthesis of a compound 13: With reference to the synthesis process and post-treatment and purification methods of the compound 05, only the corresponding raw materials were required to be changed. Mass spectrometry was as follows: 365.87 (M+H).

Synthesis of a compound A4: With reference to the synthesis process and post-treatment and purification methods of the compound A1, only the corresponding raw materials were required to be changed, and 15.1 g of a white solid compound A4 with a yield of 62.1% was obtained. 15.1 g of the crude product A4 was sublimated and purified to obtain 9.87 g of a sublimated product A4 with a yield of 65.36%. Mass spectrometry was as follows: 774.96 (M+H). ¹H NMR (400 MHz, CDCl₃) δ 9.08 (dd, 2H), 8.42 (m, 2H), 8.29 (t, 2H), 8.21 (dd, 2H), 8.10 (m, 2H), 7.88-7.71 (m, 6H), 7.68 (d, J=15.0 Hz, 3H), 7.62-7.32 (m, 16H), 7.27 (d, 2H), 7.17 (m, J=5.0 Hz, 2H).

### Example 4: Synthesis of a Compound A21

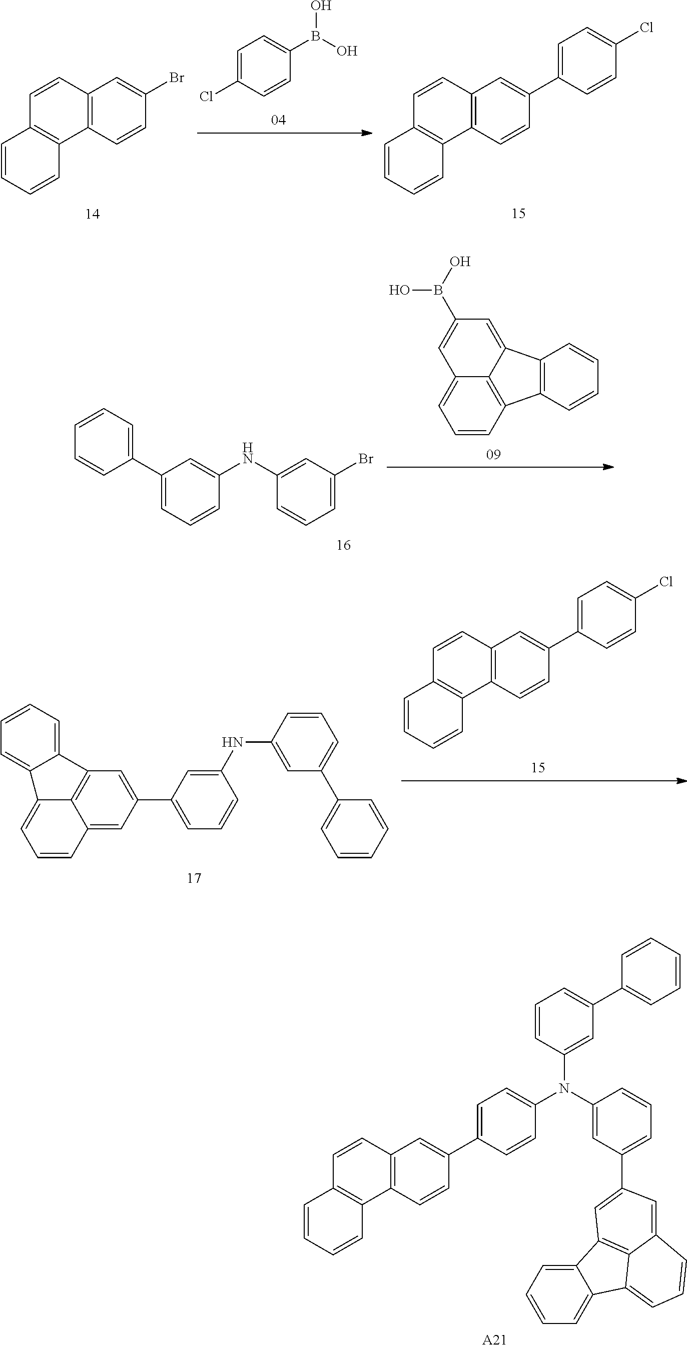

Synthesis of a compound 15: With reference to the synthesis process and post-treatment and purification methods of the compound 05, only the corresponding raw materials were required to be changed. Mass spectrometry was as follows: 289.8 (M+H).

Synthesis of a compound 17: With reference to the synthesis process and post-treatment and purification methods of the compound 08, only the corresponding raw materials were required to be changed. Mass spectrometry was as follows: 446.6 (M+H).

Synthesis of a compound A21: With reference to the synthesis process and post-treatment and purification methods of the compound A1, only the corresponding raw materials were required to be changed, and 13.2 g of a white solid compound A21 with a yield of 64.8% was obtained. 13.2 g of the crude product A21 was sublimated and purified to obtain 8.8 g of a sublimated product A21 with a yield of 66.6%. Mass spectrometry was as follows: 698.9 (M+H). ¹H NMR (400 MHz, CDCl₃) δ 9.11 (d, 1H), 8.70 (dd, 1H), 8.55-8.31 (m, 6H), 8.10 (m, 2H), 7.91 (m, J=10.0 Hz, 2H), 7.86-7.31 (m, 17H), 7.27 (t, 2H), 7.17 (dd, J=5.0 Hz, 4H).

### Example 5: Synthesis of a Compound A24

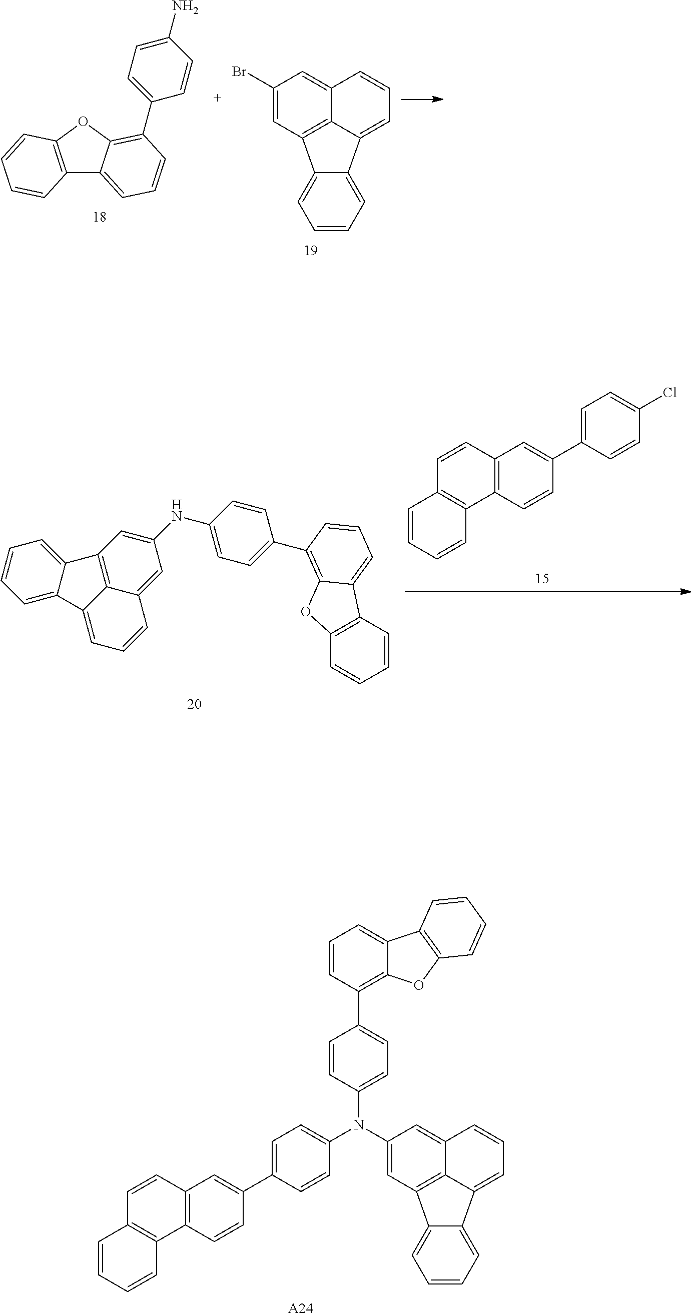

Synthesis of a compound 20: A compound 18 (17.43 g, 67.22 mmol, 1.05 eq), a compound 19 (18 g, 64.02 mmol, 1.0 eq), Pd₂(dba)₃ (1.17 g, 1.28 mmol, 0.02 eq), a 50% P(t-Bu)₃-containing toluene solution (1.04 g, 2.56 mmol, 0.04 eq), t-BuONa (9.23 g, 96.04 mmol, 1.5 eq), and dried xylene (150 ml) were sequentially added to a 500 ml three-mouth flask, and stirred under the replacement of vacuum and N₂ for 3 times. A mixture obtained was heated to 105° C. for a reaction for 6 hours. The raw material 19 was monitored by TLC (with a mixture of DCM and Hex at a ratio of 1:5 as a developing agent) to have a complete reaction. After cooling was conducted to room temperature, toluene (150 ml) was added to a reaction solution, and continuously stirred for 1 hour until the solution was clear. The reaction solution was filtered with silica gel, and rinsed with a small amount of toluene. A filtrate was collected. An organic phase was concentrated to about 150 ml, and cooled to room temperature. Methanol (200 ml) was slowly added, and stirred for crystallization for 2 hours. After filtration was conducted, a filtrate cake was rinsed with a small amount of methanol. A solid obtained was heated and dissolved in THF (180 ml). After cooling was conducted, methanol (180 ml) was slowly dropped, and stirred for crystallization for 2 hours. After suction filtration was conducted, a solid was obtained. The solid was dried under vacuum at 70° C. to obtain 20.07 g of a light yellow solid compound 20 with a yield of 68.2%. Mass spectrometry was as follows: 460.5 (M+H).

Synthesis of a compound A24: With reference to the synthesis process and post-treatment and purification methods of the compound A1, only the corresponding raw materials were required to be changed, and 12.4 g of a white solid compound A24 with a yield of 63.03% was obtained. 12.4 g of the crude product A24 was sublimated and purified to obtain 9.3 g of a sublimated product A24 with a yield of 75%. Mass spectrometry was as follows: 712.8 (M+H). ¹H NMR (400 MHz, CDCl₃) δ 9.11 (d, 1H), 8.70 (dd, 1H), 8.50-8.35 (m, 6H), 8.15-7.87 (m, 7H), 7.82-7.50 (m, 11H), 7.39 (t, J=10.0 Hz, 7H), 7.30 (d, J=15.0 Hz, 2H).

### Example 6: Synthesis of a Compound A27

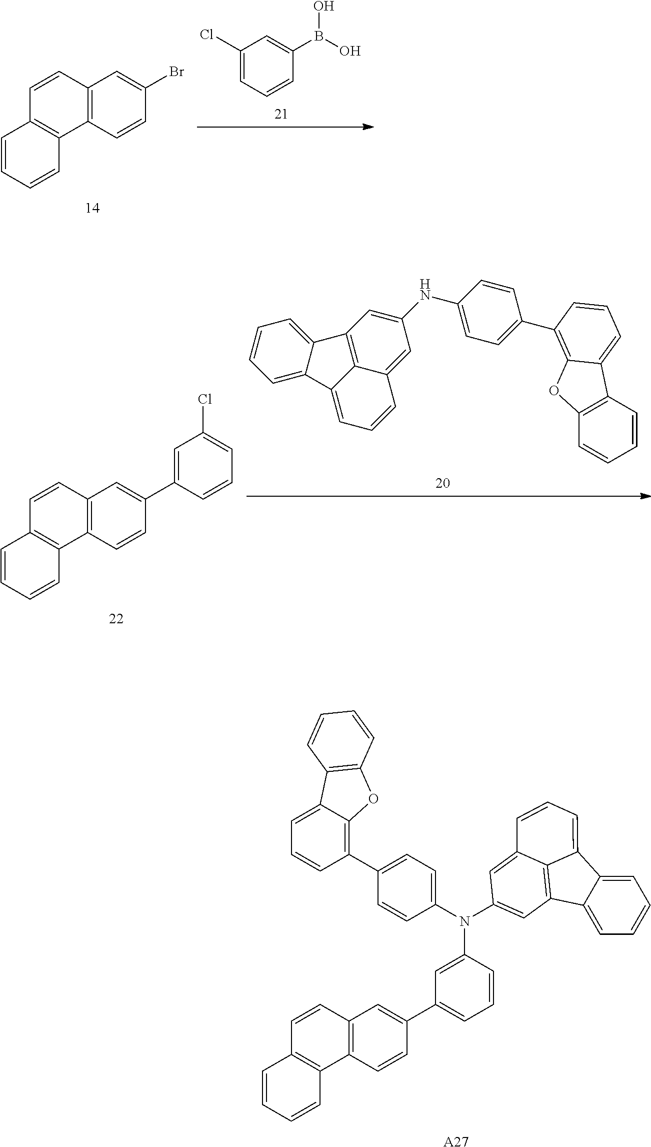 [SMILES: Brc1ccc2c(ccc3ccccc32)c1 ; OB(O)c1cccc(Cl)c1 ; Clc1cccc(-c2ccc3c(ccc4ccccc43)c2)c1 ; c1ccc2c(c1)-c1cccc3cc(Nc4ccc(-c5cccc6c5oc5ccccc56)cc4)cc-2c13 ; c1cc(-c2ccc3c(ccc4ccccc43)c2)cc(N(c2ccc(-c3cccc4c3oc3ccccc34)cc2)c2cc3c4c(cccc4c2)-c2ccccc2-3)c1 ; "A27"]

Synthesis of a compound 22: With reference to the synthesis process and post-treatment and purification methods of the compound 05, only the corresponding raw materials were required to be changed. Mass spectrometry was as follows: 289.8 (M+H).

Synthesis of a compound A27: With reference to the synthesis process and post-treatment and purification methods of the compound A1, only the corresponding raw materials were required to be changed, and 14.2 g of a white solid compound A27 with a yield of 58.9% was obtained. 14.2 g of the crude product A27 was sublimated and purified to obtain 9.5 g of a sublimated product A27 with a yield of 66.9%. Mass spectrometry was as follows: 712.8 (M+H). ¹H NMR (400 MHz, CDCl₃) δ 9.11 (d, 1H), 8.70 (dd, 1H), 8.53-8.35 (m, 5H), 8.18-7.86 (m, 7H), 7.84-7.49 (m, 10H), 7.46-7.32 (m, 4H), 7.33-7.23 (m, 3H), 7.17 (d, J=5.0 Hz, 2H).

### Example 7: Synthesis of a Compound A33

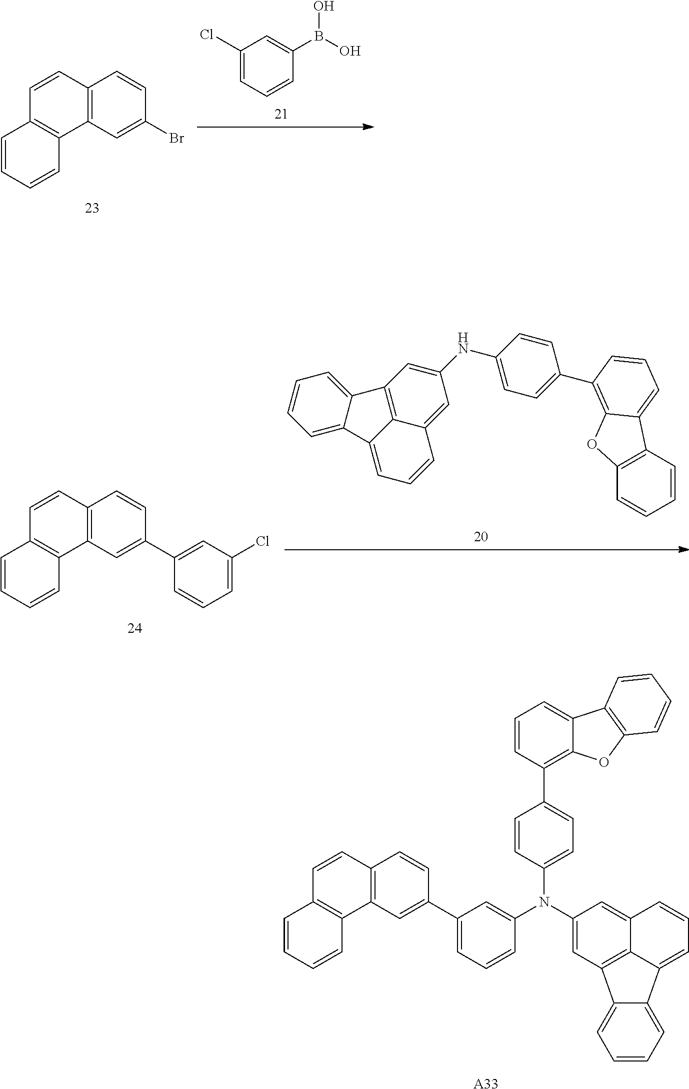 [SMILES: Brc1ccc2ccc3ccccc3c2c1 ; OB(O)c1cccc(Cl)c1 ; Clc1cccc(-c2ccc3ccc4ccccc4c3c2)c1 ; c1ccc2c(c1)-c1cccc3cc(Nc4ccc(-c5cccc6c5oc5ccccc56)cc4)cc-2c13 ; c1cc(-c2ccc3ccc4ccccc4c3c2)cc(N(c2ccc(-c3cccc4c3oc3ccccc34)cc2)c2cc3c4c(cccc4c2)-c2ccccc2-3)c1 ; "A33"]

Synthesis of a compound 24: With reference to the synthesis process and post-treatment and purification methods of the compound 05, only the corresponding raw materials were required to be changed. Mass spectrometry was as follows: 289.8 (M+H).

Synthesis of a compound A33: With reference to the synthesis process and post-treatment and purification methods of the compound A1, only the corresponding raw materials were required to be changed, and 12.5 g of a white solid compound A33 with a yield of 57.9% was obtained. 12.5 g of the crude product A33 was sublimated and purified to obtain 7.9 g of a sublimated product A33 with a yield of 63.2%. Mass spectrometry was as follows: 712.8 (M+H). ¹H NMR (400 MHz, CDCl₃) δ 9.11 (d, 1H), 8.85 (dd, 1H), 8.39 (m, J=27.6, 7.4 Hz, 5H), 8.05 (m J=45.0, 15.0 Hz, 5H), 7.90 (dd, 1H), 7.77 (d, J=22.0 Hz, 3H), 7.73-7.47 (m, 8H), 7.46-7.33 (m, 5H), 7.32-7.22 (m, 3H), 7.18 (d, J=5.0 Hz, 2H).

Example 8: Synthesis of a compound A70

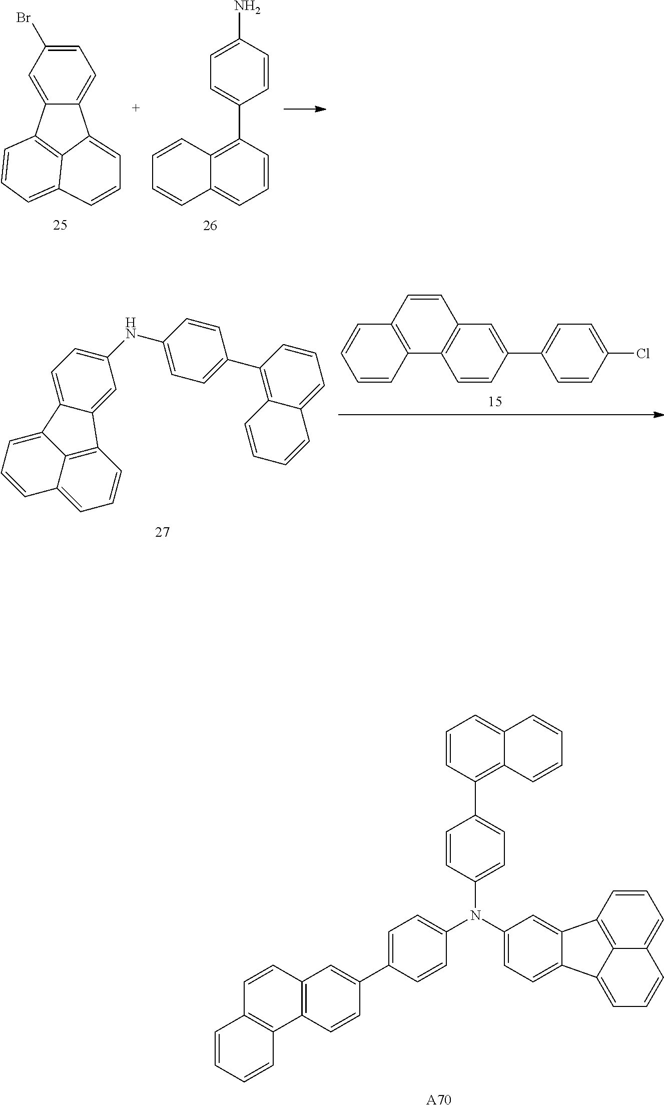

Synthesis of a compound 27: With reference to the synthesis process and post-treatment and purification methods of the compound 20, only the corresponding raw materials were required to be changed. Mass spectrometry was as follows: 419.5 (M+H).

Synthesis of a compound A70: With reference to the synthesis process and post-treatment and purification methods of the compound A1, only the corresponding raw materials were required to be changed, and 9.9 g of a white solid compound A70 with a yield of 54.7% was obtained. 9.9 g of the crude product A70 was sublimated and purified to obtain 6.8 g of a sublimated product A70 with a yield of 66.6%. Mass spectrometry was as follows: 672.8 (M+H). ¹H NMR (400 MHz, CDCl₃) δ 9.11 (d, 1H), 8.95 (dd, 1H), 8.70 (dd, 1H), 8.50 (m, 1H), 8.42 (m, J=13.0 Hz, 2H), 7.90 (t, J=7.5 Hz, 3H), 7.86-7.61 (m, 9H), 7.55 (m, 6H), 7.38 (m, J=20.0, 10.0 Hz, 7H), 7.18 (dd, 1H), 6.93 (d, 1H).

Example 9: Synthesis of a compound A72

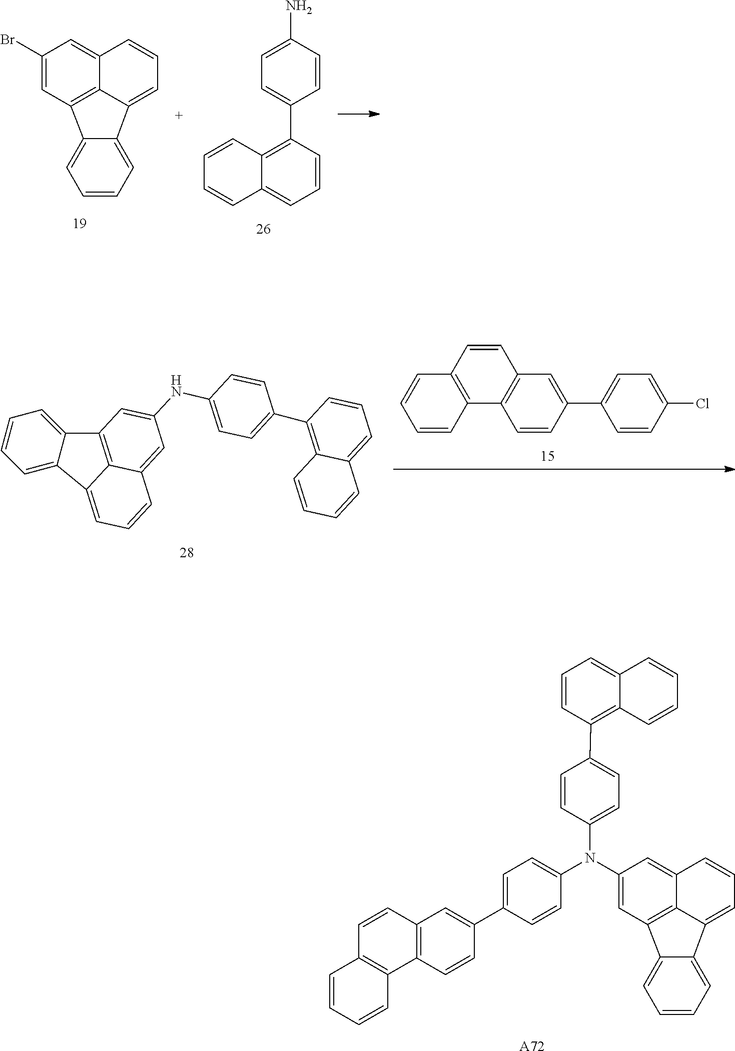

Synthesis of a compound A2: With reference to the synthesis process and post-treatment and purification methods of the compound 20, only the corresponding raw materials were required to be changed. Mass spectrometry was as follows: 419.5 (M+H).

Synthesis of a compound A72: With reference to the synthesis process and post-treatment and purification methods of the compound A1, only the corresponding raw materials were required to be changed, and 12.0 g of a white solid compound A72 with a yield of 63.8% was obtained. 12.0 g of the crude product A72 was sublimated and purified to obtain 8.7 g of a sublimated product A72 with a yield of 72.5%. Mass spectrometry was as follows: 672.8 (M+H). ¹H NMR (400 MHz, CDCl₃) δ 9.11 (d, 1H), 8.95 (dd, 1H), 8.70 (dd, 1H), 8.50 (m, 1H), 8.46-8.37 (m, 5H), 8.10 (m, 2H), 7.90 (m, J=7.5 Hz, 3H), 7.82-7.58 (m, 7H), 7.55 (m, J=5.0 Hz, 5H), 7.35 (m, J=37.5, 22.5 Hz, 7H).

Example 10: Synthesis of a compound A81

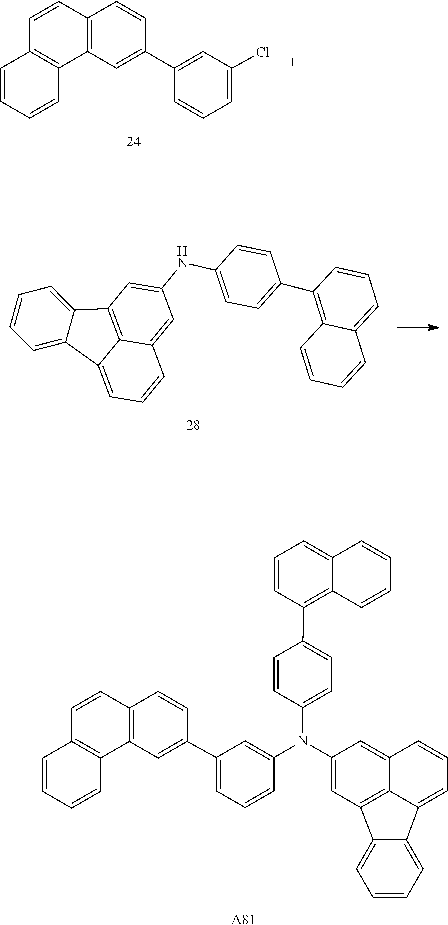 [SMILES: Clc1cccc(-c2ccc3ccc4ccccc4c3c2)c1 ; c1ccc2c(c1)-c1cccc3cc(Nc4ccc(-c5cccc6ccccc56)cc4)cc-2c13 ; c1cc(-c2ccc3ccc4ccccc4c3c2)cc(N(c2ccc(-c3cccc4ccccc34)cc2)c2cc3c4c(cccc4c2)-c2ccccc2-3)c1 ; "A81"]

Synthesis of a compound A81: With reference to the synthesis process and post-treatment and purification methods of the compound A1, only the corresponding raw materials were required to be changed, and 16.6 g of a white solid compound A81 with a yield of 54.3% was obtained. 16.6 g of the crude product A81 was sublimated and purified to obtain 11.9 g of a sublimated product A81 with a yield of 71.6%. Mass spectrometry was as follows: 672.8 (M+H). ¹H NMR (400 MHz, CDCl₃) δ 9.10 (d, 1H), 8.95 (dd, 1H), 8.85 (dd, 1H), 8.50 (m, 1H), 8.39 (m J=30.7, 5.7 Hz, 5H), 8.10 (m, 2H), 7.89 (d, J=5.0 Hz, 2H), 7.77 (m, J=9.1, 5.9 Hz, 5H), 7.70-7.49 (m, 7H), 7.47-7.23 (m, 6H), 7.17 (d, J=5.0 Hz, 2H).

### Example 11: Synthesis of a Compound A118

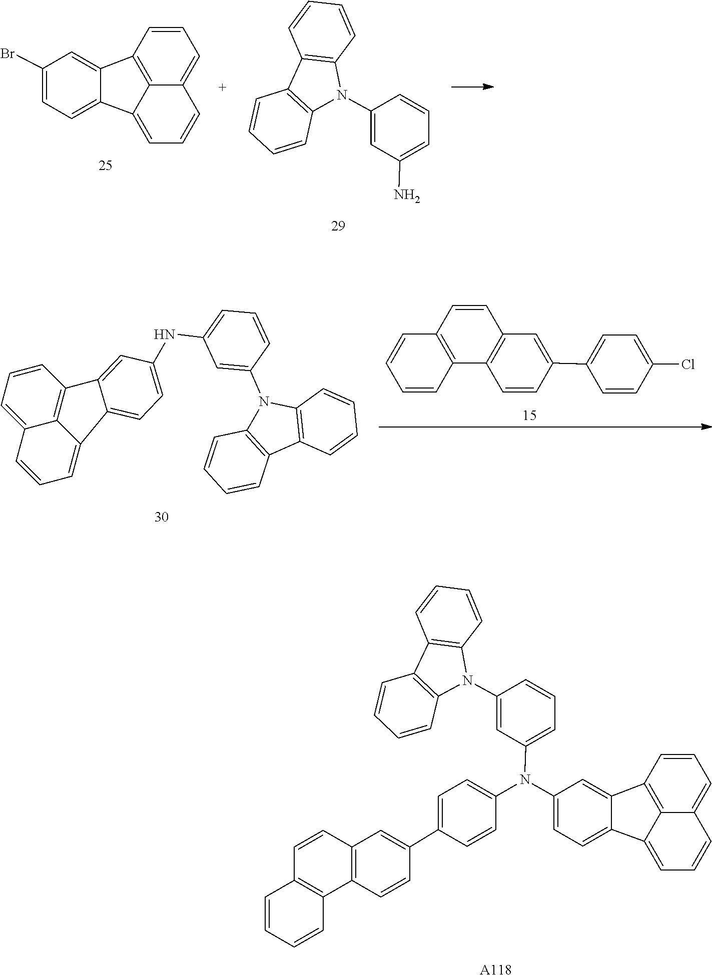 [SMILES: Brc1ccc2c(c1)-c1cccc3cccc-2c13 ; Nc1cccc(-n2c3ccccc3c3ccccc32)c1 ; c1cc(Nc2ccc3c(c2)-c2cccc4cccc-3c24)cc(-n2c3ccccc3c3ccccc32)c1 ; Clc1ccc(-c2ccc3c(ccc4ccccc43)c2)cc1 ; c1cc(N(c2ccc(-c3ccc4c(ccc5ccccc54)c3)cc2)c2ccc3c(c2)-c2cccc4cccc-3c24)cc(-n2c3ccccc3c3ccccc32)c1 ; "A118"]

Synthesis of a compound 30: With reference to the synthesis process and post-treatment and purification methods of the compound 20, only the corresponding raw materials were required to be changed. Mass spectrometry was as follows: 459.6 (M+H).

Synthesis of a compound A118: With reference to the synthesis process and post-treatment and purification methods of the compound A1, only the corresponding raw materials were required to be changed, and 9.3 g of a white solid compound A118 with a yield of 61.2% was obtained. 9.3 g of the crude product A118 was sublimated and purified to obtain 6.7 g of a sublimated product A118 with a yield of 72.1%. Mass spectrometry was as follows: 711.9 (M+H). ¹H NMR (400 MHz, CDCl₃) δ 9.11 (d, 1H), 8.70 (dd, 1H), 8.55 (dd, 1H), 8.45 (m, J=16.1 Hz, 2H), 8.19 (m, 1H), 7.91 (m, J=10.0 Hz, 2H), 7.86-7.47 (m, 13H), 7.39 (m, J=15.0, 10.0 Hz, 5H), 7.16 (m, J=27.5, 17.5 Hz, 6H), 7.04 (m, 1H), 6.93 (d, 1H).

### Example 12: Synthesis of a Compound A138

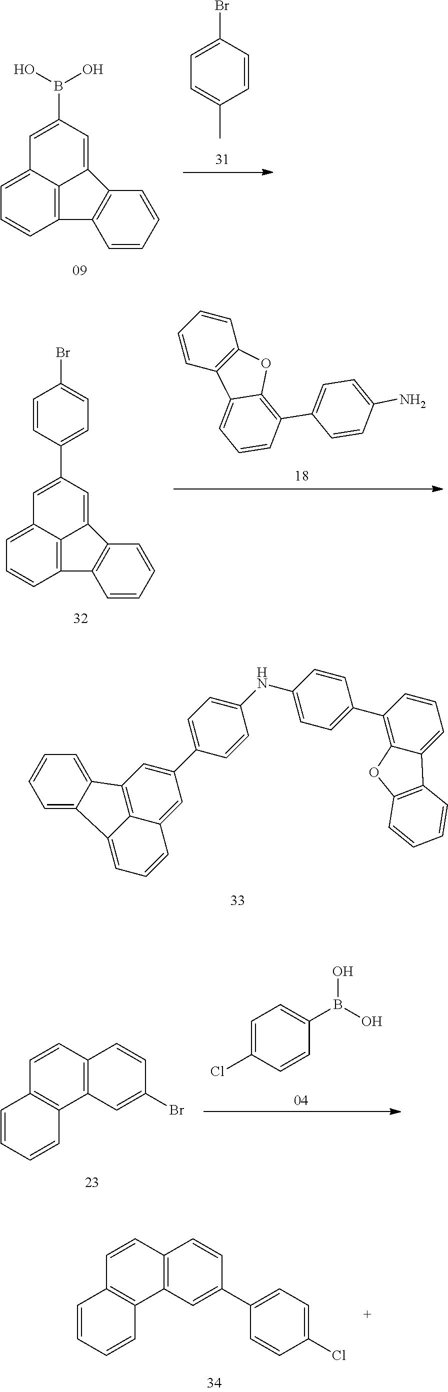

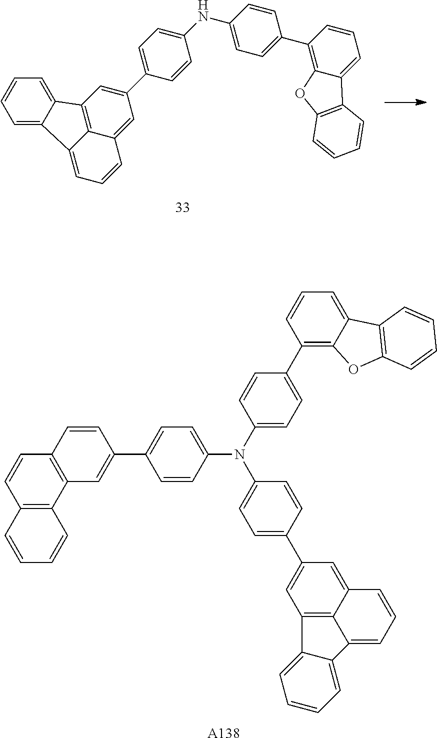 [SMILES: c1ccc2c(c1)-c1cccc3cc(-c4ccc(Nc5ccc(-c6cccc7c6oc6ccccc67)cc5)cc4)cc-2c13 ; c1ccc2c(c1)-c1cccc3cc(-c4ccc(N(c5ccc(-c6ccc7ccc8ccccc8c7c6)cc5)c5ccc(-c6cccc7c6oc6ccccc67)cc5)cc4)cc-2c13 ; "A138"]

Synthesis of a compound 32: A compound 09 (18 g, 73.15 mmol, 1.0 eq), a compound 31 (21.1 g, 74.61 mmol, 1.02 eq), Pd(dppf)Cl₂ (1.07 g, 1.46 mmol, 0.02 eq), K₂CO₃ (20.2 g, 146.3 mmol, 2.0 eq), and a mixed solvent of 1,4-dioxane and H₂O (at a ratio of 10:2, 216 ml in total) were sequentially added to a 1 L three-mouth flask, and stirred under the replacement of vacuum and N₂ for 3 times. A mixture obtained was heated to 70° C. for a reaction for 8 hours. The raw material 09 was monitored by TLC (with a mixture of DCM and Hex at a ratio of 1:20 as a developing agent) to have a complete reaction. After a reaction solution was cooled to room temperature, toluene (100 ml) was added, and stirred for 0.5 hour. An organic phase was collected after extraction and liquid separation, and then filtered with diatomite. A filter cake was rinsed with a small amount of toluene, and a filtrate was collected, concentrated to about 100 ml, and cooled to room temperature. N-hexane (250 ml) was slowly added, and stirred for crystallization for 3 hours. After filtration was conducted, a filtrate cake was rinsed with a small amount of n-hexane. A solid was collected, and dried under vacuum at 60° C. for 8 hours to obtain 18.79 g of a white solid compound 32 with a yield of 71.9%. Mass spectrometry was as follows: 358.2 (M+H).

Synthesis of a compound 33: With reference to the synthesis process and post-treatment and purification methods of the compound 20, only the corresponding raw materials were required to be changed. Mass spectrometry was as follows: 536.5 (M+H).

Synthesis of a compound A138: With reference to the synthesis process and post-treatment and purification methods of the compound A1, only the corresponding raw materials were required to be changed, and 12.3 g of a white solid compound A138 with a yield of 62.1% was obtained. 12.3 g of the crude product A138 was sublimated and purified to obtain 7.9 g of a sublimated product A138 with a yield of 64.2%. Mass spectrometry was as follows: 788.9 (M+H). ¹H NMR (400 MHz, CDCl₃) δ 9.09 (d, 1H), 8.92 (d, 1H), 8.85 (dd, 1H), 8.70 (t, 1H), 8.49-8.30 (m, 4H), 8.05 (m, J=45.0, 15.0 Hz, 5H), 7.90 (s, 1H), 7.81 (dd, 1H), 7.78-7.49 (m, 13H), 7.37 (m, J=30.0, 20.0 Hz, 9H).

### Example 13: Synthesis of a Compound A150

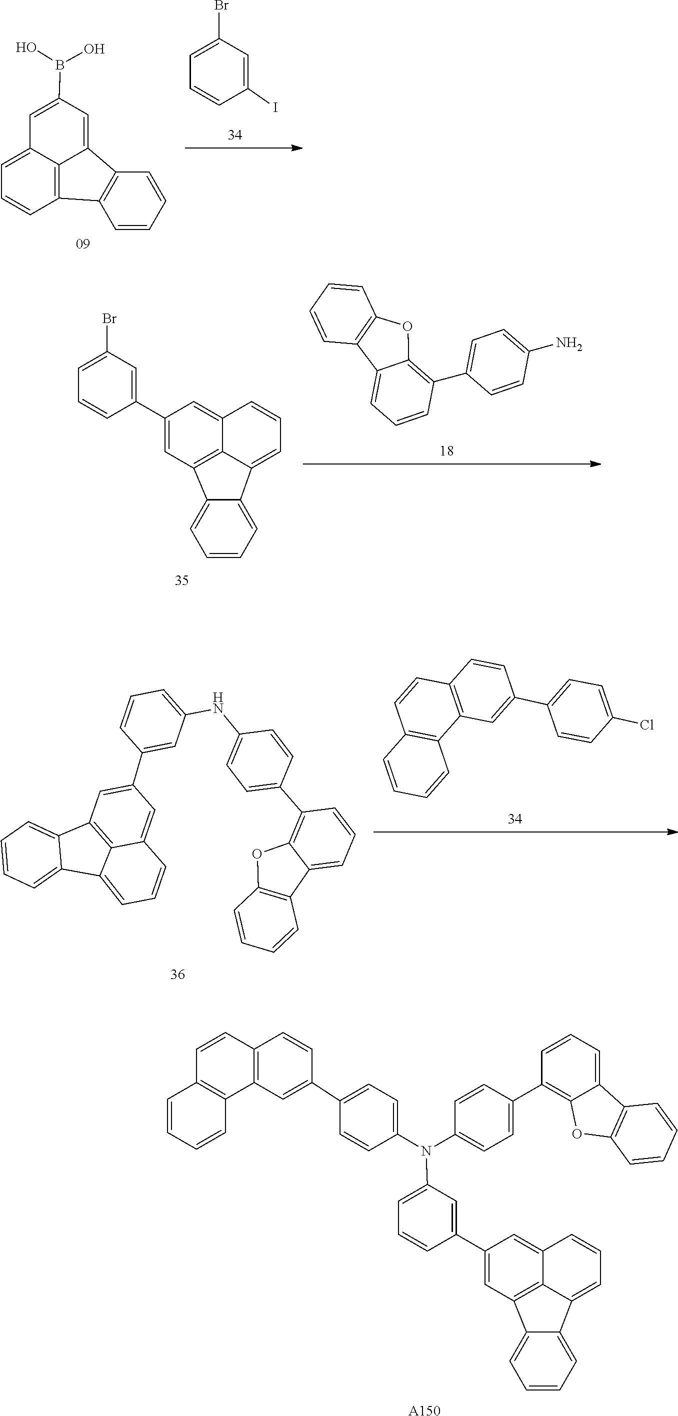

Synthesis of a compound 35: With reference to the synthesis process and post-treatment and purification methods of the compound 32, only the corresponding raw materials were required to be changed. Mass spectrometry was as follows: 358.2 (M+H).

Synthesis of a compound 36: With reference to the synthesis process and post-treatment and purification methods of the compound 20, only the corresponding raw materials were required to be changed. Mass spectrometry was as follows: 536.5 (M+H).

Synthesis of a compound A150: With reference to the synthesis process and post-treatment and purification methods of the compound A1, only the corresponding raw materials were required to be changed, and 10.56 g of a white solid compound A150 with a yield of 77.2% was obtained. 10.56 g of the crude product A150 was sublimated and purified to obtain 6.4 g of a sublimated product A150 with a yield of 60.6%. Mass spectrometry was as follows: 788.9 (M+H). ¹H NMR (400 MHz, CDCl₃) δ 9.05 (d, 1H), 8.85 (dd, 1H), 8.52-8.32 (m, 5H), 8.29 (d, 1H), 8.05 (m, J=45.0, 15.0 Hz, 5H), 7.90 (dd, 1H), 7.81 (dd, 1H), 7.75 (s, 2H), 7.73-7.49 (m, 10H), 7.39 (m, J=10.0 Hz, 6H), 7.29 (m, J=20.0 Hz, 2H), 7.17 (m, J=5.0 Hz, 2H).

### Example 14: Synthesis of a Compound A158

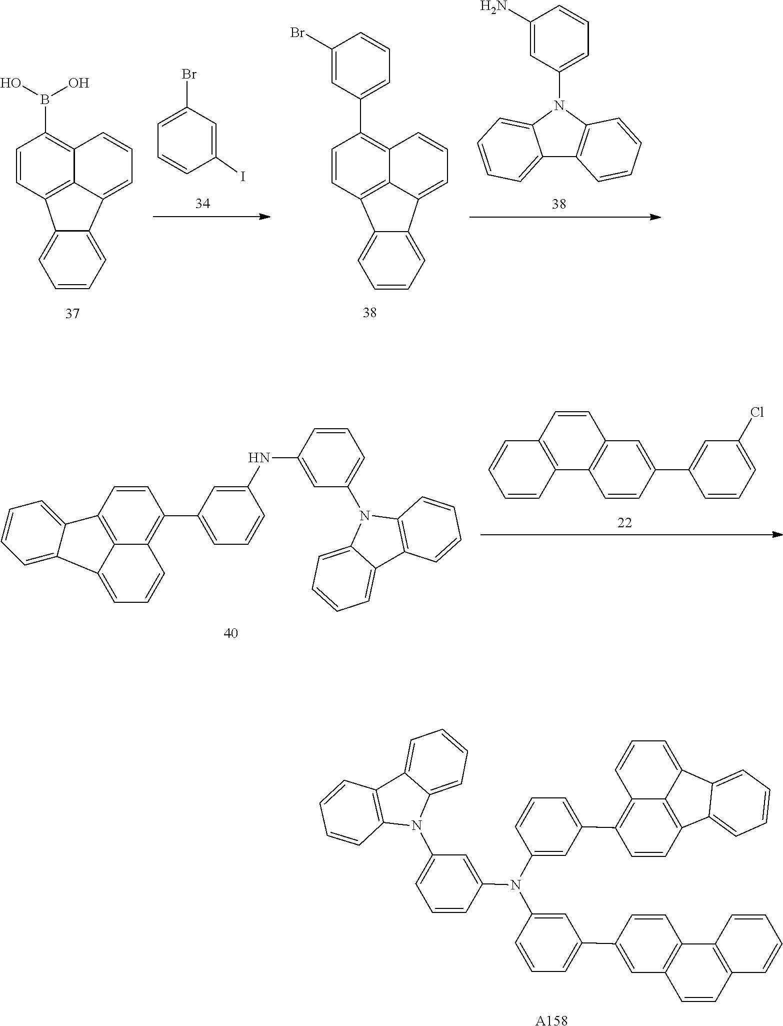

Synthesis of a compound 38: With reference to the synthesis process and post-treatment and purification methods of the compound 32, only the corresponding raw materials were required to be changed. Mass spectrometry was as follows: 358.2 (M+H).

Synthesis of a compound 40: With reference to the synthesis process and post-treatment and purification methods of the compound 20, only the corresponding raw materials were required to be changed. Mass spectrometry was as follows: 535.6 (M+H).

Synthesis of a compound A158: With reference to the synthesis process and post-treatment and purification methods of the compound A1, only the corresponding raw materials were required to be changed, and 6.52 g of a white solid compound A158 with a yield of 68.1% was obtained. 6.52 g of the crude product A158 was sublimated and purified to obtain 4.93 g of a sublimated product A158 with a yield of 75.6%. Mass spectrometry was as follows: 788.0 (M+H). ¹H NMR (400 MHz, CDCl₃) δ 9.11 (d, 1H), 8.68 (m, J=21.6 Hz, 2H), 8.53 (m, J=23.8 Hz, 2H), 8.43 (m, J=5.0 Hz, 3H), 8.19 (m, 1H), 8.10 (m, 2H), 7.91 (m, J=10.0 Hz, 2H), 7.76 (m, J=5.0 Hz, 2H), 7.70-7.57 (m, 5H), 7.57-7.33 (m, 7H), 7.27 (s, 2H), 7.23-7.09 (m, 9H), 7.04 (s, 1H).

### Example 15: Synthesis of a Compound A159

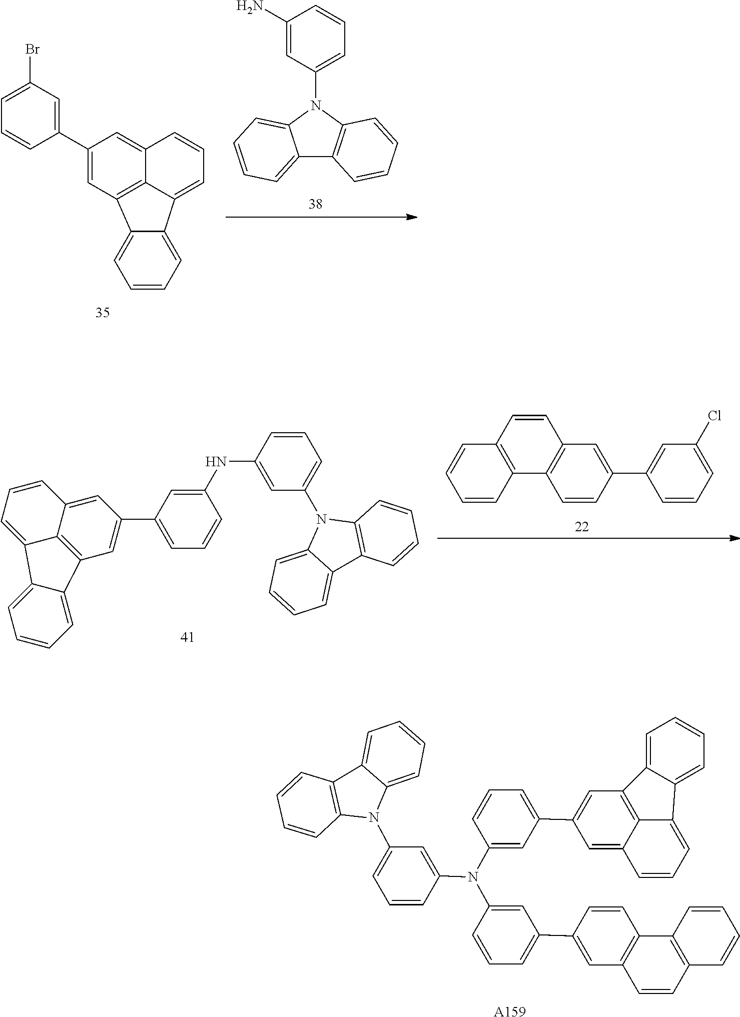

Synthesis of a compound 41: With reference to the synthesis process and post-treatment and purification methods of the compound 20, only the corresponding raw materials were required to be changed. Mass spectrometry was as follows: 535.6 (M+H).

Synthesis of a compound A159: With reference to the synthesis process and post-treatment and purification methods of the compound A1, only the corresponding raw materials were required to be changed, and 6.94 g of a white solid compound A159 with a yield of 65.5% was obtained. 6.94 g of the crude product A159 was sublimated and purified to obtain 5.1 g of a sublimated product A159 with a yield of 73.4%. Mass spectrometry was as follows: 788.0 (M+H). ¹H NMR (400 MHz, CDCl₃) δ 9.11 (d, 1H), 8.70 (dd, 1H), 8.55 (dd, 1H), 8.52-8.35 (m, 7H), 8.19 (m, 1H), 8.10 (m, 2H), 7.91 (m, J=10.0 Hz, 2H), 7.78 (dd, J=30.0 Hz, 2H), 7.72-7.61 (m, 3H), 7.55 (m, J=12.5 Hz, 4H), 7.41 (m, J=10.0 Hz, 2H), 7.27 (t, 2H), 7.24-7.09 (m, 9H), 7.04 (m, 1H).

### Example 16: Synthesis of a Compound A174

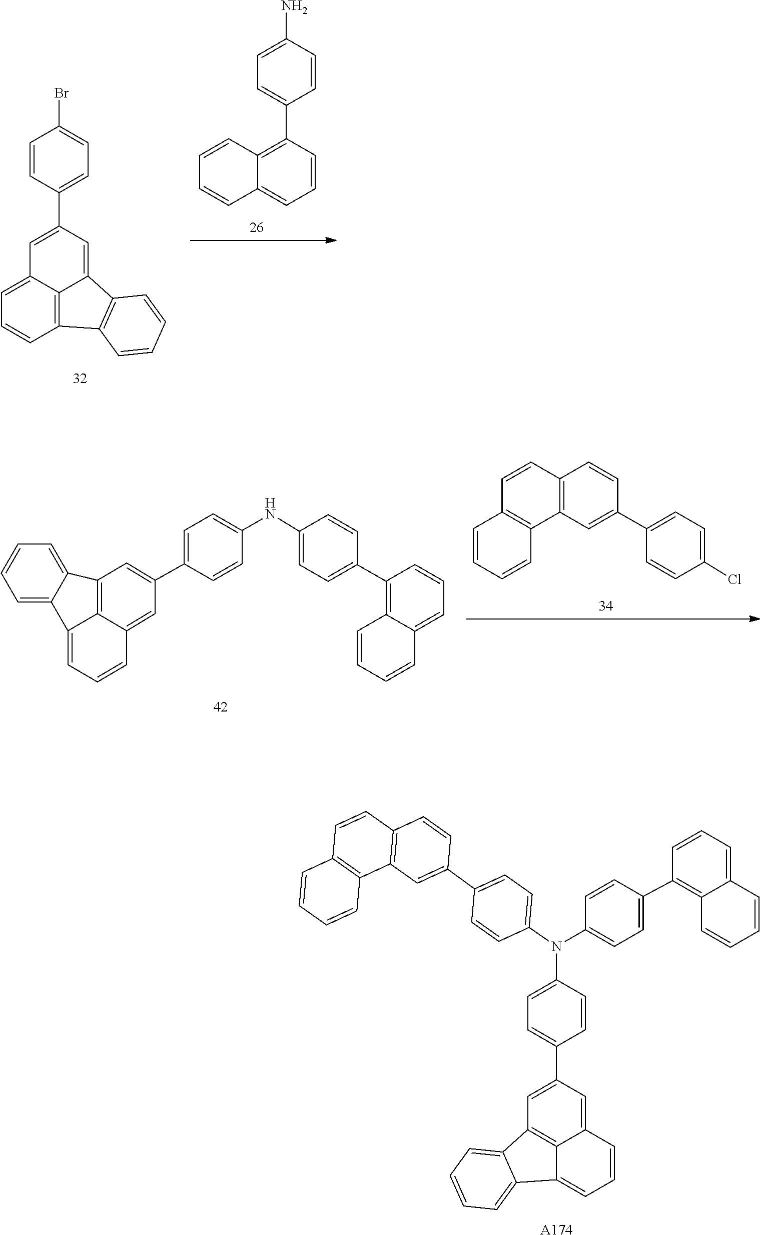

Synthesis of a compound 42: With reference to the synthesis process and post-treatment and purification methods of the compound 20, only the corresponding raw materials were required to be changed. Mass spectrometry was as follows: 496.6 (M+H).

Synthesis of a compound A174: With reference to the synthesis process and post-treatment and purification methods of the compound A1, only the corresponding raw materials were required to be changed, and 7.4 g of a white solid compound A174 with a yield of 71.1% was obtained. 7.4 g of the crude product A174 was sublimated and purified to obtain 5.2 g of a sublimated product A174 with a yield of 70.2%. Mass spectrometry was as follows: 748.9 (M+H). ¹H NMR (400 MHz, CDCl₃) δ 9.40 (t, 1H), 9.07 (d, 1H), 8.95 (dd, 1H), 8.85 (dd, 1H), 8.61 (d, 1H), 8.50 (m, 1H), 8.42 (m, 2H), 8.36 (d, J=10.0 Hz, 2H), 8.10 (m, 2H), 7.89 (d, J=5.0 Hz, 2H), 7.86-7.73 (m, 6H), 7.73-7.49 (m, 10H), 7.47-7.29 (m, 8H).

### Example 17: Synthesis of a Compound A181

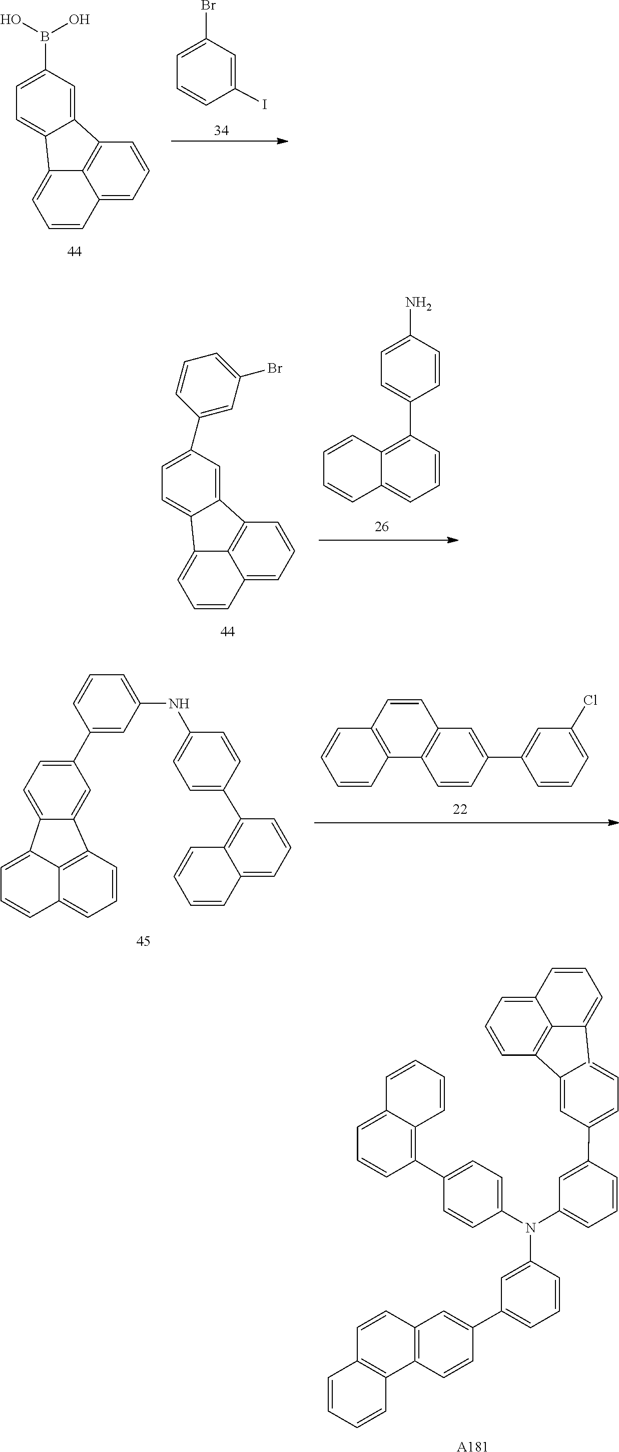

Synthesis of a compound 44: With reference to the synthesis process and post-treatment and purification methods of the compound 32, only the corresponding raw materials were required to be changed. Mass spectrometry was as follows: 358.2 (M+H).

Synthesis of a compound 45: With reference to the synthesis process and post-treatment and purification methods of the compound 20, only the corresponding raw materials were required to be changed. Mass spectrometry was as follows: 496.6 (M+H).

Synthesis of a compound A181: With reference to the synthesis process and post-treatment and purification methods of the compound A1, only the corresponding raw materials were required to be changed, and 6.3 g of a white solid compound A181 with a yield of 62.1% was obtained. 6.3 g of the crude product A181 was sublimated and purified to obtain 4.2 g of a sublimated product A181 with a yield of 66.6%. Mass spectrometry was as follows: 748.9 (M+H). ¹H NMR (400 MHz, CDCl₃) δ 9.11 (d, 1H), 8.95 (dd, 1H), 8.70 (dd, 1H), 8.46 (t, J=17.5 Hz, 3H), 7.90 (m, J=7.5 Hz, 3H), 7.86-7.75 (m, 6H), 7.65 (q, J=5.0 Hz, 6H), 7.55 (s, 6H), 7.47-7.29 (m, 4H), 7.27 (dd, 2H), 7.17 (m, J=5.0 Hz, 4H), 7.06 (dd, 1H).

### Example 18: Synthesis of a Compound A94

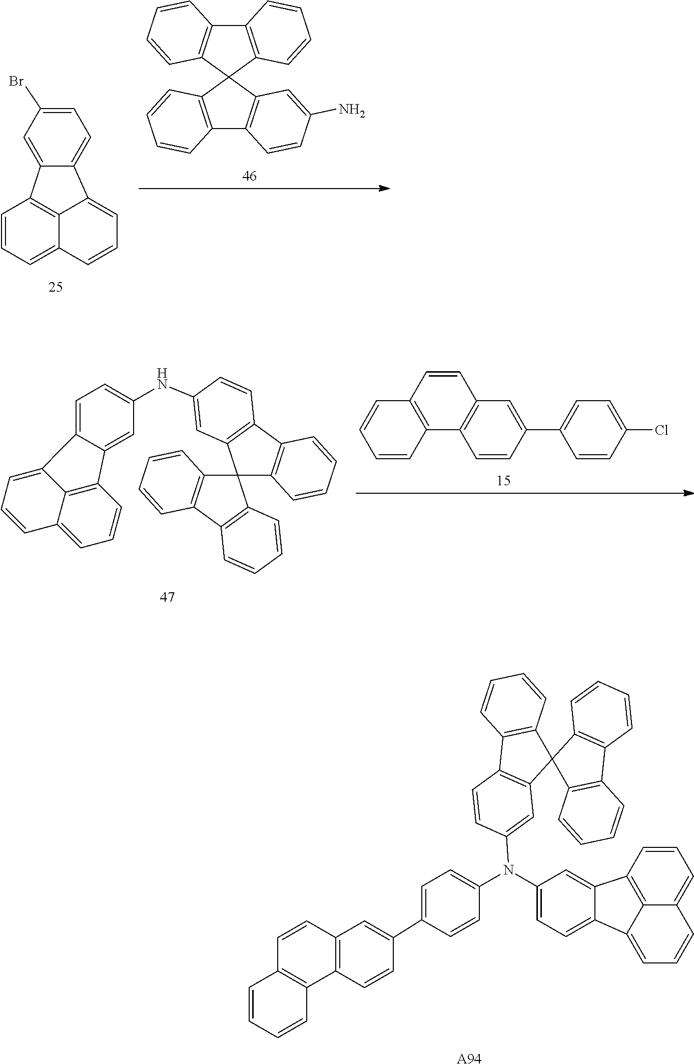

Synthesis of a compound 47: With reference to the synthesis process and post-treatment and purification methods of the compound 20, only the corresponding raw materials were required to be changed. Mass spectrometry was as follows: 532.6 (M+H).

Synthesis of a compound A94: With reference to the synthesis process and post-treatment and purification methods of the compound A1, only the corresponding raw materials were required to be changed, and 6.1 g of a white solid compound A94 with a yield of 57.0% was obtained. 6.1 g of the crude product A94 was sublimated and purified to obtain 4.3 g of a sublimated product A94 with a yield of 70.4%. Mass spectrometry was as follows: 784.9 (M+H). ¹H NMR (400 MHz, CDCl₃) δ 9.11 (d, 1H), 8.70 (dd, 1H), 8.41 (m, J=20.5 Hz, 2H), 7.96-7.75 (m, 9H), 7.75-7.61 (m, 4H), 7.55 (m, 4H), 7.47 (dd, 1H), 7.43-7.29 (m, 4H), 7.30-7.14 (m, 6H), 6.93 (d, 1H), 6.81 (m, 2H), 6.43 (dd, J=11.5 Hz, 2H).

### Example 19: Synthesis of a Compound A96

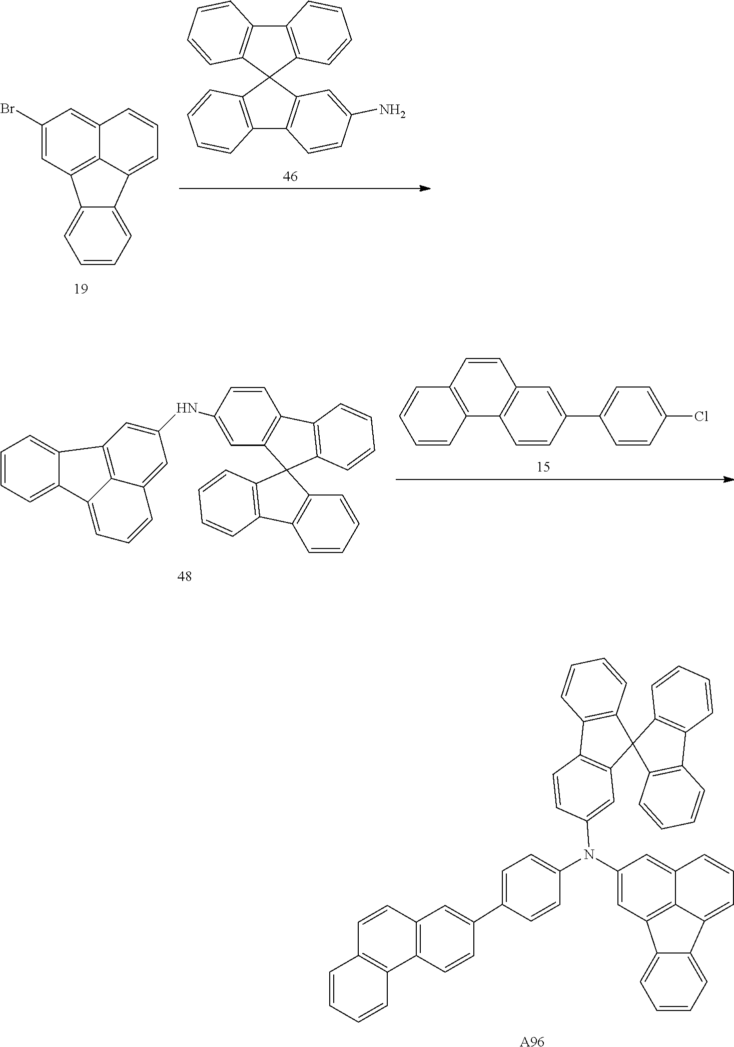

Synthesis of a compound 48: With reference to the synthesis process and post-treatment and purification methods of the compound 20, only the corresponding raw materials were required to be changed. Mass spectrometry was as follows: 532.6 (M+H).

Synthesis of a compound A96: With reference to the synthesis process and post-treatment and purification methods of the compound A1, only the corresponding raw materials were required to be changed, and 6.07 g of a white solid compound A96 with a yield of 65.3% was obtained. 6.07 g of the crude product A96 was sublimated and purified to obtain 4.02 g of a sublimated product A96 with a yield of 66.2%. Mass spectrometry was as follows: 785.0 (M+H). ¹H NMR (400 MHz, CDCl₃) δ 9.11 (d, 1H), 8.70 (dd, 1H), 8.51-8.35 (m, 4H), 8.26 (d, 1H), 8.10 (m, 2H), 8.02-7.83 (m, 6H), 7.78 (dd, J=29.9 Hz, 2H), 7.64 (m, J=17.5 Hz, 3H), 7.55 (dd, J=5.0 Hz, 3H), 7.44 (dd, 1H), 7.36 (m, J=13.6 Hz, 3H), 7.25 (m, J=11.8 Hz, 6H), 6.95 (m, 2H), 6.44 (m, J=17.7 Hz, 2H).

### Example 20: Synthesis of a Compound A99

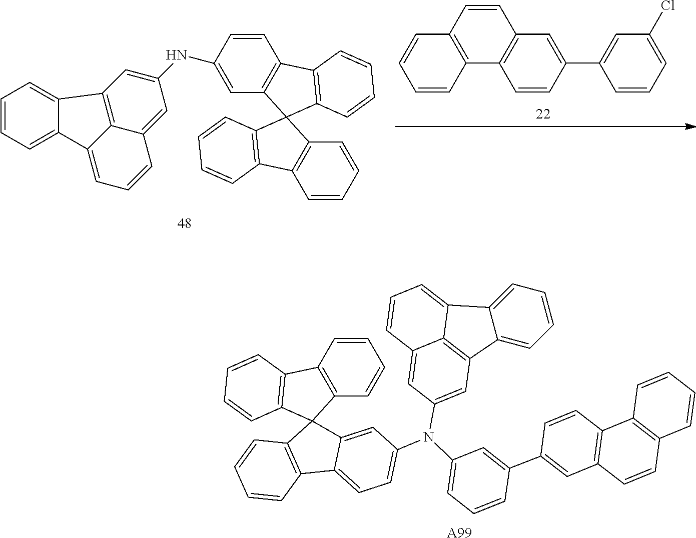

Synthesis of a compound A99: With reference to the synthesis process and post-treatment and purification methods of the compound A1, only the corresponding raw materials were required to be changed, and 6.74 g of a white solid compound A99 with a yield of 67.2% was obtained. 6.74 g of the crude product A99 was sublimated and purified to obtain 4.87 g of a sublimated product A99 with a yield of 72.2%. Mass spectrometry was as follows: 785.0 (M+H). ¹H NMR (400 MHz, CDCl₃) δ 9.11 (d, 1H), δ 8.70 (dd, 1H), 8.48-8.37 (m, 4H), 8.34 (d, 1H), 8.10 (m, 2H), 7.96-7.79 (m, 9H), 7.76 (m, J=13.7 Hz, 2H), 7.71 (m, J=35.0 Hz, 1H), 7.62 (m, J=10.0 Hz, 2H), 7.55 (m, J=5.0 Hz, 2H), 7.34 (dd, 1H), 7.23 (dt, J=31.4, 5.0 Hz, 9H), 6.95 (d, 1H), 6.26 (dd, 1H).

### Example 21: Synthesis of a Compound A101

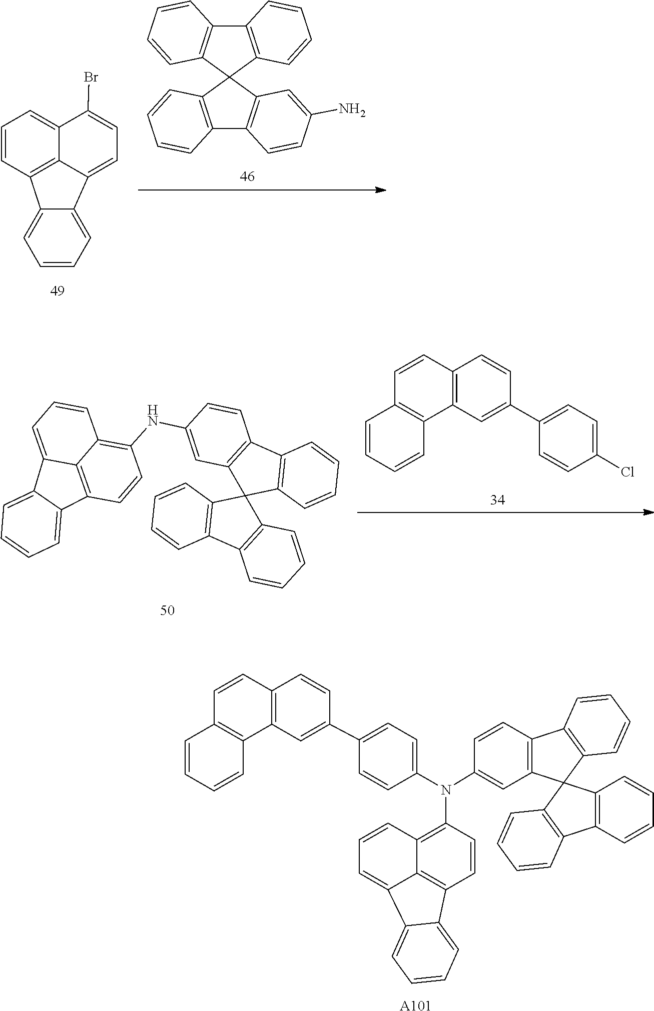

Synthesis of a compound 50: With reference to the synthesis process and post-treatment and purification methods of the compound 20, only the corresponding raw materials were required to be changed. Mass spectrometry was as follows: 532.6 (M+H).

Synthesis of a compound A101: With reference to the synthesis process and post-treatment and purification methods of the compound A1, only the corresponding raw materials were required to be changed, and 8.69 g of a white solid compound A101 with a yield of 67.7% was obtained. 8.69 g of the crude product A101 was sublimated and purified to obtain 5.88 g of a sublimated product A101 with a yield of 67.6%. Mass spectrometry was as follows: 785.0 (M+H). ¹H NMR (400 MHz, CDCl₃) δ 9.01 (d, 1H), 8.85 (dd, 1H), 8.50-8.31 (m, 4H), 8.10 (m, 2H), 7.97-7.83 (m, 5H), 7.84-7.73 (m, 6H), 7.65 (d, J=25.0 Hz, 2H), 7.55 (m, 3H), 7.37 (m, J=10.8 Hz, 4H), 7.24 (m, J=5.0 Hz, 5H), 7.01 (m, 2H), 6.50 (dd, 1H), 6.38 (d, 1H).

### Example 22: Synthesis of a Compound A108

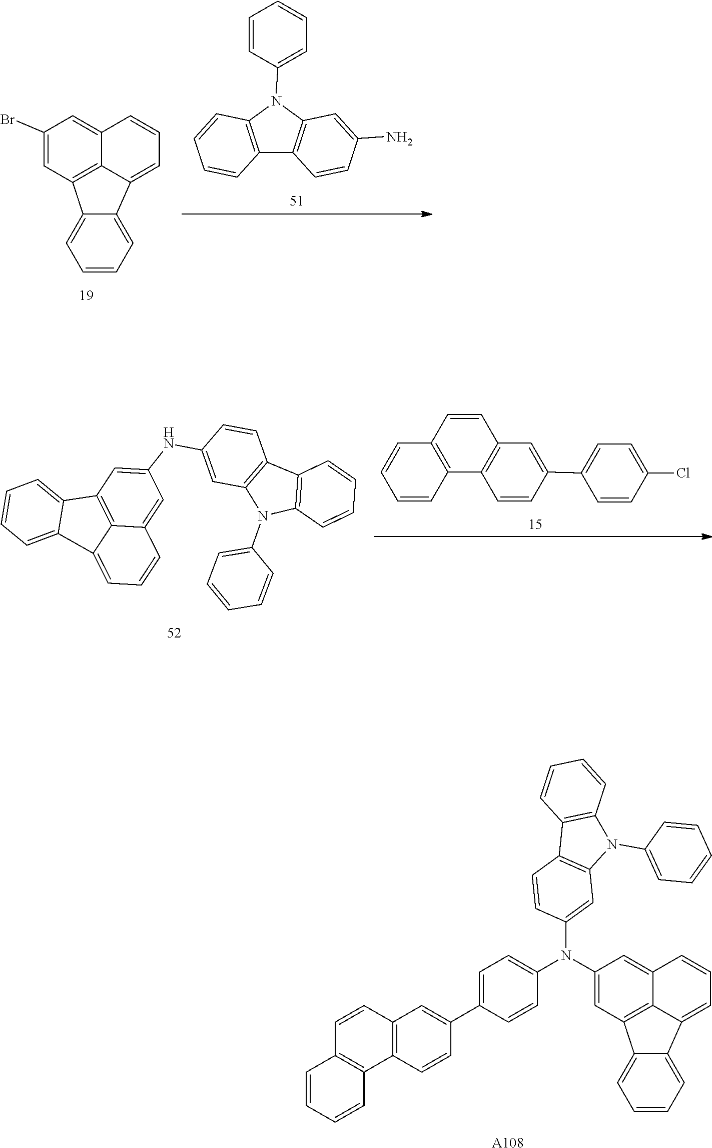

Synthesis of a compound 52: With reference to the synthesis process and post-treatment and purification methods of the compound 20, only the corresponding raw materials were required to be changed. Mass spectrometry was as follows: 459.6 (M+H).

Synthesis of a compound A108: With reference to the synthesis process and post-treatment and purification methods of the compound A1, only the corresponding raw materials were required to be changed, and 7.61 g of a white solid compound A108 with a yield of 59.7% was obtained. 7.61 g of the crude product A108 was sublimated and purified to obtain 4.87 g of a sublimated product A108 with a yield of 63.9%. Mass spectrometry was as follows: 711.8 (M+H). ¹H NMR (400 MHz, CDCl₃) δ 9.11 (d, 1H), 8.70 (dd, 1H), 8.55 (dd, 1H), 8.50 (d, 1H), 8.43 (t, J=2.5 Hz, 4H), 8.24 (d, 1H), 8.10 (dd, 2H), 7.91 (m, J=10.0 Hz, 2H), 7.75 (dd, J=2.3 Hz, 2H), 7.72-7.45 (m, 12H), 7.37 (d, 2H), 7.33-7.21 (m, 3H), 7.13 (m, J=25.0 Hz, 2H).

### Application Example: Manufacture of an Organic Electroluminescent Device

A glass substrate with a size of 50 mm*50 mm*1.0 mm including an ITO (100 nm) transparent electrode was ultrasonically cleaned in ethanol for 10 minutes, dried at 150° C., and then treated with N2 plasma for 30 minutes. The washed glass substrate was installed on a substrate support of a vacuum evaporation device. At first, a compound HATCN for covering the transparent electrode was evaporated on the surface of the side having a transparent electrode line to form a thin film with a thickness of 5 nm. Next, a layer of HTM1 was evaporated to form a thin film as a hole transport layer 1 (HTL1) with a thickness of 60 nm. Then, a layer of HTM2 was evaporated on the HTM1 thin film to form a thin film as a hole transport layer 2 (HTL2) with a thickness of 10 nm. After that, a main material and a doping material (with a doping proportion of 2%) were co-evaporated on the HTM2 film layer to obtain a film with a thickness of 25 nm, where a ratio of the main material to the doping material was 90%:10%. An electron transport layer (ETL, 30 nm) was evaporated on a light-emitting layer in sequence to serve as an electron transport material according to combinations in the following table. LiQ (1 nm) was evaporated on the electron transport material layer to serve as an electron injection material. At last, a mixture of Mg and Ag (100 nm, at a ratio of 1:9) was co-evaporated to serve as a cathode material.

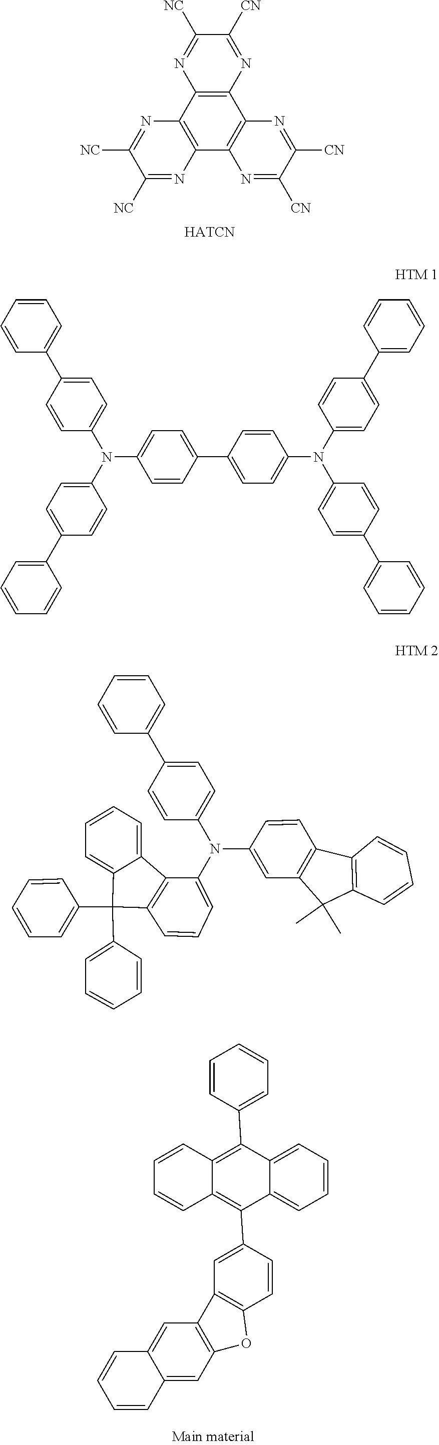

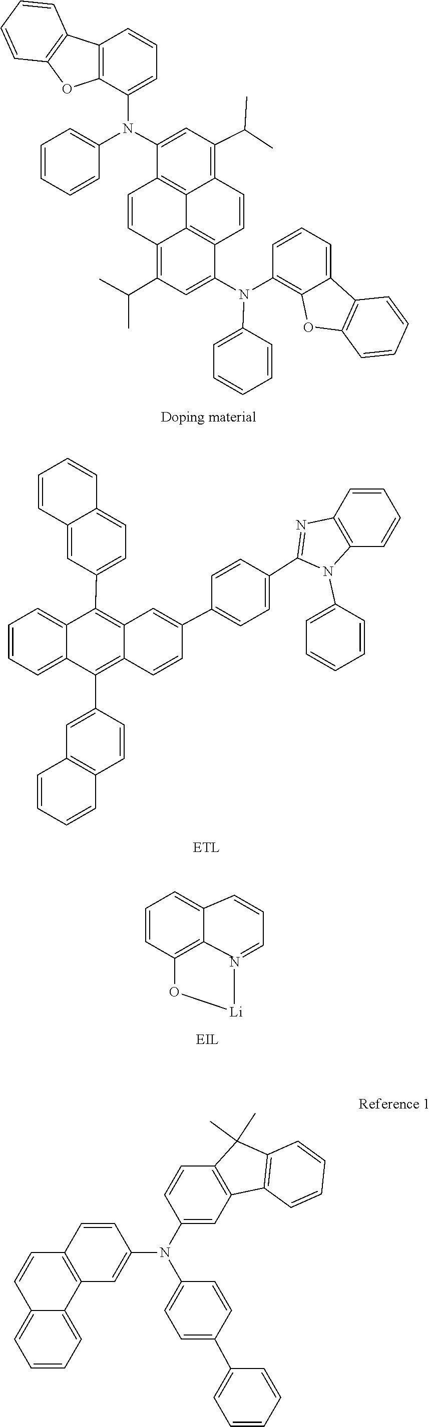

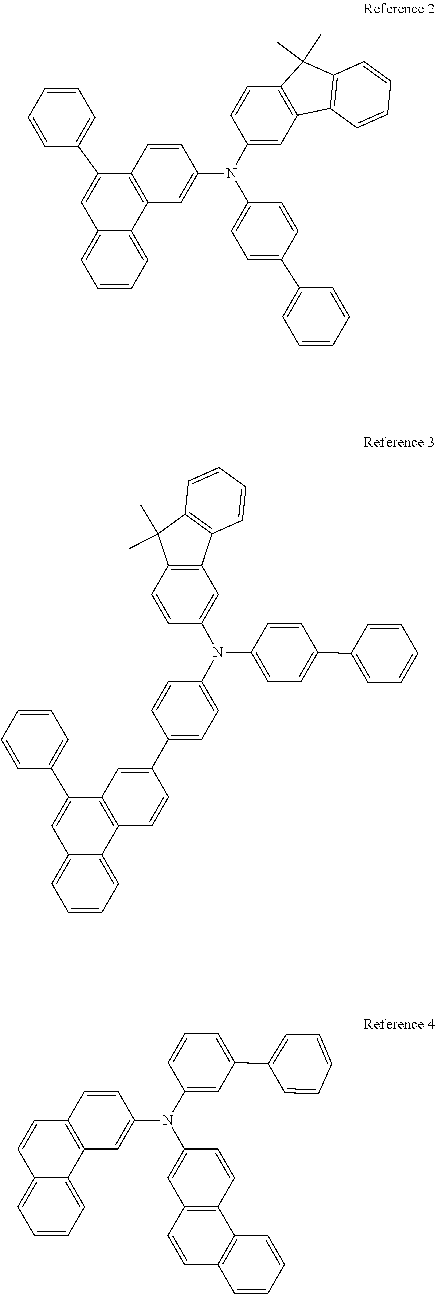 [SMILES: CC1(C)c2ccccc2-c2cc(N(c3ccc(-c4ccccc4)cc3)c3ccc4c(-c5ccccc5)cc5ccccc5c4c3)ccc21 ; CC1(C)c2ccccc2-c2cc(N(c3ccc(-c4ccccc4)cc3)c3ccc(-c4ccc5c(c4)c(-c4ccccc4)cc4ccccc45)cc3)ccc21 ; c1ccc(-c2cccc(N(c3ccc4c(ccc5ccccc54)c3)c3ccc4ccc5ccccc5c4c3)c2)cc1]

### Evaluation

Properties of a device obtained above were tested. In various examples and comparative examples, a constant-current power supply (Keithley 2400) was used, a current at a fixed density was used for flowing through light-emitting elements, and a spectroradiometer (CS 2000) was used for testing the light-emitting spectrum. Meanwhile, the voltage value was measured, and the time (LT90) when the brightness was reduced to 90% of an initial brightness was tested. Results are shown in the following Table 1.

TABLE 1

| | | | Starting | External | |
|---|---|---|---|---|---|
| | | | voltage V | quantum | |
| | | | @ 1000 | efficiency | LT90 @ |
| | HTL1 | HTL2 | nits | (%) | 1000 nits |
| Example 1 | HTM1 | Compound A1 | 3.79 | 9.39 | 72 |
| Example 2 | HTM1 | Compound A2 | 3.78 | 9.76 | 81 |
| Example 3 | HTM1 | Compound A4 | 3.83 | 9.64 | 83 |
| Example 4 | HTM1 | Compound A21 | 3.82 | 9.55 | 88 |
| Example 5 | HTM1 | Compound A24 | 3.83 | 9.75 | 94 |
| Example 6 | HTM1 | Compound A27 | 3.86 | 10.12 | 99 |
| Example 7 | HTM1 | Compound A33 | 3.81 | 10.03 | 102 |
| Example 8 | HTM1 | Compound A70 | 3.62 | 9.81 | 92 |
| Example 9 | HTM1 | Compound A72 | 3.69 | 9.67 | 106 |
| Example 10 | HTM1 | Compound A81 | 3.67 | 9.94 | 121 |
| Example 11 | Compound A94 | HTM2 | 3.67 | 9.72 | 85 |
| Example 12 | Compound A96 | HTM2 | 3.69 | 10.33 | 118 |
| Example 13 | Compound A99 | HTM2 | 3.62 | 9.86 | 98 |
| Example 14 | Compound A101 | HTM2 | 3.73 | 9.67 | 84 |
| Example 15 | HTM1 | Compound A108 | 3.82 | 10.28 | 99 |
| Example 16 | HTM1 | Compound A118 | 3.67 | 9.98 | 109 |
| Example 17 | HTM1 | Compound A138 | 3.62 | 10.19 | 121 |
| Example 18 | HTM1 | Compound A150 | 3.64 | 10.43 | 133 |
| Example 19 | HTM1 | Compound A158 | 3.74 | 9.95 | 102 |
| Example 20 | HTM1 | Compound A159 | 3.79 | 10.18 | 112 |
| Example 21 | HTM1 | Compound A174 | 3.70 | 10.17 | 116 |
| Example 22 | HTM1 | Compound A181 | 3.76 | 10.86 | 127 |
| Comparative | HTM1 | HTM2 | 3.97 | 8.45 | 35 |
| Example 1 | | | | | |
| Comparative | HTM1 | Reference 1 | 3.92 | 8.83 | 29 |
| Example 2 | | | | | |
| Comparative | HTM1 | Reference 2 | 3.95 | 8.98 | 34 |
| Example 3 | | | | | |
| Comparative | HTM1 | Reference 3 | 3.89 | 9.06 | 46 |
| Example 4 | | | | | |
| Comparative | HTM1 | Reference 4 | 3.88 | 9.23 | 59 |
| Example 5 | | | | | |
| Example 23 | Compound A174 | HTM2 | 3.68 | 10.32 | 122 |
| Example 24 | Compound A174 | Compound A150 | 3.66 | 10.63 | 144 |

Through comparison of the data in the above table, it can be seen that compared with reference compounds, the compound of the present invention used as a hole transport layer or an electron blocking layer in an organic electroluminescent device has the advantages that more excellent properties, such as driving voltage, luminescence efficiency, and device service life, are achieved.

According to the above results, it is indicated that the compound of the present invention has the advantages of great optical, electrical, and thermal stability, high luminescence efficiency, low electric voltage, and long service life, and can be used in organic electroluminescent devices. In particular, the compound has the potential for application in the AMOLED industry as a hole transport layer material or an electron blocking layer material.

1. A compound, having a structural formula as shown in the following formula (1): 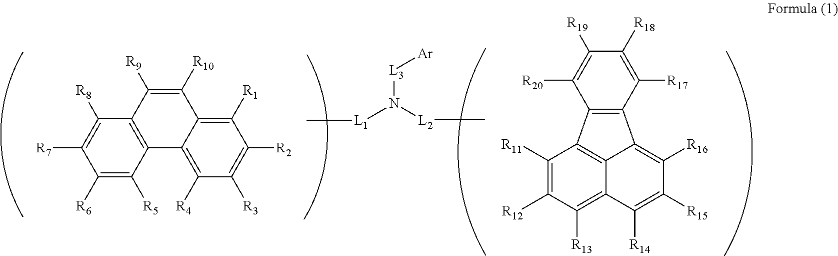
wherein any one of R₁-R₁₀ is a single bond for being bonded to L₁, and the other groups are substituents independently;
any one of R₁₁-R₂₀ is a single bond for being bonded to L₂, and the other groups are substituents independently;
each of the substituents is independently selected from hydrogen, deuterium, halogen, C₁-C₁₀ alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro; or two adjacent substituents are connected into a ring;
each of L₁-L₃ independently refers to a single bond, arylene with a ring forming carbon number of C₆₋₅₀ unsubstituted or substituted with R, and heteroarylene with a ring forming atom number of C₅₋₅₀ unsubstituted or substituted with R;
Ar refers to aryl with a ring forming carbon number of 6-50 unsubstituted or substituted with R, heteroaryl with a ring forming atom number of 5-50 unsubstituted or substituted with R, and a monocyclic or polycyclic C₃-C₆₀ alicyclic ring or aromatic ring unsubstituted or substituted with R; or one or more of carbon atoms in the monocyclic or polycyclic C₃-C₆₀ alicyclic ring or aromatic ring unsubstituted or substituted with R are substituted with at least one heteroatom selected from O, S, N, Se, Si, and Ge;
a heteroatom in the heteroaryl or heteroalkyl is at least one heteroatom selected from O, S, N, Se, Si, and Ge;
and the R is independently selected from deuterium, F, Cl, Br, C₁-C₄ alkyl, C₁-C₄alkoxy, C₃-C₂₀cycloalkyl, C₆-C₁₀ aryl, aralkyl with a carbon number of 7-30 of aryl with a ring forming carbon number of 6-10, alkoxy with a carbon number of 1-20, aryloxy with a ring forming carbon number of 6-10, and at least one group of monosubstituted, disubstituted or trisubstituted silyl, cyano and nitro with substituents selected from alkyl with a carbon number of 1-10 and aryl with a ring forming carbon number of 6-10.

2. The compound according to claim 1, having one of the following structural formulas:

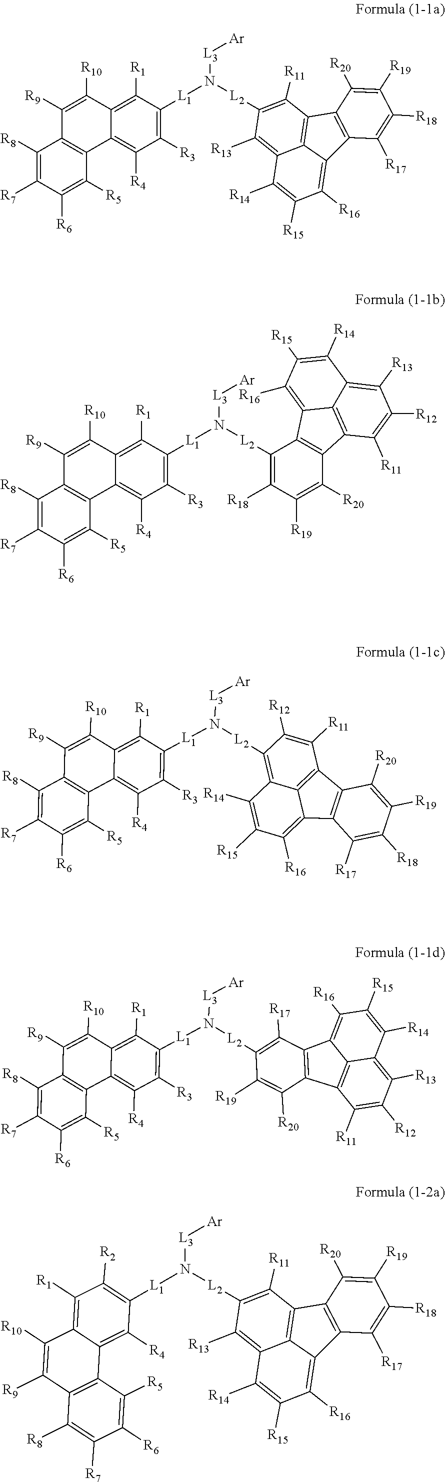

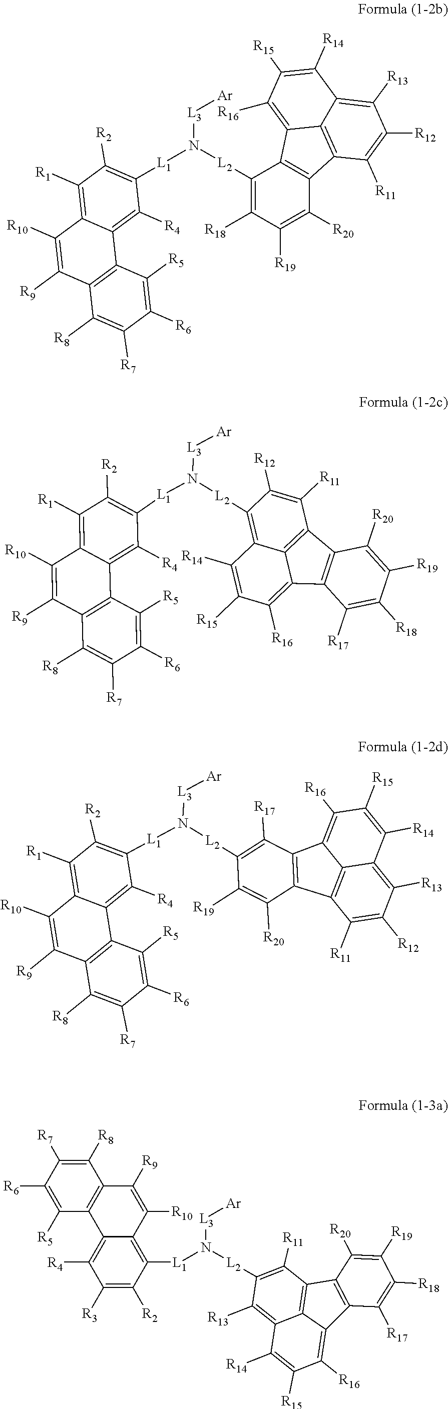

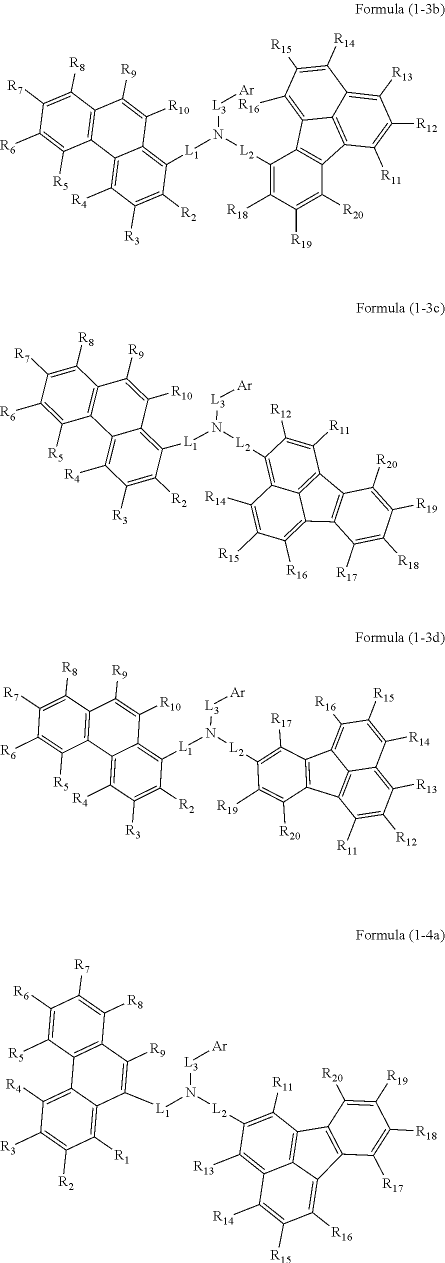

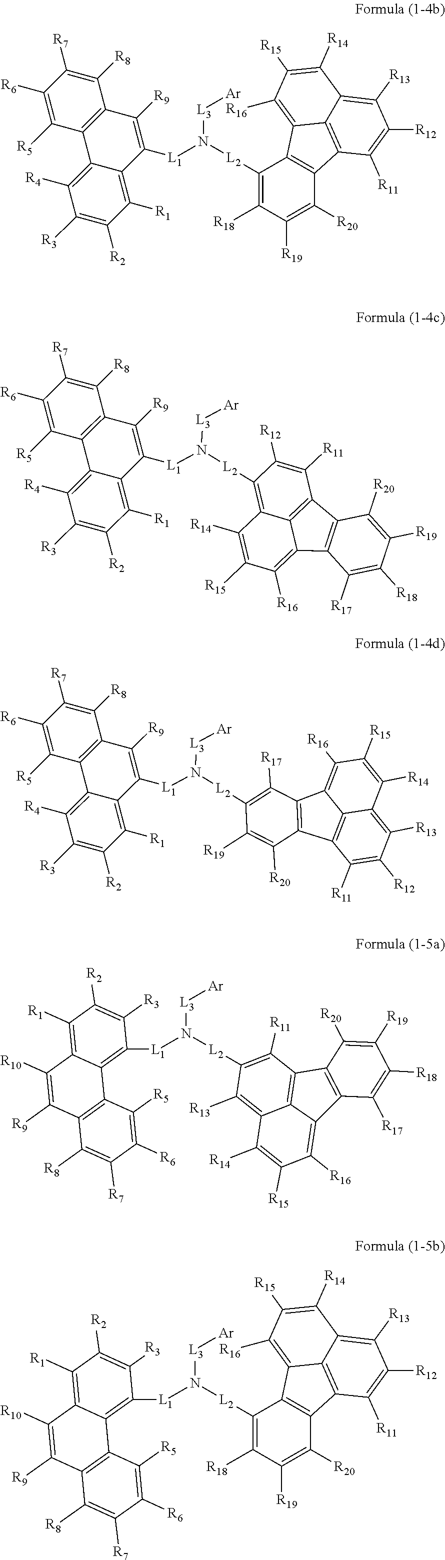

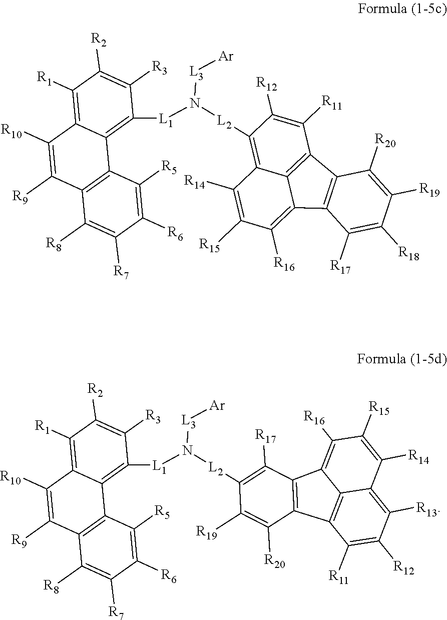

3. The compound according to claim 2, wherein each of the substituents, namely the R₁-R₂₀, is independently selected from hydrogen, deuterium, halogen, C₁-C₄ alkyl, C₃-C₆cycloalkyl, C₆-C₁₄aralkyl, C₁-C₁₄ alkoxy, C₆-C₁₄aryloxy, amino, C₆-C₁₄ aryl, cyano, and nitro;
each of the L₁-L₃ independently refers to a single bond, arylene with a ring forming carbon number of 6-14 unsubstituted or substituted with R, and heteroarylene with a ring forming atom number of 5-13 unsubstituted or substituted with R;
the heteroatom in the heteroaryl is at least one heteroatom selected from O, S, N, and Si;
and the R is independently selected from deuterium, F, Cl, Br, and C₁-C₄ alkyl.

4. The compound according to claim 3, wherein each of the R₁-R₄ and R₉-R₂₀ is independently selected from hydrogen; among the R₅-R₈, three groups are hydrogen, and the other group is hydrogen, C₁-C₄ alkyl, phenyl substituted with C₁-C₄ alkyl, phenyl, or naphthyl;
and each of the L₁-L₃ independently refers to a single bond, phenylene unsubstituted or substituted with C₁-C₄ alkyl, and naphthylene unsubstituted or substituted with C₁-C₄ alkyl.

5. The compound according to claim 2, wherein among the R₁-R₂₀, two adjacent substituents may be connected into a ring with a ring fused structure as shown in the following formula (2) or (3): 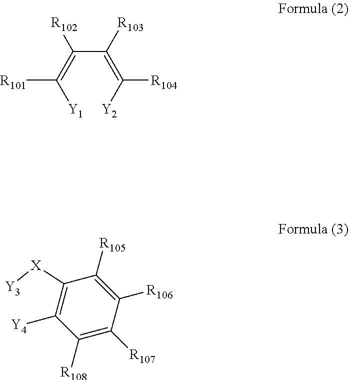
wherein Y₁, Y₂, Y₃, and Y₄ refer to positions connected to a ring; X is independently selected from O, S, SO₂, NR₁₀₉, CR₁₁₀R₁₁₁, and Si R₁₁₂R₁₁₃; and each of R₁₀₁-R₁₀₉ is independently selected from hydrogen, deuterium, halogen, C₁-C₁₀ alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro.

6. The compound according to any one of claims 1 to 5, wherein the Ar is as shown in any one of the following formulas (a) to (x): 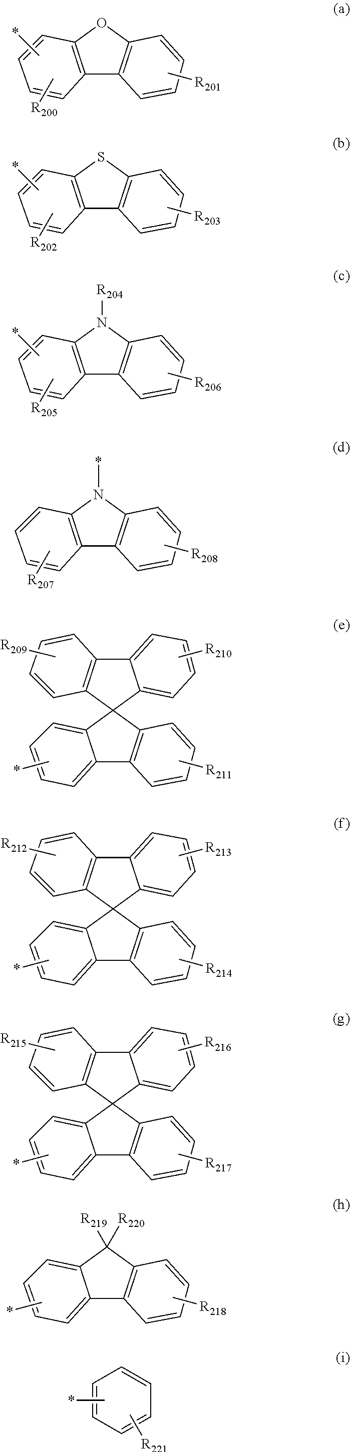 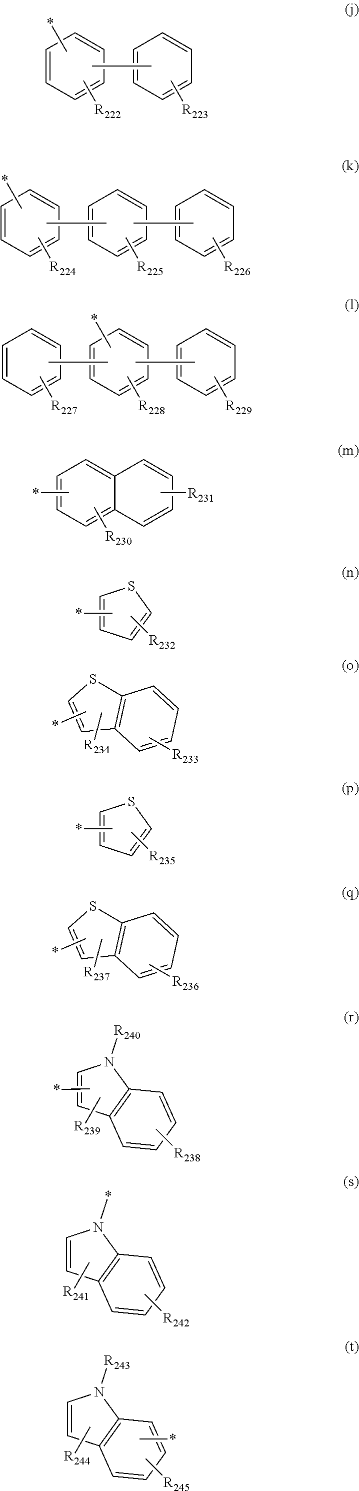 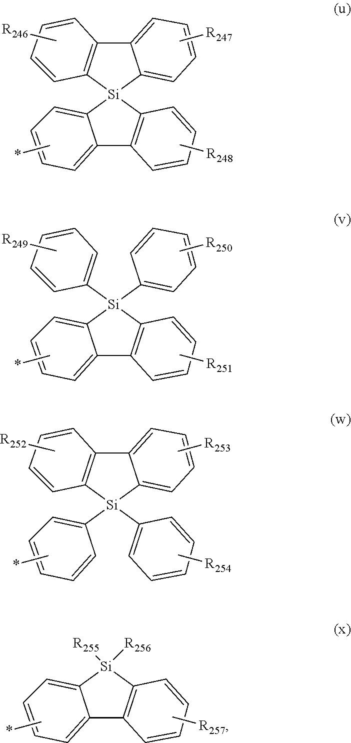
wherein each of R₂₀₀-R₂₅₇ independently refers to no substitution to a maximum possible substituent number; when the R₂₀₀-R₂₅₇ are substituents, each of the R₂₀₀-R₂₅₇ is independently selected from deuterium, halogen, C₁-C₁₀ alkyl unsubstituted or substituted with R, C₃-C₂₀cycloalkyl unsubstituted or substituted with R, C₁-C₁₀heteroalkyl unsubstituted or substituted with R, C₆-C₃₀aralkyl unsubstituted or substituted with R, C₁-C₁₀alkoxy unsubstituted or substituted with R, C₆-C₃₀aryloxy unsubstituted or substituted with R, amino, C₃-C₃₀ silyl unsubstituted or substituted with R, C₆-C₃₀ aryl unsubstituted or substituted with R, C₃-C₃₀heteroaryl unsubstituted or substituted with R, cyano, and nitro; or two adjacent groups are connected into a ring;
and * refers to a bonding position connected to the L₃ in the formula (1).

7. The compound according to claim 6, wherein each of the R₂₀₀-R₂₅₇ is independently selected from hydrogen, C₁-C₄ alkyl, phenyl unsubstituted or substituted with C₁-C₄ alkyl, and naphthyl unsubstituted or substituted with C₁-C₄ alkyl.

8. The compound according to claim 2, having one of the following structural formulas:

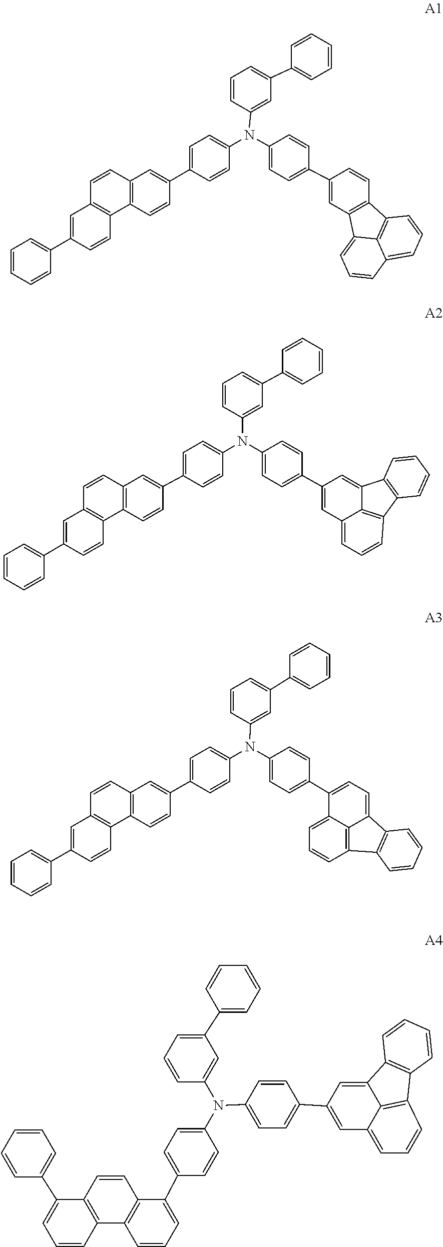 [SMILES: c1ccc(-c2cccc(N(c3ccc(-c4ccc5c(c4)-c4cccc6cccc-5c46)cc3)c3ccc(-c4ccc5c(ccc6cc(-c7ccccc7)ccc65)c4)cc3)c2)cc1 ; c1ccc(-c2cccc(N(c3ccc(-c4cc5c6c(cccc6c4)-c4ccccc4-5)cc3)c3ccc(-c4ccc5c(ccc6cc(-c7ccccc7)ccc65)c4)cc3)c2)cc1 ; c1ccc(-c2cccc(N(c3ccc(-c4ccc5c(ccc6cc(-c7ccccc7)ccc65)c4)cc3)c3ccc(-c4ccc5c6c(cccc46)-c4ccccc4-5)cc3)c2)cc1 ; c1ccc(-c2cccc(N(c3ccc(-c4cc5c6c(cccc6c4)-c4ccccc4-5)cc3)c3ccc(-c4cccc5c4ccc4c(-c6ccccc6)cccc45)cc3)c2)cc1]

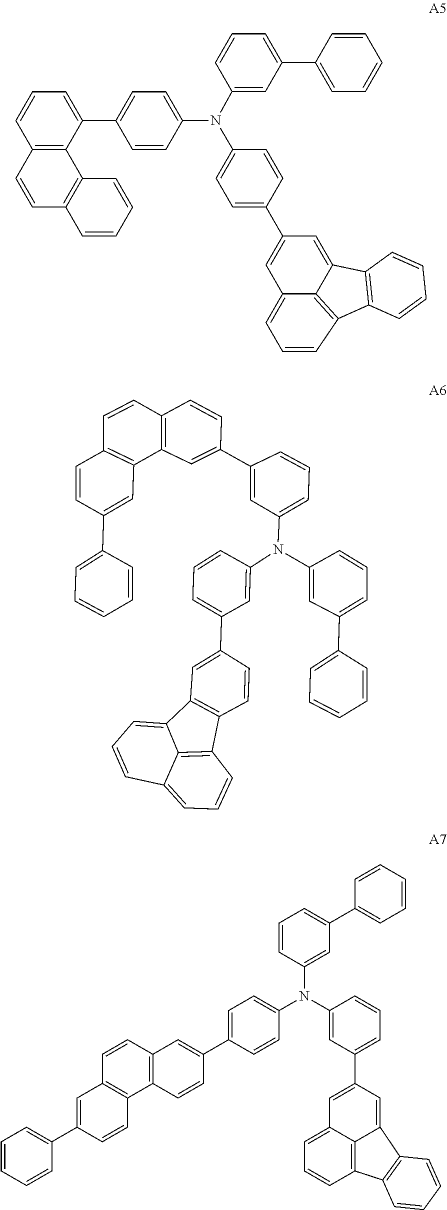 [SMILES: c1ccc(-c2cccc(N(c3ccc(-c4cc5c6c(cccc6c4)-c4ccccc4-5)cc3)c3ccc(-c4cccc5ccc6ccccc6c45)cc3)c2)cc1 ; c1ccc(-c2cccc(N(c3cccc(-c4ccc5c(c4)-c4cccc6cccc-5c46)c3)c3cccc(-c4ccc5ccc6ccc(-c7ccccc7)cc6c5c4)c3)c2)cc1 ; c1ccc(-c2cccc(N(c3ccc(-c4ccc5c(ccc6cc(-c7ccccc7)ccc65)c4)cc3)c3cccc(-c4cc5c6c(cccc6c4)-c4ccccc4-5)c3)c2)cc1]

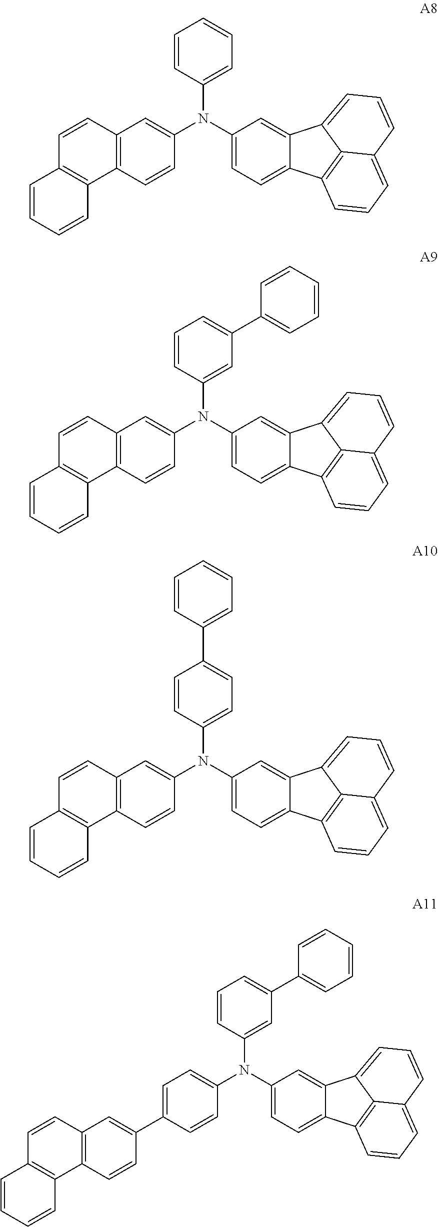 [SMILES: c1ccc(N(c2ccc3c(c2)-c2cccc4cccc-3c24)c2ccc3c(ccc4ccccc43)c2)cc1 ; c1ccc(-c2cccc(N(c3ccc4c(c3)-c3cccc5cccc-4c35)c3ccc4c(ccc5ccccc54)c3)c2)cc1 ; c1ccc(-c2ccc(N(c3ccc4c(c3)-c3cccc5cccc-4c35)c3ccc4c(ccc5ccccc54)c3)cc2)cc1 ; c1ccc(-c2cccc(N(c3ccc(-c4ccc5c(ccc6ccccc65)c4)cc3)c3ccc4c(c3)-c3cccc5cccc-4c35)c2)cc1]

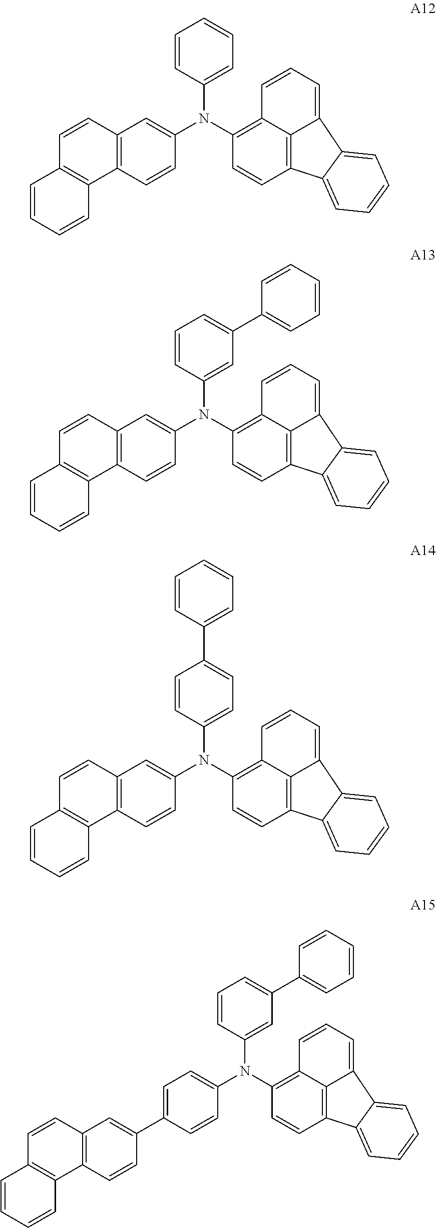 [SMILES: c1ccc(N(c2ccc3c(ccc4ccccc43)c2)c2ccc3c4c(cccc24)-c2ccccc2-3)cc1 ; c1ccc(-c2cccc(N(c3ccc4c(ccc5ccccc54)c3)c3ccc4c5c(cccc35)-c3ccccc3-4)c2)cc1 ; c1ccc(-c2ccc(N(c3ccc4c(ccc5ccccc54)c3)c3ccc4c5c(cccc35)-c3ccccc3-4)cc2)cc1 ; c1ccc(-c2cccc(N(c3ccc(-c4ccc5c(ccc6ccccc65)c4)cc3)c3ccc4c5c(cccc35)-c3ccccc3-4)c2)cc1]

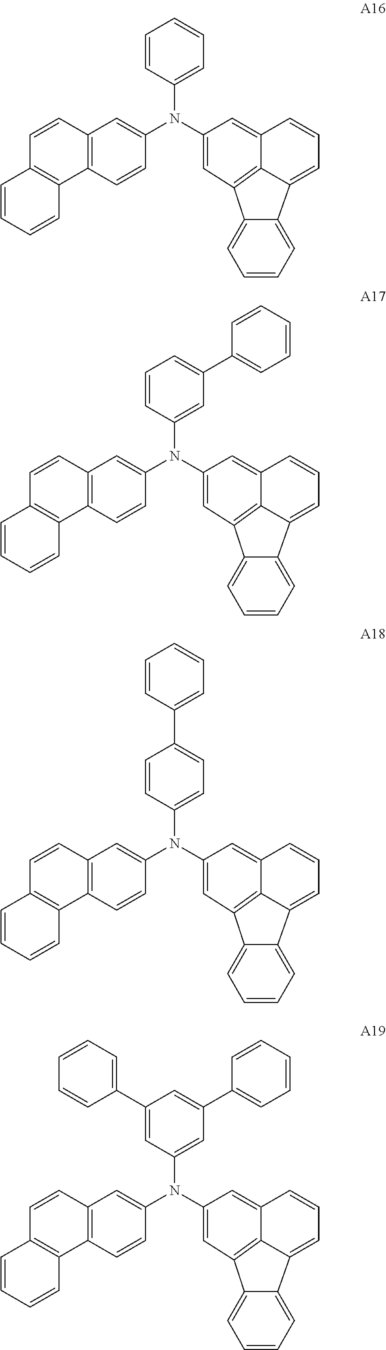

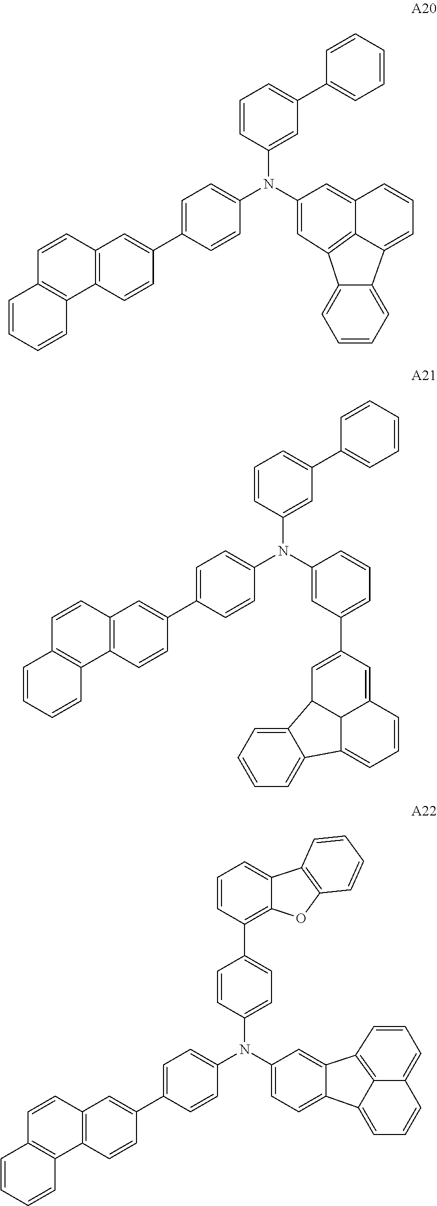 [SMILES: c1ccc(-c2cccc(N(c3ccc(-c4ccc5c(ccc6ccccc65)c4)cc3)c3cc4c5c(cccc5c3)-c3ccccc3-4)c2)cc1 ; C1=CC2=CC(c3cccc(N(c4ccc(-c5ccc6c(ccc7ccccc76)c5)cc4)c4cccc(-c5ccccc5)c4)c3)=CC3c4ccccc4C(=C1)C23 ; c1cc2c3c(cccc3c1)-c1cc(N(c3ccc(-c4ccc5c(ccc6ccccc65)c4)cc3)c3ccc(-c4cccc5c4oc4ccccc45)cc3)ccc1-2]

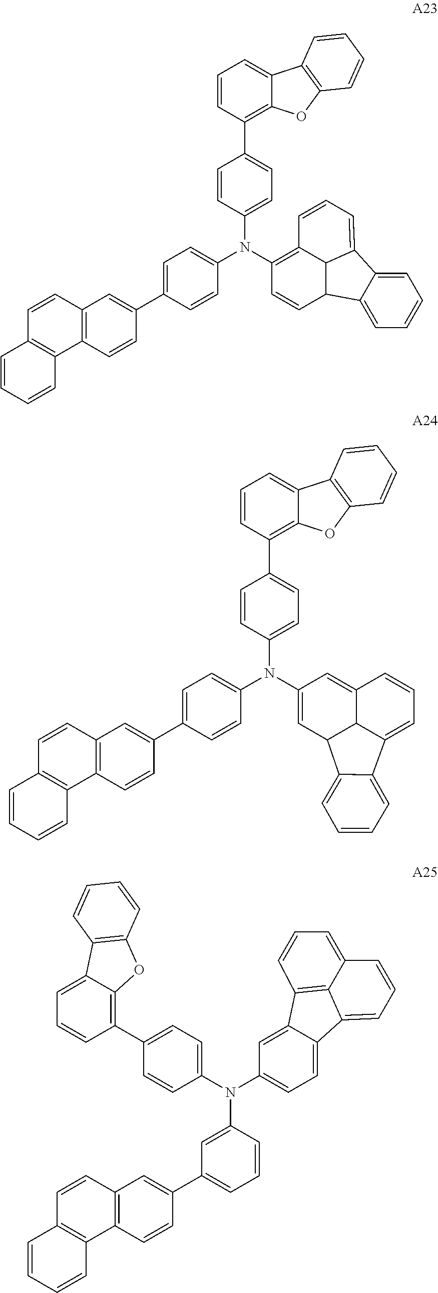 [SMILES: C1=CC2=C(N(c3ccc(-c4ccc5c(ccc6ccccc65)c4)cc3)c3ccc(-c4cccc5c4oc4ccccc45)cc3)C=CC3c4ccccc4C(=C1)C23 ; C1=CC2=CC(N(c3ccc(-c4ccc5c(ccc6ccccc65)c4)cc3)c3ccc(-c4cccc5c4oc4ccccc45)cc3)=CC3c4ccccc4C(=C1)C23 ; c1cc(-c2ccc3c(ccc4ccccc43)c2)cc(N(c2ccc(-c3cccc4c3oc3ccccc34)cc2)c2ccc3c(c2)-c2cccc4cccc-3c24)c1]

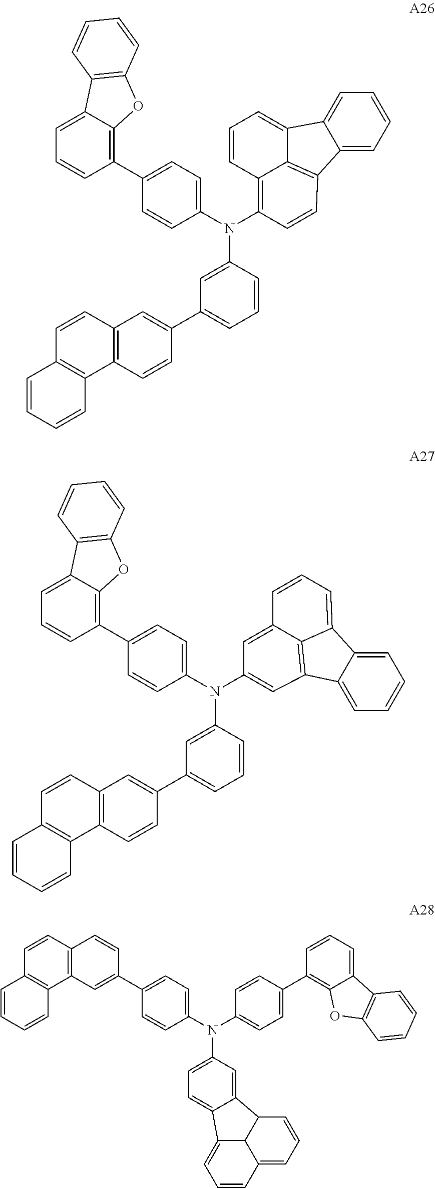

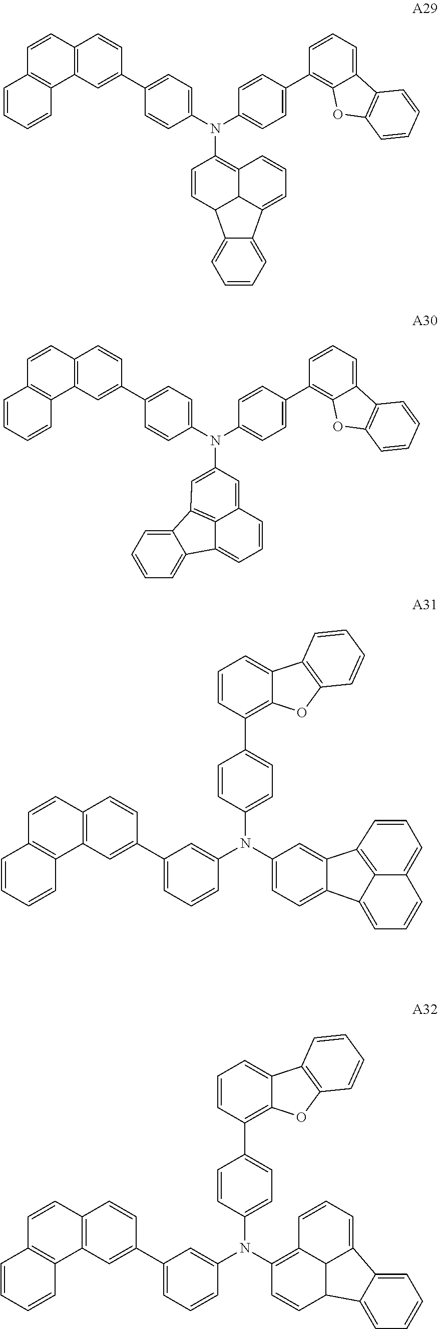 [SMILES: C1=CC2=C(N(c3ccc(-c4ccc5ccc6ccccc6c5c4)cc3)c3ccc(-c4cccc5c4oc4ccccc45)cc3)C=CC3c4ccccc4C(=C1)C23 ; c1ccc2c(c1)-c1cccc3cc(N(c4ccc(-c5ccc6ccc7ccccc7c6c5)cc4)c4ccc(-c5cccc6c5oc5ccccc56)cc4)cc-2c13 ; c1cc(-c2ccc3ccc4ccccc4c3c2)cc(N(c2ccc(-c3cccc4c3oc3ccccc34)cc2)c2ccc3c(c2)-c2cccc4cccc-3c24)c1 ; C1=CC2=C(N(c3ccc(-c4cccc5c4oc4ccccc45)cc3)c3cccc(-c4ccc5ccc6ccccc6c5c4)c3)C=CC3c4ccccc4C(=C1)C23]

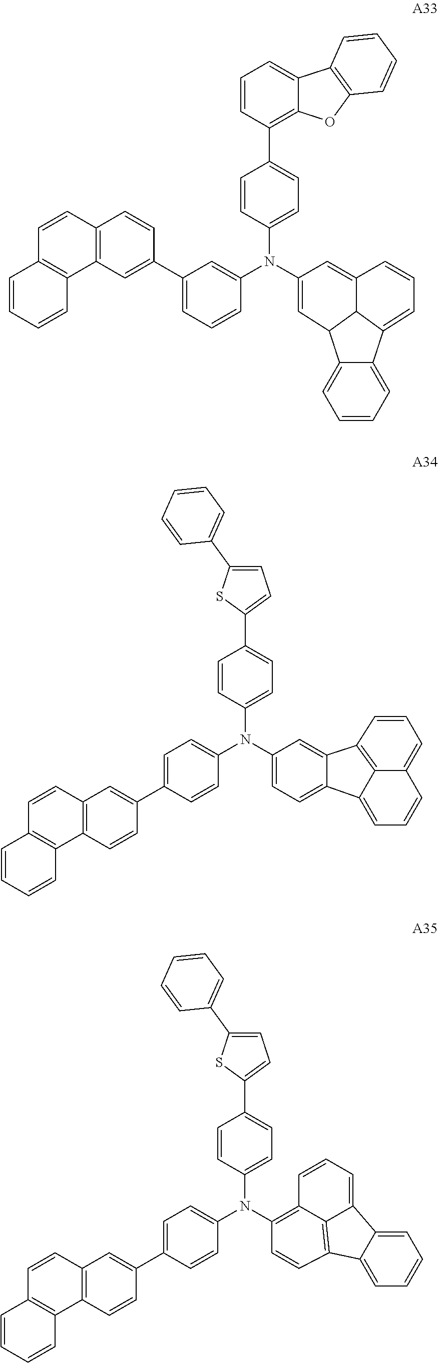 [SMILES: C1=CC2=CC(N(c3ccc(-c4cccc5c4oc4ccccc45)cc3)c3cccc(-c4ccc5ccc6ccccc6c5c4)c3)=CC3c4ccccc4C(=C1)C23 ; c1ccc(-c2ccc(-c3ccc(N(c4ccc(-c5ccc6c(ccc7ccccc76)c5)cc4)c4ccc5c(c4)-c4cccc6cccc-5c46)cc3)s2)cc1 ; c1ccc(-c2ccc(-c3ccc(N(c4ccc(-c5ccc6c(ccc7ccccc76)c5)cc4)c4ccc5c6c(cccc46)-c4ccccc4-5)cc3)s2)cc1]

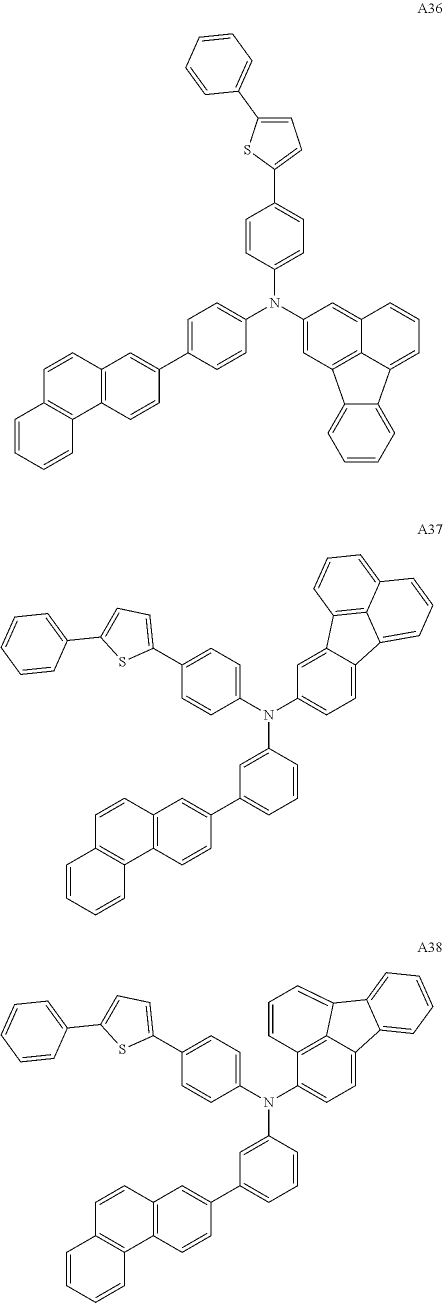 [SMILES: c1ccc(-c2ccc(-c3ccc(N(c4ccc(-c5ccc6c(ccc7ccccc76)c5)cc4)c4cc5c6c(cccc6c4)-c4ccccc4-5)cc3)s2)cc1 ; c1ccc(-c2ccc(-c3ccc(N(c4cccc(-c5ccc6c(ccc7ccccc76)c5)c4)c4ccc5c(c4)-c4cccc6cccc-5c46)cc3)s2)cc1 ; c1ccc(-c2ccc(-c3ccc(N(c4cccc(-c5ccc6c(ccc7ccccc76)c5)c4)c4ccc5c6c(cccc46)-c4ccccc4-5)cc3)s2)cc1]

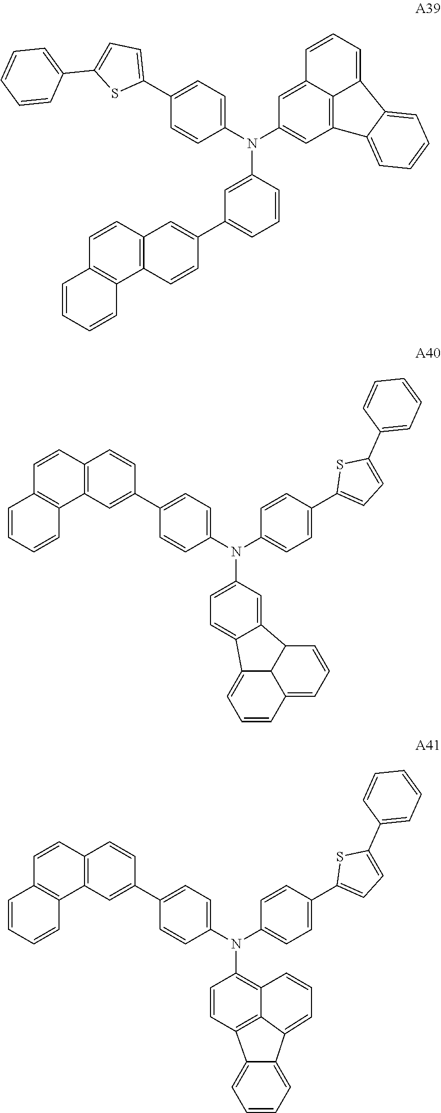 [SMILES: c1ccc(-c2ccc(-c3ccc(N(c4cccc(-c5ccc6c(ccc7ccccc76)c5)c4)c4cc5c6c(cccc6c4)-c4ccccc4-5)cc3)s2)cc1 ; C1=CC2=CC=CC3c4cc(N(c5ccc(-c6ccc7ccc8ccccc8c7c6)cc5)c5ccc(-c6ccc(-c7ccccc7)s6)cc5)ccc4C(=C1)C23 ; c1ccc(-c2ccc(-c3ccc(N(c4ccc(-c5ccc6ccc7ccccc7c6c5)cc4)c4ccc5c6c(cccc46)-c4ccccc4-5)cc3)s2)cc1]

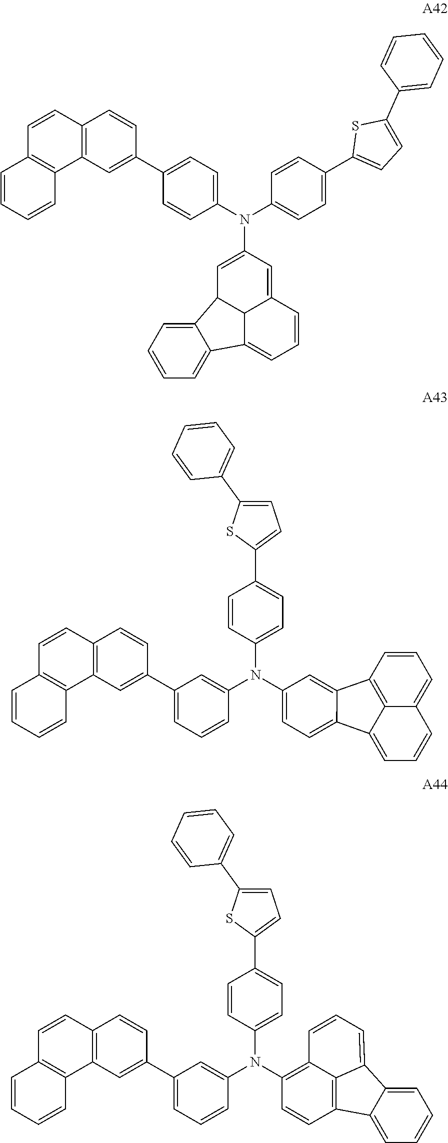 [SMILES: C1=CC2=CC(N(c3ccc(-c4ccc5ccc6ccccc6c5c4)cc3)c3ccc(-c4ccc(-c5ccccc5)s4)cc3)=CC3c4ccccc4C(=C1)C23 ; c1ccc(-c2ccc(-c3ccc(N(c4cccc(-c5ccc6ccc7ccccc7c6c5)c4)c4ccc5c(c4)-c4cccc6cccc-5c46)cc3)s2)cc1 ; c1ccc(-c2ccc(-c3ccc(N(c4cccc(-c5ccc6ccc7ccccc7c6c5)c4)c4ccc5c6c(cccc46)-c4ccccc4-5)cc3)s2)cc1]

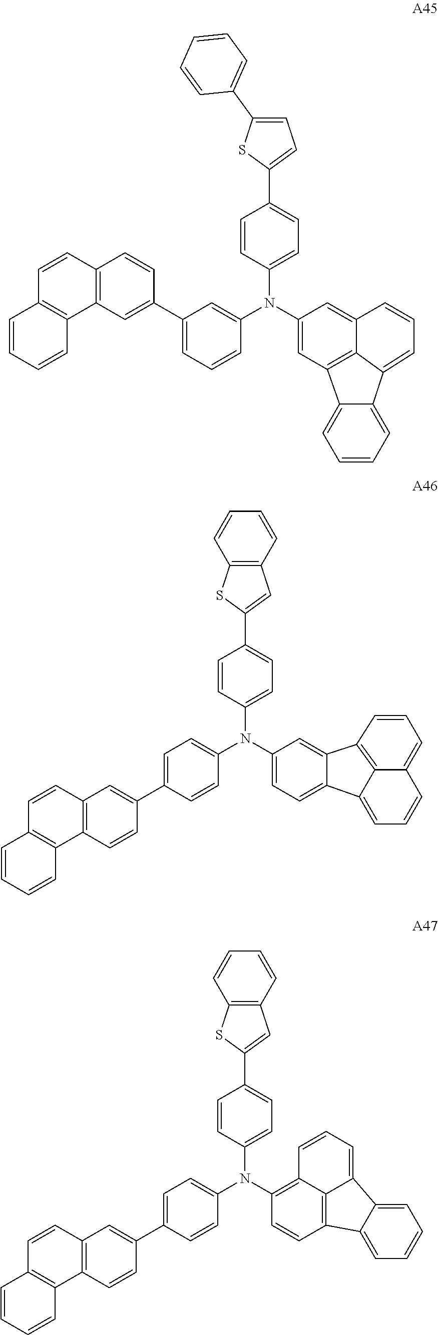 [SMILES: c1ccc(-c2ccc(-c3ccc(N(c4cccc(-c5ccc6ccc7ccccc7c6c5)c4)c4cc5c6c(cccc6c4)-c4ccccc4-5)cc3)s2)cc1 ; c1ccc2sc(-c3ccc(N(c4ccc(-c5ccc6c(ccc7ccccc76)c5)cc4)c4ccc5c(c4)-c4cccc6cccc-5c46)cc3)cc2c1 ; c1ccc2c(c1)-c1cccc3c(N(c4ccc(-c5ccc6c(ccc7ccccc76)c5)cc4)c4ccc(-c5cc6ccccc6s5)cc4)ccc-2c13]

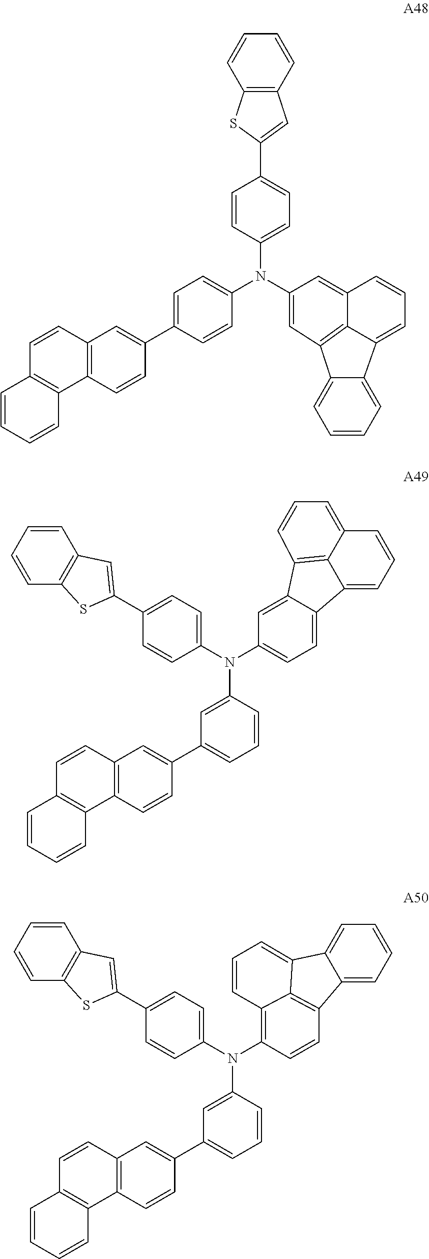 [SMILES: c1ccc2c(c1)-c1cccc3cc(N(c4ccc(-c5ccc6c(ccc7ccccc76)c5)cc4)c4ccc(-c5cc6ccccc6s5)cc4)cc-2c13 ; c1cc(-c2ccc3c(ccc4ccccc43)c2)cc(N(c2ccc(-c3cc4ccccc4s3)cc2)c2ccc3c(c2)-c2cccc4cccc-3c24)c1 ; c1cc(-c2ccc3c(ccc4ccccc43)c2)cc(N(c2ccc(-c3cc4ccccc4s3)cc2)c2ccc3c4c(cccc24)-c2ccccc2-3)c1]

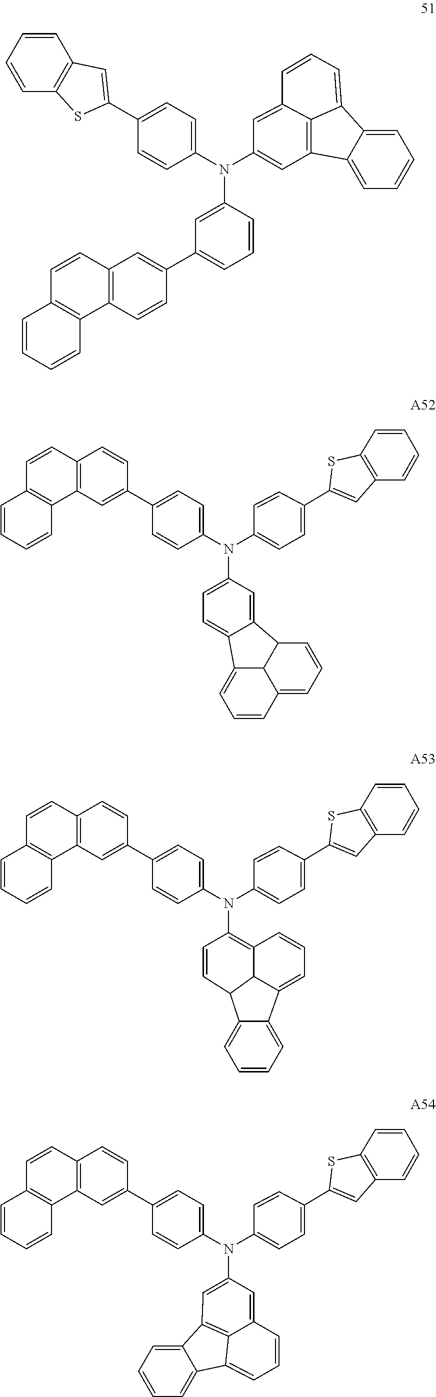 [SMILES: c1cc(-c2ccc3c(ccc4ccccc43)c2)cc(N(c2ccc(-c3cc4ccccc4s3)cc2)c2cc3c4c(cccc4c2)-c2ccccc2-3)c1 ; C1=CC2=CC=CC3c4cc(N(c5ccc(-c6ccc7ccc8ccccc8c7c6)cc5)c5ccc(-c6cc7ccccc7s6)cc5)ccc4C(=C1)C23 ; C1=CC2=C(N(c3ccc(-c4ccc5ccc6ccccc6c5c4)cc3)c3ccc(-c4cc5ccccc5s4)cc3)C=CC3c4ccccc4C(=C1)C23 ; c1ccc2c(c1)-c1cccc3cc(N(c4ccc(-c5ccc6ccc7ccccc7c6c5)cc4)c4ccc(-c5cc6ccccc6s5)cc4)cc-2c13]

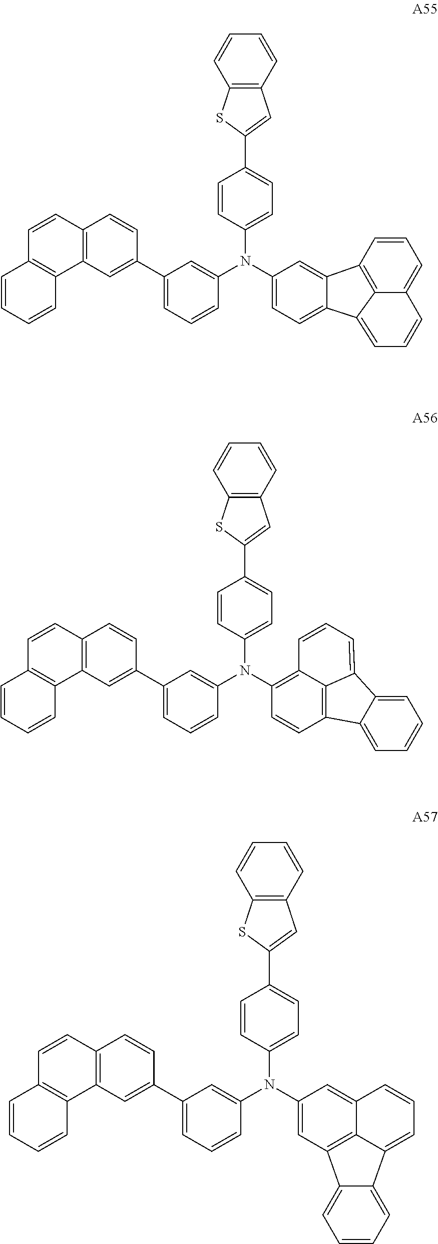 [SMILES: c1cc(-c2ccc3ccc4ccccc4c3c2)cc(N(c2ccc(-c3cc4ccccc4s3)cc2)c2ccc3c(c2)-c2cccc4cccc-3c24)c1 ; c1cc(-c2ccc3ccc4ccccc4c3c2)cc(N(c2ccc(-c3cc4ccccc4s3)cc2)c2ccc3c4c(cccc24)-c2ccccc2-3)c1 ; c1cc(-c2ccc3ccc4ccccc4c3c2)cc(N(c2ccc(-c3cc4ccccc4s3)cc2)c2cc3c4c(cccc4c2)-c2ccccc2-3)c1]

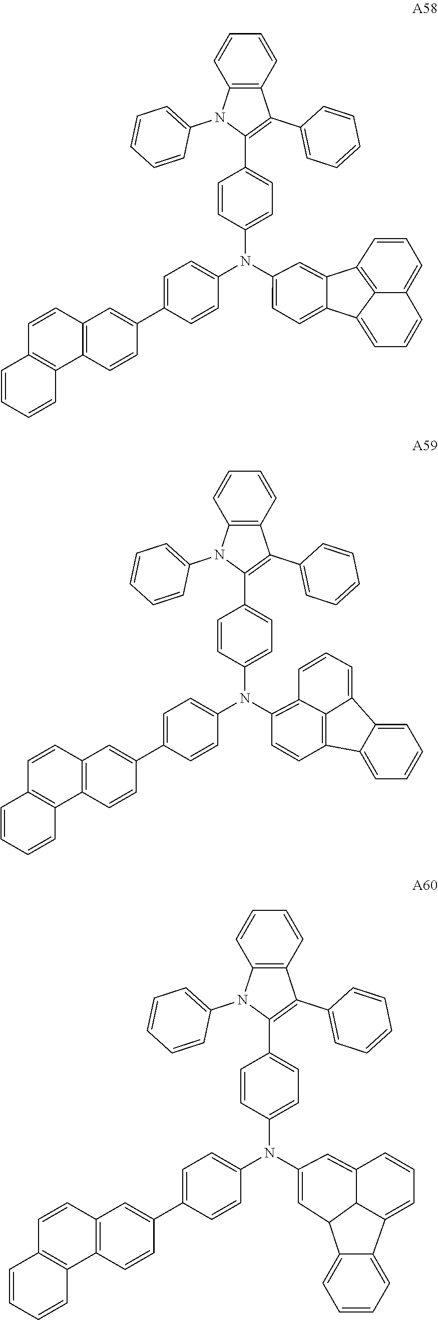 [SMILES: c1ccc(-c2c(-c3ccc(N(c4ccc(-c5ccc6c(ccc7ccccc76)c5)cc4)c4ccc5c(c4)-c4cccc6cccc-5c46)cc3)n(-c3ccccc3)c3ccccc23)cc1 ; c1ccc(-c2c(-c3ccc(N(c4ccc(-c5ccc6c(ccc7ccccc76)c5)cc4)c4ccc5c6c(cccc46)-c4ccccc4-5)cc3)n(-c3ccccc3)c3ccccc23)cc1 ; C1=CC2=CC(N(c3ccc(-c4ccc5c(ccc6ccccc65)c4)cc3)c3ccc(-c4c(-c5ccccc5)c5ccccc5n4-c4ccccc4)cc3)=CC3c4ccccc4C(=C1)C23]

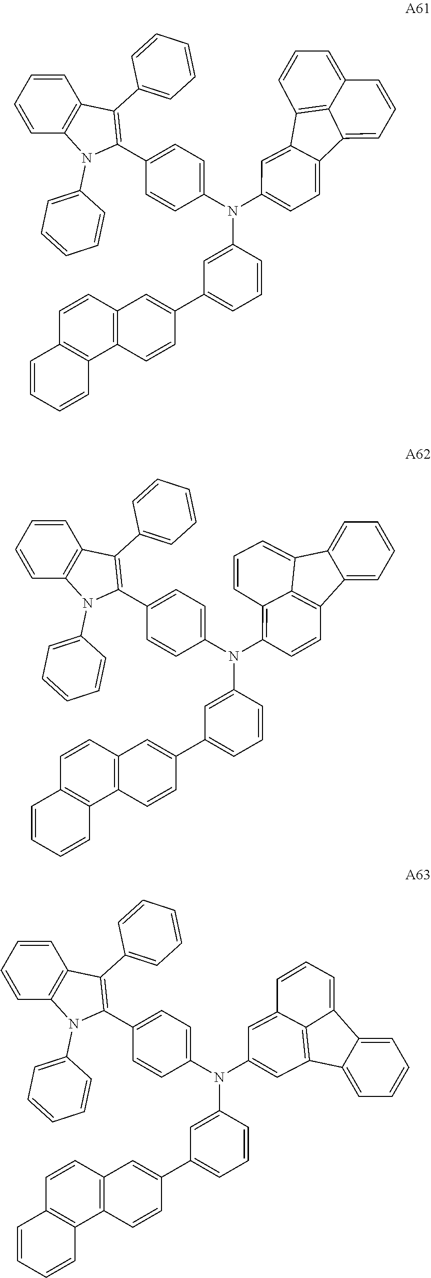 [SMILES: c1ccc(-c2c(-c3ccc(N(c4cccc(-c5ccc6c(ccc7ccccc76)c5)c4)c4ccc5c(c4)-c4cccc6cccc-5c46)cc3)n(-c3ccccc3)c3ccccc23)cc1 ; c1ccc(-c2c(-c3ccc(N(c4cccc(-c5ccc6c(ccc7ccccc76)c5)c4)c4ccc5c6c(cccc46)-c4ccccc4-5)cc3)n(-c3ccccc3)c3ccccc23)cc1 ; c1ccc(-c2c(-c3ccc(N(c4cccc(-c5ccc6c(ccc7ccccc76)c5)c4)c4cc5c6c(cccc6c4)-c4ccccc4-5)cc3)n(-c3ccccc3)c3ccccc23)cc1]

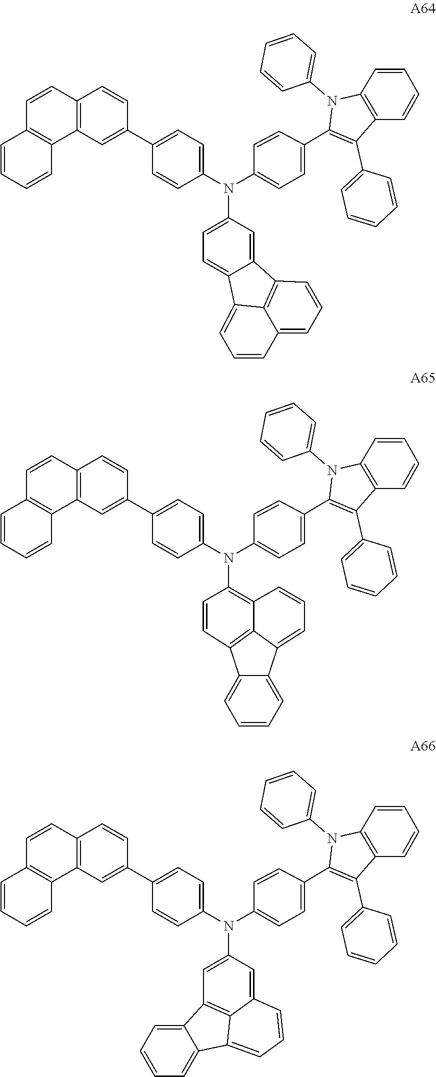 [SMILES: c1ccc(-c2c(-c3ccc(N(c4ccc(-c5ccc6ccc7ccccc7c6c5)cc4)c4ccc5c(c4)-c4cccc6cccc-5c46)cc3)n(-c3ccccc3)c3ccccc23)cc1 ; c1ccc(-c2c(-c3ccc(N(c4ccc(-c5ccc6ccc7ccccc7c6c5)cc4)c4ccc5c6c(cccc46)-c4ccccc4-5)cc3)n(-c3ccccc3)c3ccccc23)cc1 ; c1ccc(-c2c(-c3ccc(N(c4ccc(-c5ccc6ccc7ccccc7c6c5)cc4)c4cc5c6c(cccc6c4)-c4ccccc4-5)cc3)n(-c3ccccc3)c3ccccc23)cc1]

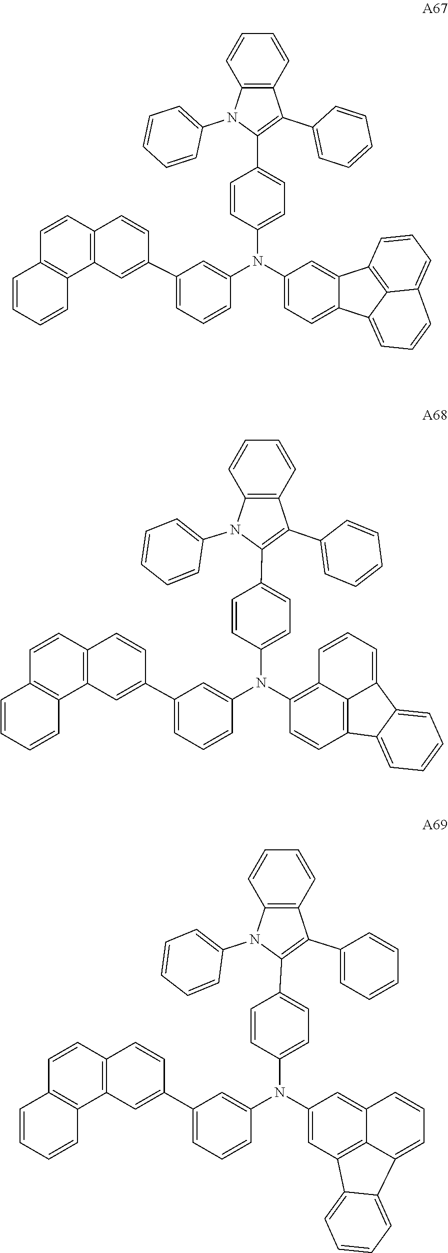 [SMILES: c1ccc(-c2c(-c3ccc(N(c4cccc(-c5ccc6ccc7ccccc7c6c5)c4)c4ccc5c(c4)-c4cccc6cccc-5c46)cc3)n(-c3ccccc3)c3ccccc23)cc1 ; c1ccc(-c2c(-c3ccc(N(c4cccc(-c5ccc6ccc7ccccc7c6c5)c4)c4ccc5c6c(cccc46)-c4ccccc4-5)cc3)n(-c3ccccc3)c3ccccc23)cc1 ; c1ccc(-c2c(-c3ccc(N(c4cccc(-c5ccc6ccc7ccccc7c6c5)c4)c4cc5c6c(cccc6c4)-c4ccccc4-5)cc3)n(-c3ccccc3)c3ccccc23)cc1]

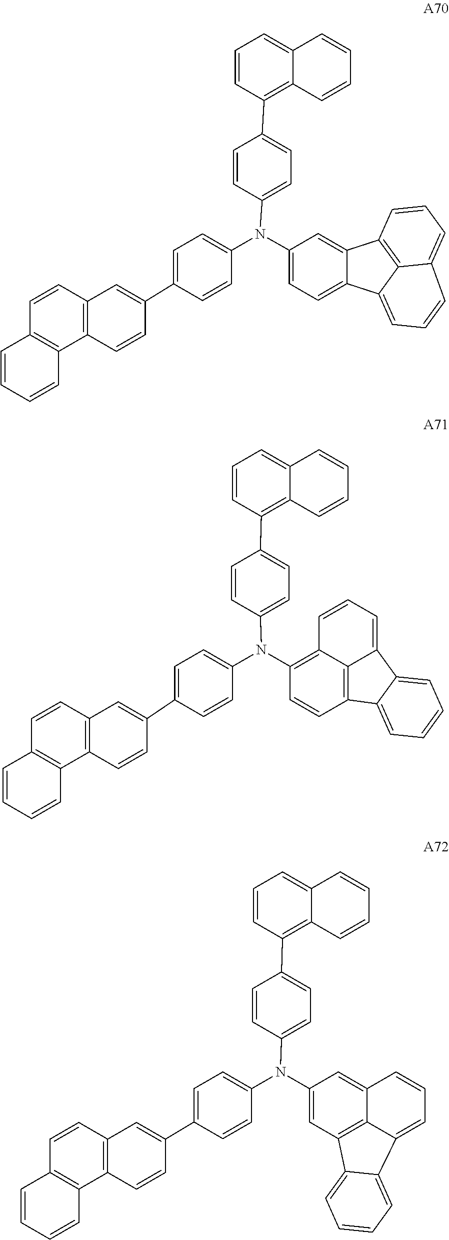 [SMILES: c1ccc2c(-c3ccc(N(c4ccc(-c5ccc6c(ccc7ccccc76)c5)cc4)c4ccc5c(c4)-c4cccc6cccc-5c46)cc3)cccc2c1 ; c1ccc2c(c1)-c1cccc3c(N(c4ccc(-c5ccc6c(ccc7ccccc76)c5)cc4)c4ccc(-c5cccc6ccccc56)cc4)ccc-2c13 ; c1ccc2c(c1)-c1cccc3cc(N(c4ccc(-c5ccc6c(ccc7ccccc76)c5)cc4)c4ccc(-c5cccc6ccccc56)cc4)cc-2c13]

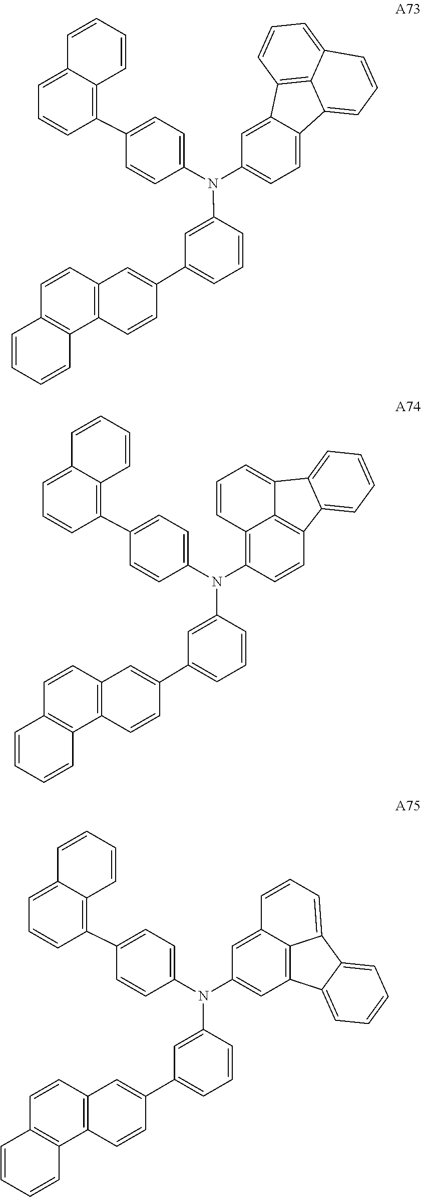 [SMILES: c1cc(-c2ccc3c(ccc4ccccc43)c2)cc(N(c2ccc(-c3cccc4ccccc34)cc2)c2ccc3c(c2)-c2cccc4cccc-3c24)c1 ; c1cc(-c2ccc3c(ccc4ccccc43)c2)cc(N(c2ccc(-c3cccc4ccccc34)cc2)c2ccc3c4c(cccc24)-c2ccccc2-3)c1 ; c1cc(-c2ccc3c(ccc4ccccc43)c2)cc(N(c2ccc(-c3cccc4ccccc34)cc2)c2cc3c4c(cccc4c2)-c2ccccc2-3)c1]

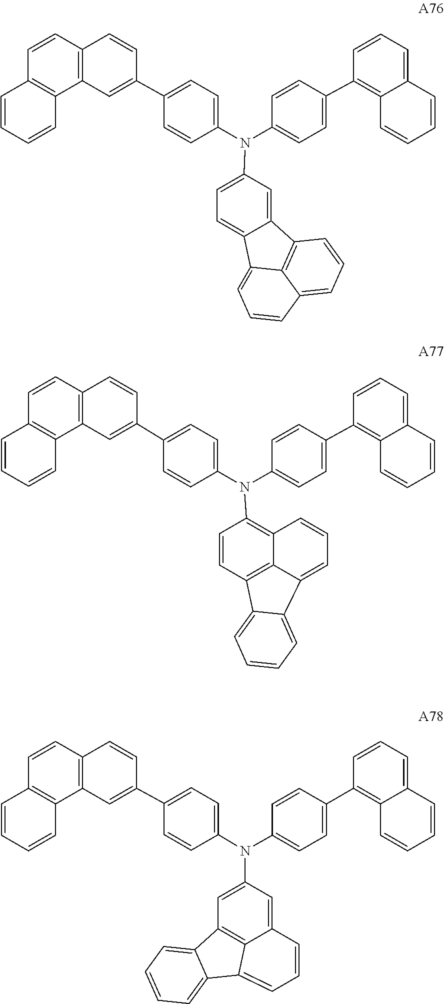 [SMILES: c1ccc2c(-c3ccc(N(c4ccc(-c5ccc6ccc7ccccc7c6c5)cc4)c4ccc5c(c4)-c4cccc6cccc-5c46)cc3)cccc2c1 ; c1ccc2c(c1)-c1cccc3c(N(c4ccc(-c5ccc6ccc7ccccc7c6c5)cc4)c4ccc(-c5cccc6ccccc56)cc4)ccc-2c13 ; c1ccc2c(c1)-c1cccc3cc(N(c4ccc(-c5ccc6ccc7ccccc7c6c5)cc4)c4ccc(-c5cccc6ccccc56)cc4)cc-2c13]

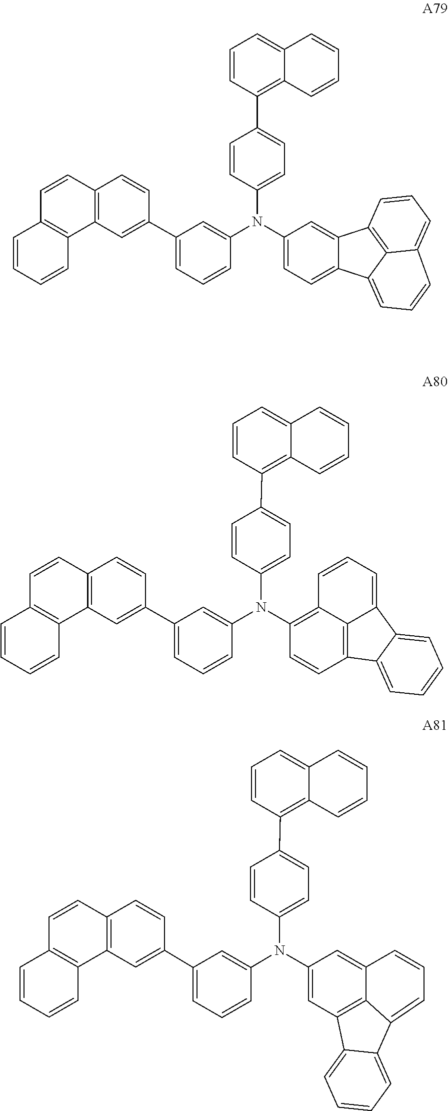 [SMILES: c1cc(-c2ccc3ccc4ccccc4c3c2)cc(N(c2ccc(-c3cccc4ccccc34)cc2)c2ccc3c(c2)-c2cccc4cccc-3c24)c1 ; c1cc(-c2ccc3ccc4ccccc4c3c2)cc(N(c2ccc(-c3cccc4ccccc34)cc2)c2ccc3c4c(cccc24)-c2ccccc2-3)c1 ; c1cc(-c2ccc3ccc4ccccc4c3c2)cc(N(c2ccc(-c3cccc4ccccc34)cc2)c2cc3c4c(cccc4c2)-c2ccccc2-3)c1]

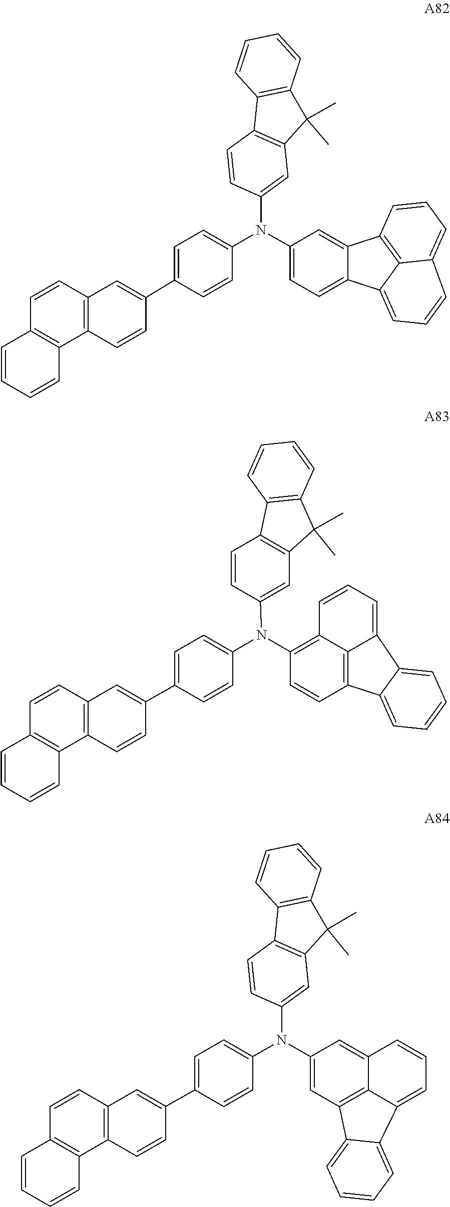 [SMILES: CC1(C)c2ccccc2-c2ccc(N(c3ccc(-c4ccc5c(ccc6ccccc65)c4)cc3)c3ccc4c(c3)-c3cccc5cccc-4c35)cc21 ; CC1(C)c2ccccc2-c2ccc(N(c3ccc(-c4ccc5c(ccc6ccccc65)c4)cc3)c3ccc4c5c(cccc35)-c3ccccc3-4)cc21 ; CC1(C)c2ccccc2-c2ccc(N(c3ccc(-c4ccc5c(ccc6ccccc65)c4)cc3)c3cc4c5c(cccc5c3)-c3ccccc3-4)cc21]

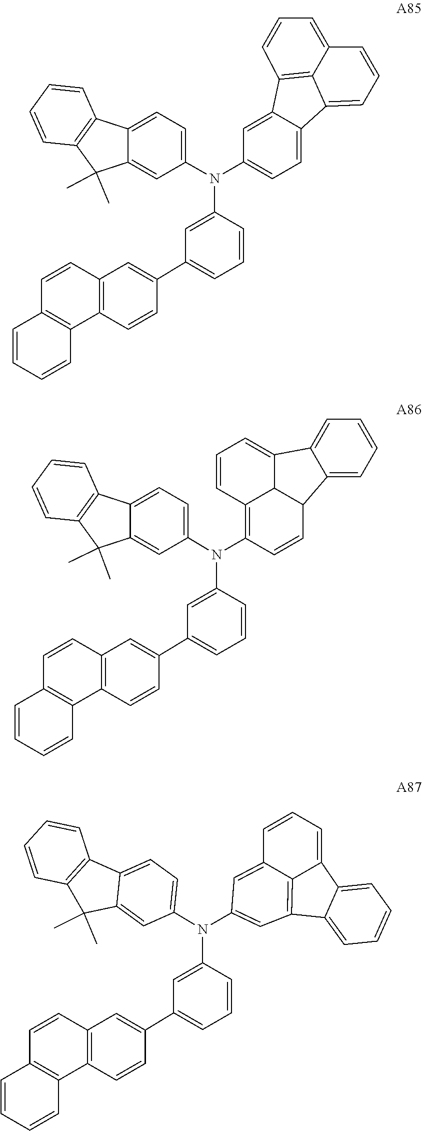 [SMILES: CC1(C)c2ccccc2-c2ccc(N(c3cccc(-c4ccc5c(ccc6ccccc65)c4)c3)c3ccc4c(c3)-c3cccc5cccc-4c35)cc21 ; CC1(C)c2ccccc2-c2ccc(N(C3=C4C=CC=C5c6ccccc6C(C=C3)C54)c3cccc(-c4ccc5c(ccc6ccccc65)c4)c3)cc21 ; CC1(C)c2ccccc2-c2ccc(N(c3cccc(-c4ccc5c(ccc6ccccc65)c4)c3)c3cc4c5c(cccc5c3)-c3ccccc3-4)cc21]

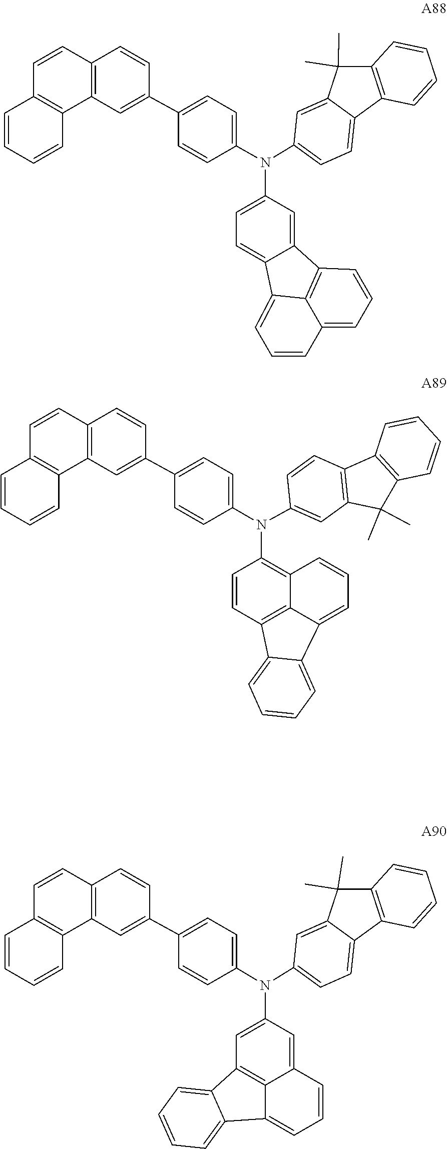 [SMILES: CC1(C)c2ccccc2-c2ccc(N(c3ccc(-c4ccc5ccc6ccccc6c5c4)cc3)c3ccc4c(c3)-c3cccc5cccc-4c35)cc21 ; CC1(C)c2ccccc2-c2ccc(N(c3ccc(-c4ccc5ccc6ccccc6c5c4)cc3)c3ccc4c5c(cccc35)-c3ccccc3-4)cc21 ; CC1(C)c2ccccc2-c2ccc(N(c3ccc(-c4ccc5ccc6ccccc6c5c4)cc3)c3cc4c5c(cccc5c3)-c3ccccc3-4)cc21]

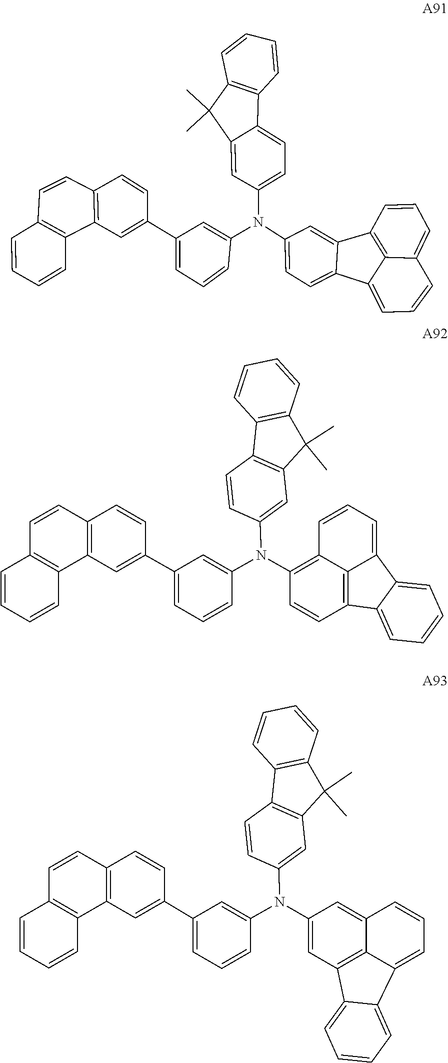 [SMILES: CC1(C)c2ccccc2-c2ccc(N(c3cccc(-c4ccc5ccc6ccccc6c5c4)c3)c3ccc4c(c3)-c3cccc5cccc-4c35)cc21 ; CC1(C)c2ccccc2-c2ccc(N(c3cccc(-c4ccc5ccc6ccccc6c5c4)c3)c3ccc4c5c(cccc35)-c3ccccc3-4)cc21 ; CC1(C)c2ccccc2-c2ccc(N(c3cccc(-c4ccc5ccc6ccccc6c5c4)c3)c3cc4c5c(cccc5c3)-c3ccccc3-4)cc21]

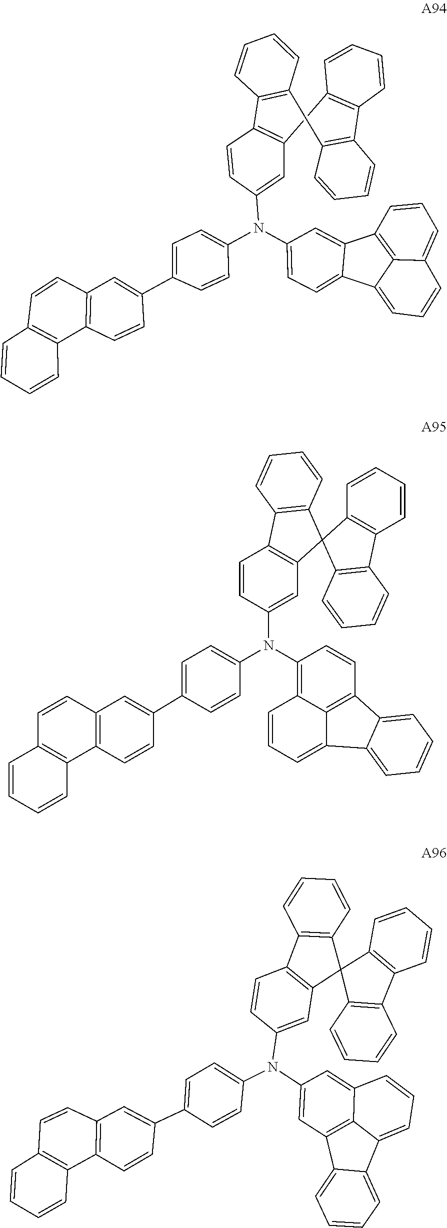 [SMILES: c1ccc2c(c1)-c1ccccc1-c1ccc(N(c3ccc(-c4ccc5c(ccc6ccccc65)c4)cc3)c3ccc4c(c3)-c3cccc5cccc-4c35)cc1-c1ccccc1-2 ; c1ccc2c(c1)-c1cccc3c(N(c4ccc(-c5ccc6c(ccc7ccccc76)c5)cc4)c4ccc5c(c4)C4(c6ccccc6-c6ccccc64)c4ccccc4-5)ccc-2c13 ; c1ccc2c(c1)-c1cccc3cc(N(c4ccc(-c5ccc6c(ccc7ccccc76)c5)cc4)c4ccc5c(c4)C4(c6ccccc6-c6ccccc64)c4ccccc4-5)cc-2c13]

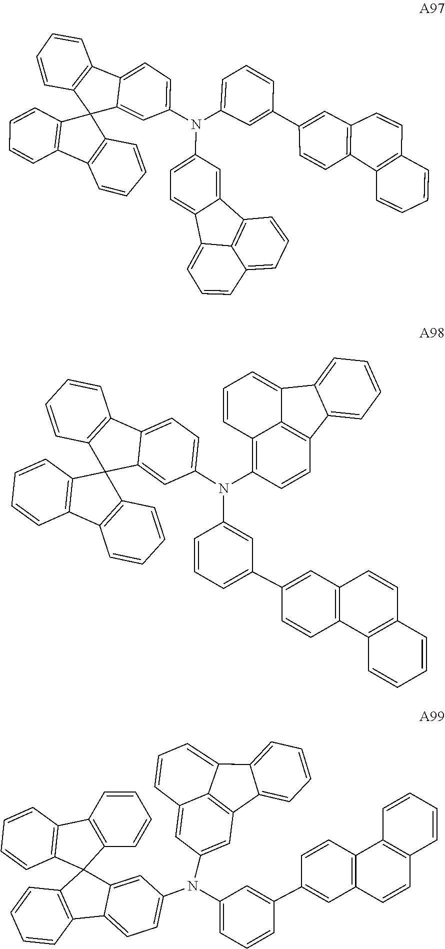 [SMILES: C=C1/C=C\c2ccccc2C/C=C\C(c2cccc(N(c3ccc4c(c3)-c3cccc5cccc-4c35)c3ccc4c(c3)C3(c5ccccc5-c5ccccc53)c3ccccc3-4)c2)=C/1 ; c1cc(-c2ccc3c(ccc4ccccc43)c2)cc(N(c2ccc3c(c2)C2(c4ccccc4-c4ccccc42)c2ccccc2-3)c2ccc3c4c(cccc24)-c2ccccc2-3)c1 ; c1cc(-c2ccc3c(ccc4ccccc43)c2)cc(N(c2ccc3c(c2)C2(c4ccccc4-c4ccccc42)c2ccccc2-3)c2cc3c4c(cccc4c2)-c2ccccc2-3)c1]

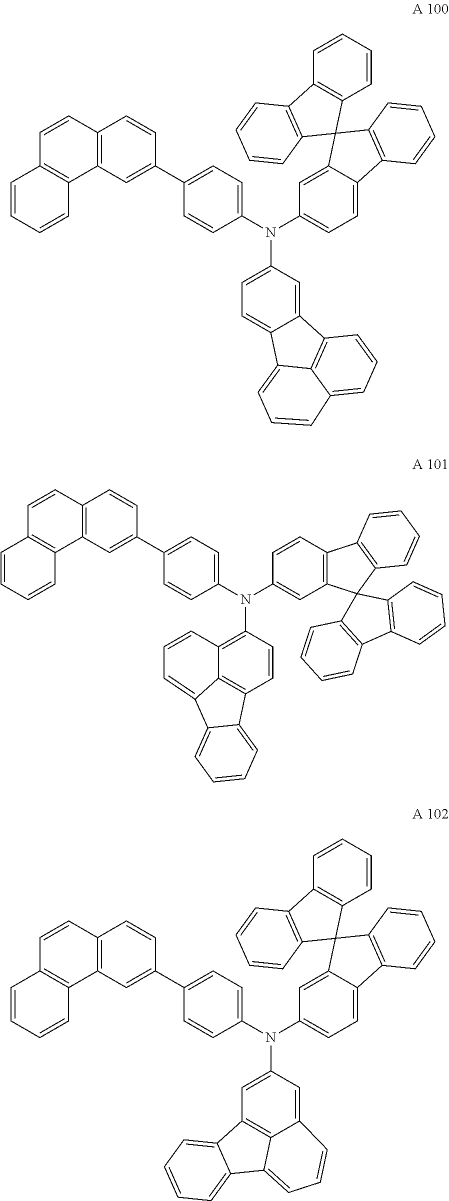 [SMILES: c1ccc2c(c1)-c1ccccc1C21c2ccccc2-c2ccc(N(c3ccc(-c4ccc5ccc6ccccc6c5c4)cc3)c3ccc4c(c3)-c3cccc5cccc-4c35)cc21 ; c1ccc2c(c1)-c1cccc3c(N(c4ccc(-c5ccc6ccc7ccccc7c6c5)cc4)c4ccc5c(c4)C4(c6ccccc6-c6ccccc64)c4ccccc4-5)ccc-2c13 ; c1ccc2c(c1)-c1cccc3cc(N(c4ccc(-c5ccc6ccc7ccccc7c6c5)cc4)c4ccc5c(c4)C4(c6ccccc6-c6ccccc64)c4ccccc4-5)cc-2c13]

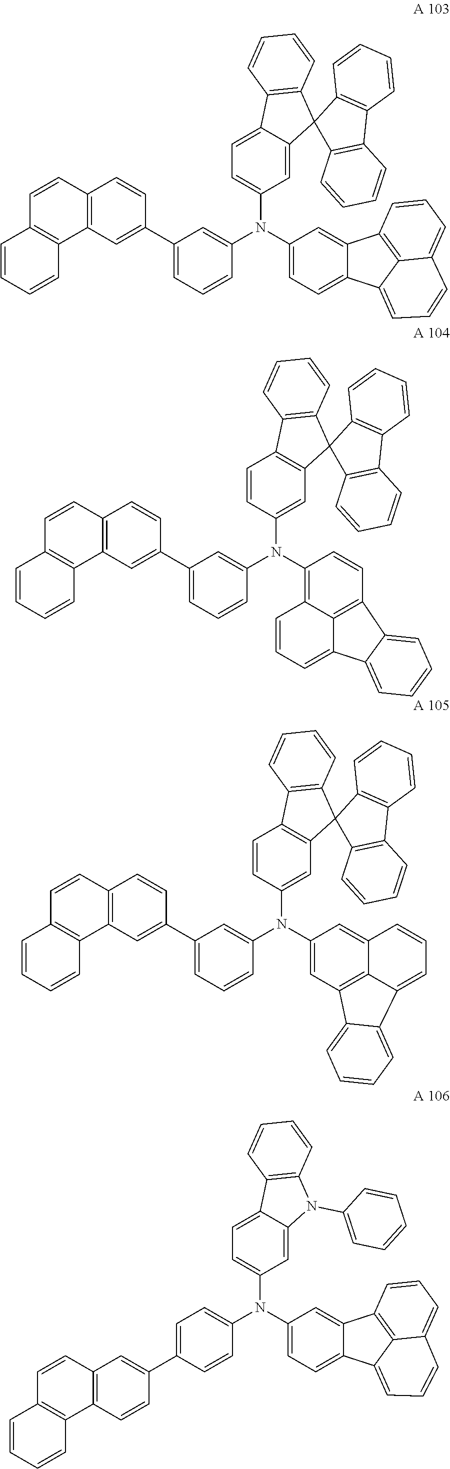 [SMILES: c1cc(-c2ccc3ccc4ccccc4c3c2)cc(N(c2ccc3c(c2)-c2cccc4cccc-3c24)c2ccc3c(c2)C2(c4ccccc4-c4ccccc42)c2ccccc2-3)c1 ; c1cc(-c2ccc3ccc4ccccc4c3c2)cc(N(c2ccc3c(c2)C2(c4ccccc4-c4ccccc42)c2ccccc2-3)c2ccc3c4c(cccc24)-c2ccccc2-3)c1 ; c1cc(-c2ccc3ccc4ccccc4c3c2)cc(N(c2ccc3c(c2)C2(c4ccccc4-c4ccccc42)c2ccccc2-3)c2cc3c4c(cccc4c2)-c2ccccc2-3)c1 ; c1ccc(-n2c3ccccc3c3ccc(N(c4ccc(-c5ccc6c(ccc7ccccc76)c5)cc4)c4ccc5c(c4)-c4cccc6cccc-5c46)cc32)cc1]

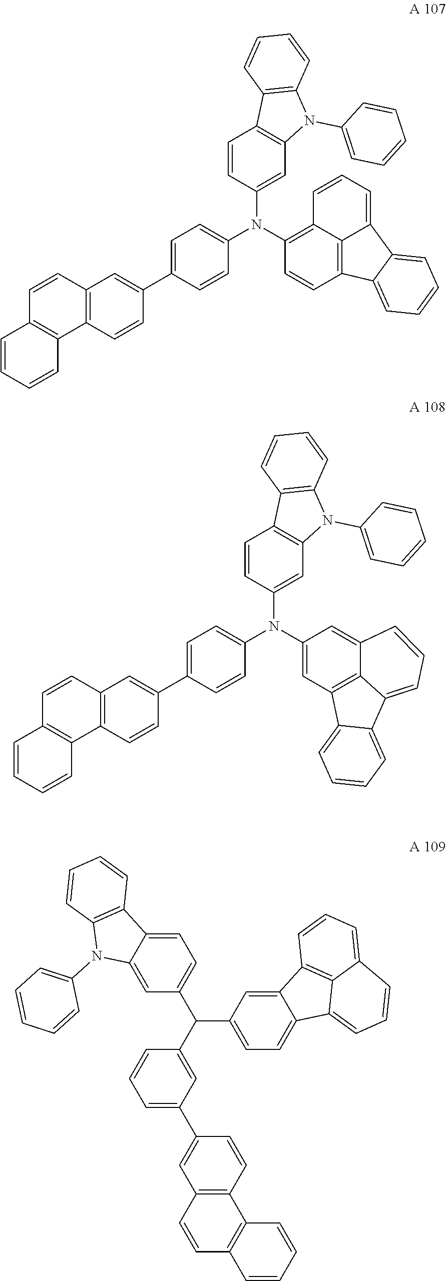 [SMILES: c1ccc(-n2c3ccccc3c3ccc(N(c4ccc(-c5ccc6c(ccc7ccccc76)c5)cc4)c4ccc5c6c(cccc46)-c4ccccc4-5)cc32)cc1 ; c1ccc(-n2c3ccccc3c3ccc(N(c4ccc(-c5ccc6c(ccc7ccccc76)c5)cc4)c4cc5c6c(cccc6c4)-c4ccccc4-5)cc32)cc1 ; c1ccc(-n2c3ccccc3c3ccc(C(c4cccc(-c5ccc6c(ccc7ccccc76)c5)c4)c4ccc5c(c4)-c4cccc6cccc-5c46)cc32)cc1]

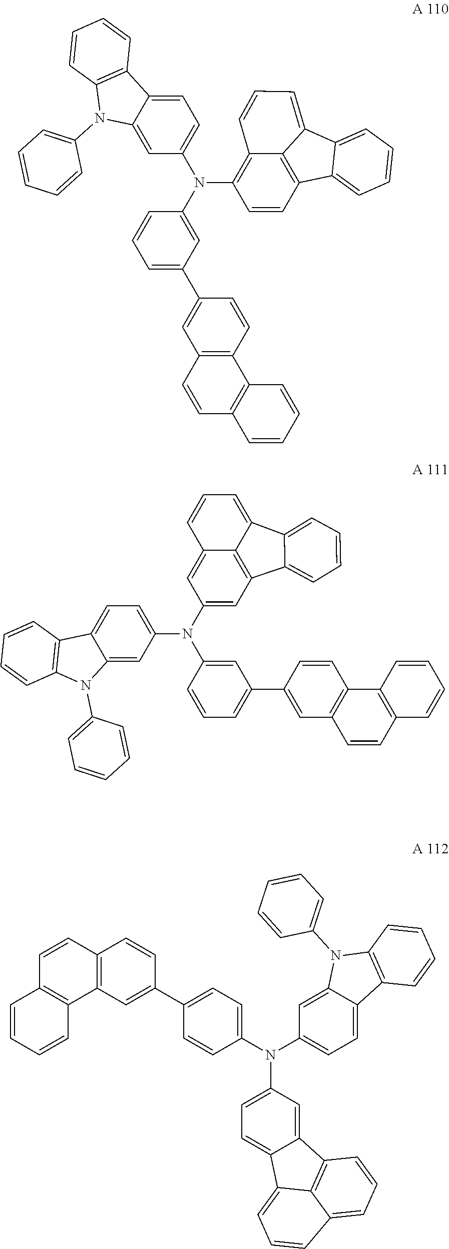 [SMILES: c1ccc(-n2c3ccccc3c3ccc(N(c4cccc(-c5ccc6c(ccc7ccccc76)c5)c4)c4ccc5c6c(cccc46)-c4ccccc4-5)cc32)cc1 ; c1ccc(-n2c3ccccc3c3ccc(N(c4cccc(-c5ccc6c(ccc7ccccc76)c5)c4)c4cc5c6c(cccc6c4)-c4ccccc4-5)cc32)cc1 ; c1ccc(-n2c3ccccc3c3ccc(N(c4ccc(-c5ccc6ccc7ccccc7c6c5)cc4)c4ccc5c(c4)-c4cccc6cccc-5c46)cc32)cc1]

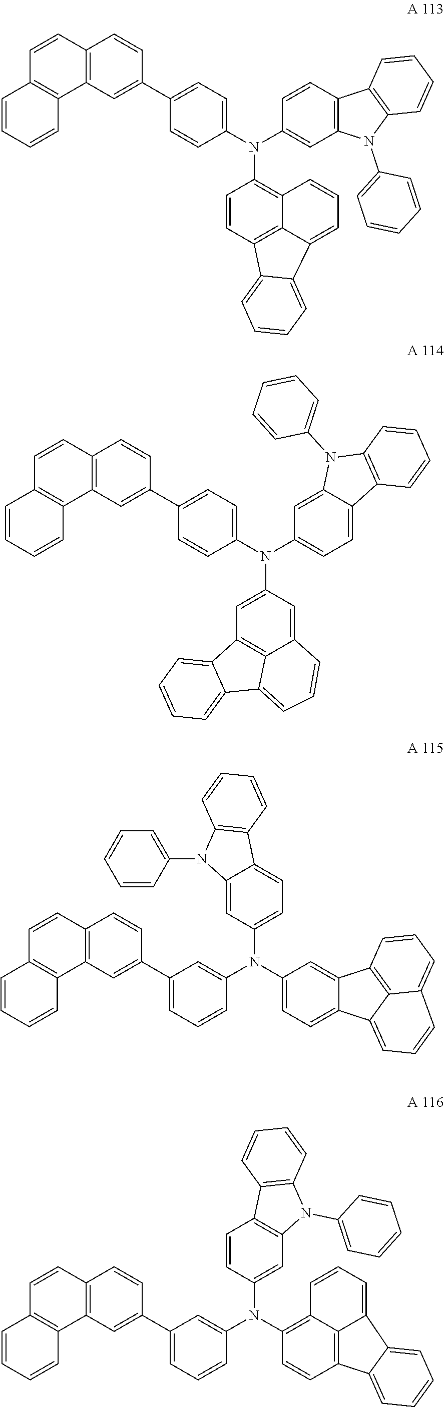

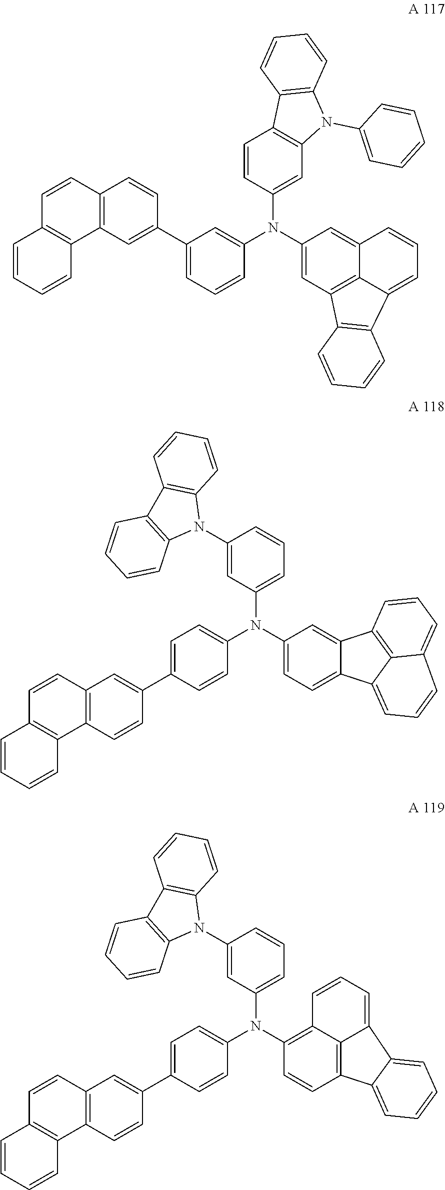 [SMILES: c1ccc(-n2c3ccccc3c3ccc(N(c4cccc(-c5ccc6ccc7ccccc7c6c5)c4)c4cc5c6c(cccc6c4)-c4ccccc4-5)cc32)cc1 ; c1cc(N(c2ccc(-c3ccc4c(ccc5ccccc54)c3)cc2)c2ccc3c(c2)-c2cccc4cccc-3c24)cc(-n2c3ccccc3c3ccccc32)c1 ; c1cc(N(c2ccc(-c3ccc4c(ccc5ccccc54)c3)cc2)c2ccc3c4c(cccc24)-c2ccccc2-3)cc(-n2c3ccccc3c3ccccc32)c1]

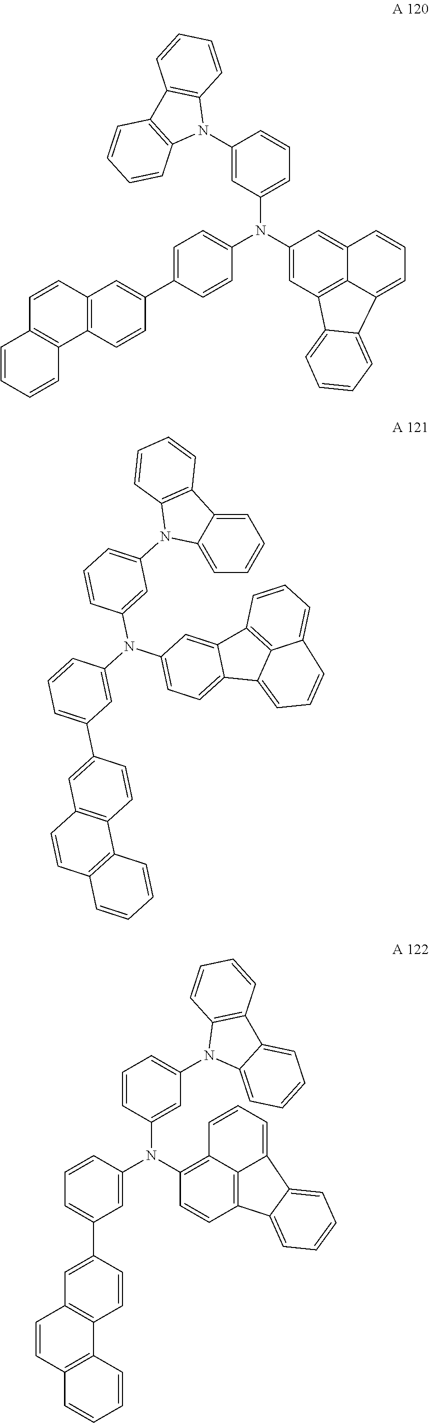 [SMILES: c1cc(N(c2ccc(-c3ccc4c(ccc5ccccc54)c3)cc2)c2cc3c4c(cccc4c2)-c2ccccc2-3)cc(-n2c3ccccc3c3ccccc32)c1 ; c1cc(-c2ccc3c(ccc4ccccc43)c2)cc(N(c2cccc(-n3c4ccccc4c4ccccc43)c2)c2ccc3c(c2)-c2cccc4cccc-3c24)c1 ; c1cc(-c2ccc3c(ccc4ccccc43)c2)cc(N(c2cccc(-n3c4ccccc4c4ccccc43)c2)c2ccc3c4c(cccc24)-c2ccccc2-3)c1]

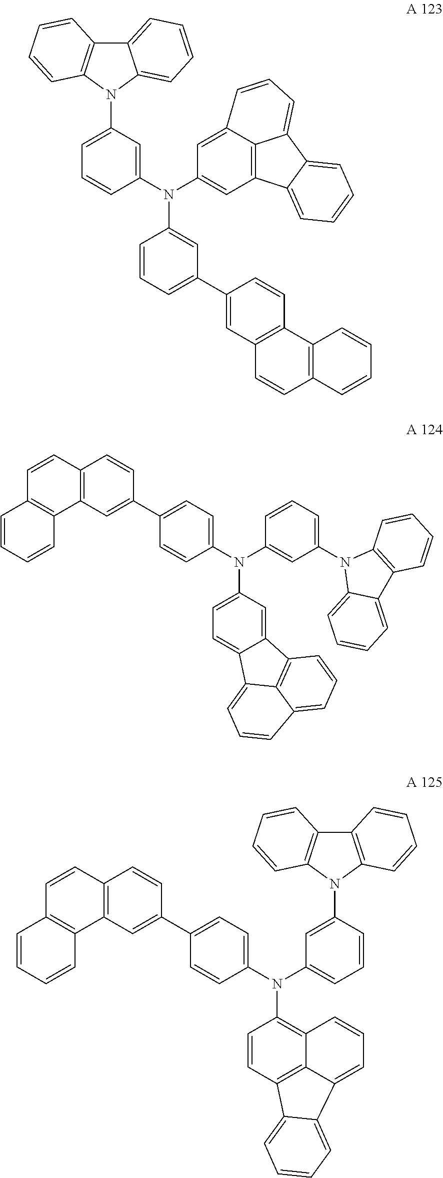 [SMILES: c1cc(-c2ccc3c(ccc4ccccc43)c2)cc(N(c2cccc(-n3c4ccccc4c4ccccc43)c2)c2cc3c4c(cccc4c2)-c2ccccc2-3)c1 ; c1cc(N(c2ccc(-c3ccc4ccc5ccccc5c4c3)cc2)c2ccc3c(c2)-c2cccc4cccc-3c24)cc(-n2c3ccccc3c3ccccc32)c1 ; c1cc(N(c2ccc(-c3ccc4ccc5ccccc5c4c3)cc2)c2ccc3c4c(cccc24)-c2ccccc2-3)cc(-n2c3ccccc3c3ccccc32)c1]

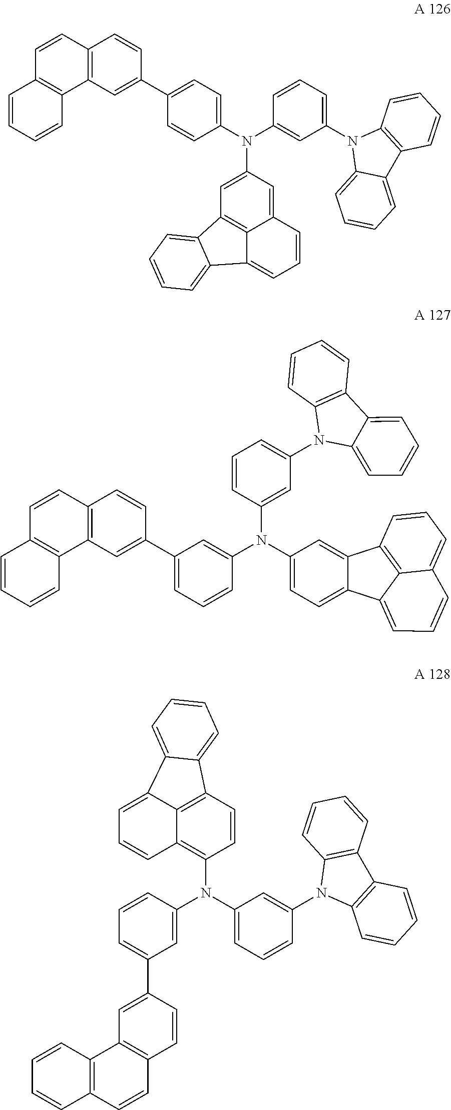 [SMILES: c1cc(N(c2ccc(-c3ccc4ccc5ccccc5c4c3)cc2)c2cc3c4c(cccc4c2)-c2ccccc2-3)cc(-n2c3ccccc3c3ccccc32)c1 ; c1cc(-c2ccc3ccc4ccccc4c3c2)cc(N(c2cccc(-n3c4ccccc4c4ccccc43)c2)c2ccc3c(c2)-c2cccc4cccc-3c24)c1 ; c1cc(-c2ccc3ccc4ccccc4c3c2)cc(N(c2cccc(-n3c4ccccc4c4ccccc43)c2)c2ccc3c4c(cccc24)-c2ccccc2-3)c1]

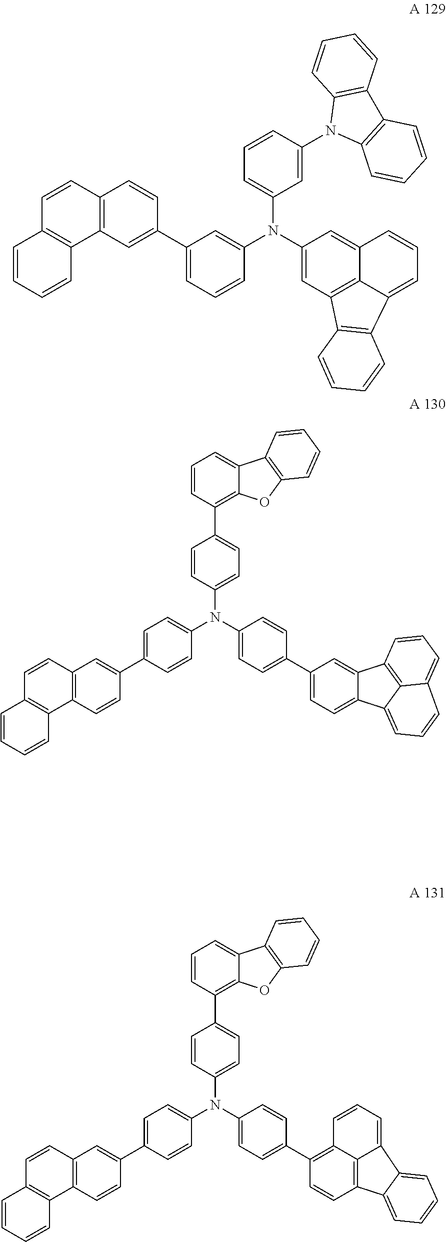 [SMILES: c1cc(-c2ccc3ccc4ccccc4c3c2)cc(N(c2cccc(-n3c4ccccc4c4ccccc43)c2)c2cc3c4c(cccc4c2)-c2ccccc2-3)c1 ; c1cc2c3c(cccc3c1)-c1cc(-c3ccc(N(c4ccc(-c5ccc6c(ccc7ccccc76)c5)cc4)c4ccc(-c5cccc6c5oc5ccccc56)cc4)cc3)ccc1-2 ; c1ccc2c(c1)-c1cccc3c(-c4ccc(N(c5ccc(-c6ccc7c(ccc8ccccc87)c6)cc5)c5ccc(-c6cccc7c6oc6ccccc67)cc5)cc4)ccc-2c13]

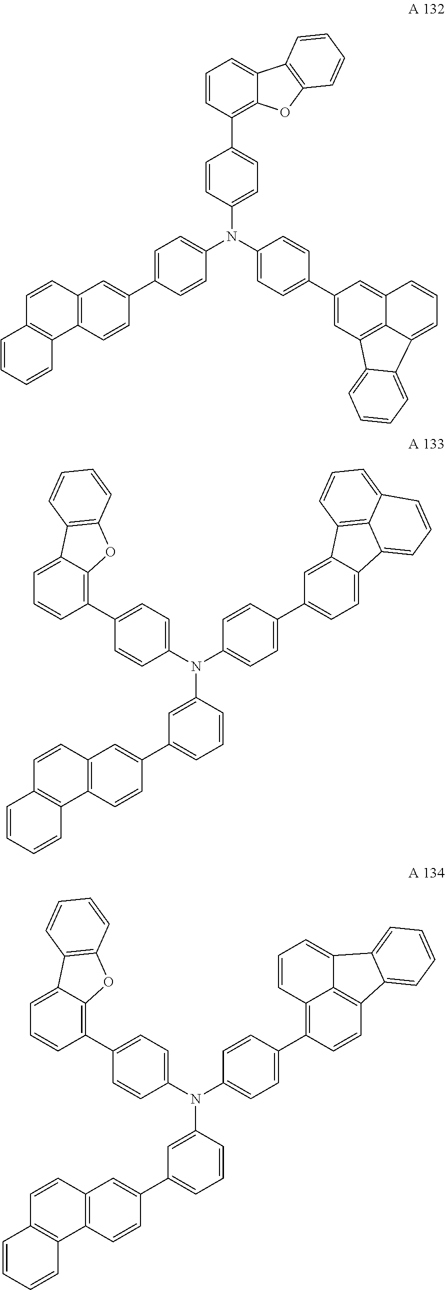 [SMILES: c1ccc2c(c1)-c1cccc3cc(-c4ccc(N(c5ccc(-c6ccc7c(ccc8ccccc87)c6)cc5)c5ccc(-c6cccc7c6oc6ccccc67)cc5)cc4)cc-2c13 ; c1cc(-c2ccc3c(ccc4ccccc43)c2)cc(N(c2ccc(-c3ccc4c(c3)-c3cccc5cccc-4c35)cc2)c2ccc(-c3cccc4c3oc3ccccc34)cc2)c1 ; c1cc(-c2ccc3c(ccc4ccccc43)c2)cc(N(c2ccc(-c3ccc4c5c(cccc35)-c3ccccc3-4)cc2)c2ccc(-c3cccc4c3oc3ccccc34)cc2)c1]

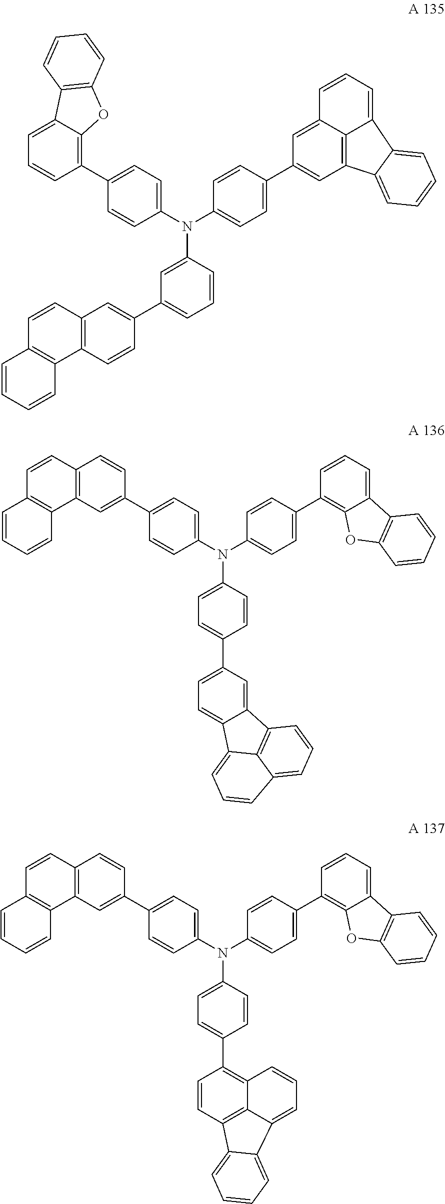 [SMILES: c1cc(-c2ccc3c(ccc4ccccc43)c2)cc(N(c2ccc(-c3cc4c5c(cccc5c3)-c3ccccc3-4)cc2)c2ccc(-c3cccc4c3oc3ccccc34)cc2)c1 ; c1cc2c3c(cccc3c1)-c1cc(-c3ccc(N(c4ccc(-c5ccc6ccc7ccccc7c6c5)cc4)c4ccc(-c5cccc6c5oc5ccccc56)cc4)cc3)ccc1-2 ; c1ccc2c(c1)-c1cccc3c(-c4ccc(N(c5ccc(-c6ccc7ccc8ccccc8c7c6)cc5)c5ccc(-c6cccc7c6oc6ccccc67)cc5)cc4)ccc-2c13]

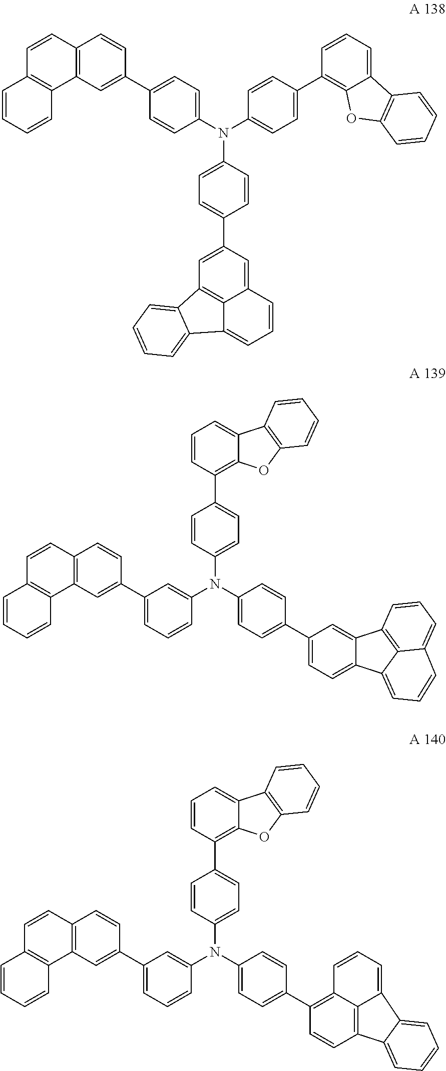 [SMILES: c1ccc2c(c1)-c1cccc3cc(-c4ccc(N(c5ccc(-c6ccc7ccc8ccccc8c7c6)cc5)c5ccc(-c6cccc7c6oc6ccccc67)cc5)cc4)cc-2c13 ; c1cc(-c2ccc3ccc4ccccc4c3c2)cc(N(c2ccc(-c3ccc4c(c3)-c3cccc5cccc-4c35)cc2)c2ccc(-c3cccc4c3oc3ccccc34)cc2)c1 ; c1cc(-c2ccc3ccc4ccccc4c3c2)cc(N(c2ccc(-c3ccc4c5c(cccc35)-c3ccccc3-4)cc2)c2ccc(-c3cccc4c3oc3ccccc34)cc2)c1]

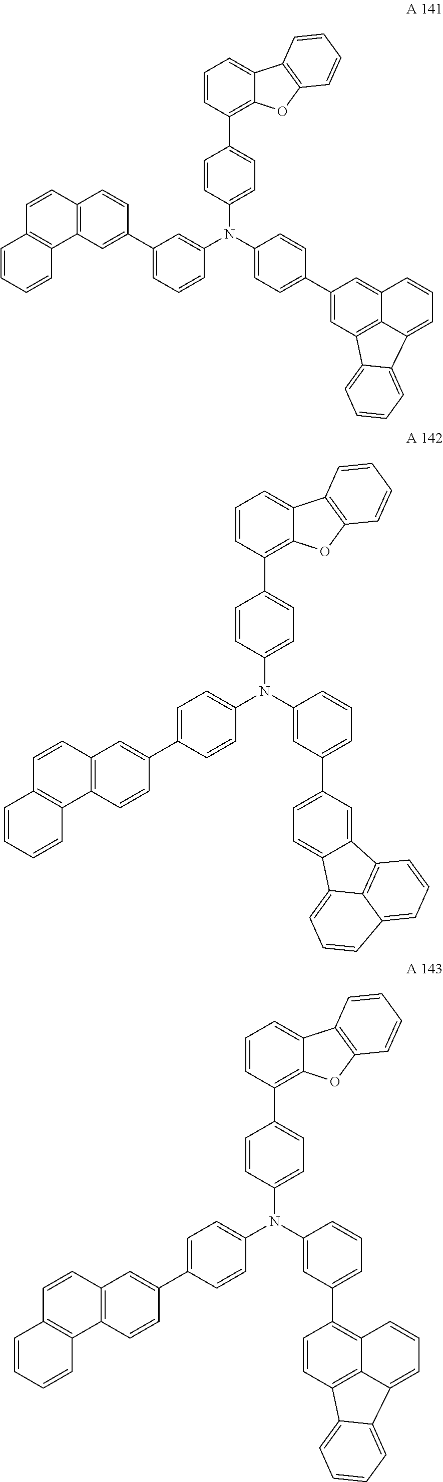

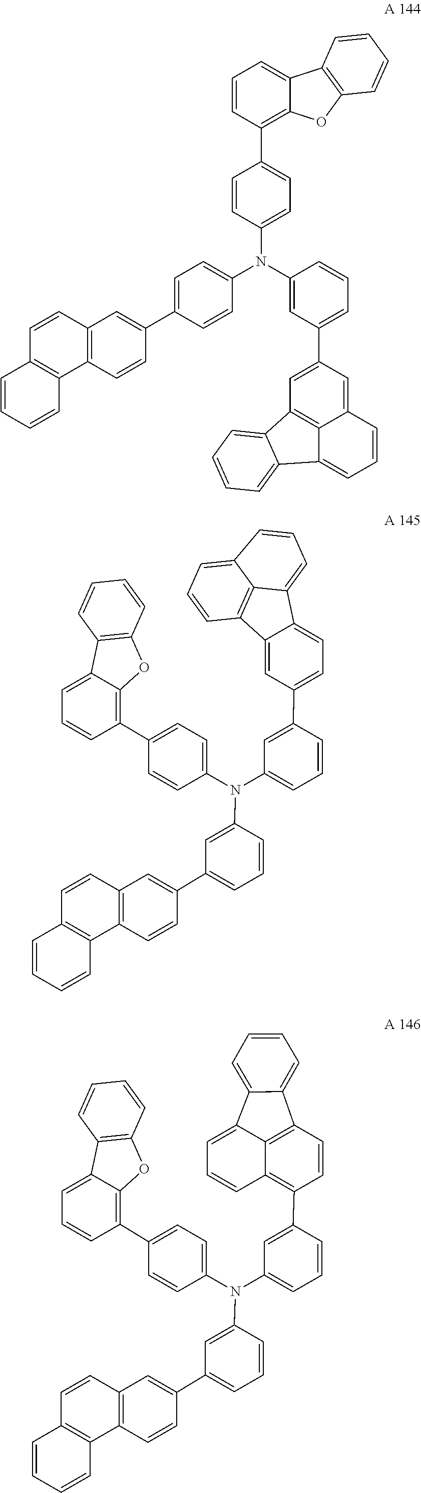

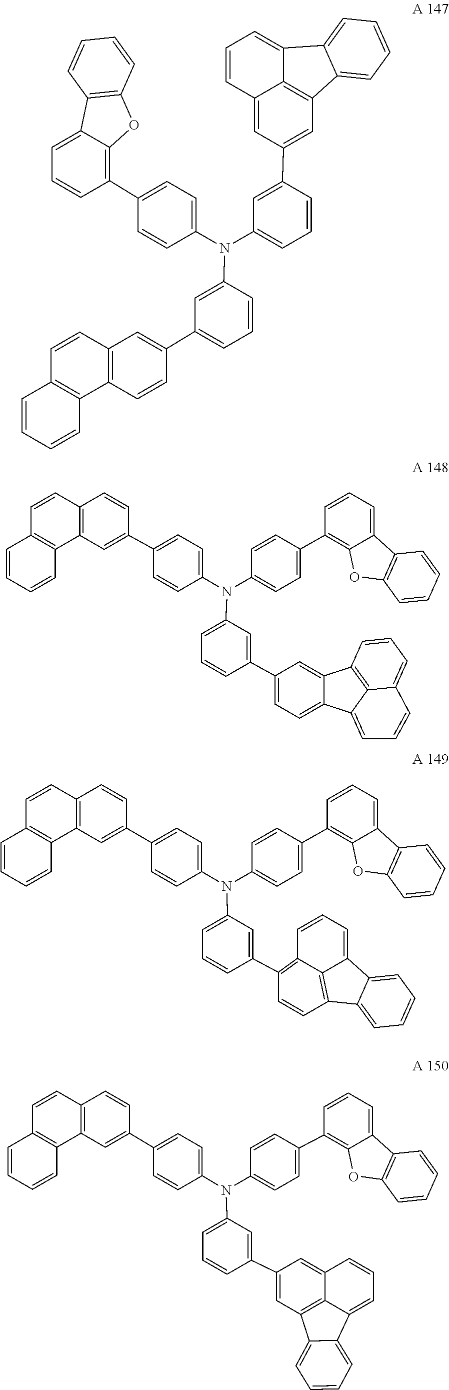 [SMILES: c1cc(-c2cc3c4c(cccc4c2)-c2ccccc2-3)cc(N(c2ccc(-c3cccc4c3oc3ccccc34)cc2)c2cccc(-c3ccc4c(ccc5ccccc54)c3)c2)c1 ; c1cc(-c2ccc3c(c2)-c2cccc4cccc-3c24)cc(N(c2ccc(-c3ccc4ccc5ccccc5c4c3)cc2)c2ccc(-c3cccc4c3oc3ccccc34)cc2)c1 ; c1cc(-c2ccc3c4c(cccc24)-c2ccccc2-3)cc(N(c2ccc(-c3ccc4ccc5ccccc5c4c3)cc2)c2ccc(-c3cccc4c3oc3ccccc34)cc2)c1 ; c1cc(-c2cc3c4c(cccc4c2)-c2ccccc2-3)cc(N(c2ccc(-c3ccc4ccc5ccccc5c4c3)cc2)c2ccc(-c3cccc4c3oc3ccccc34)cc2)c1]

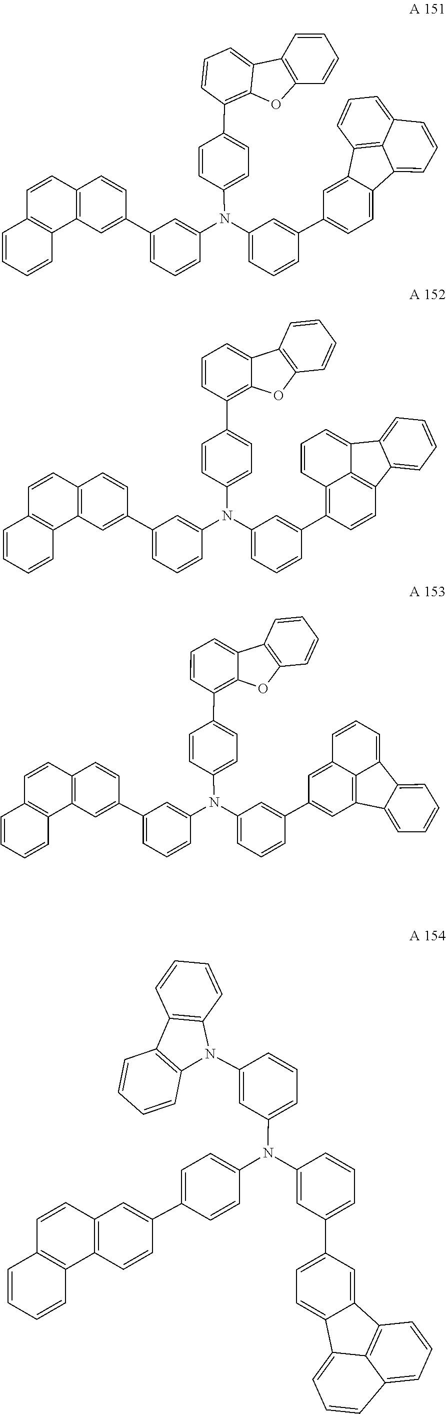 [SMILES: c1cc(-c2ccc3c(c2)-c2cccc4cccc-3c24)cc(N(c2ccc(-c3cccc4c3oc3ccccc34)cc2)c2cccc(-c3ccc4ccc5ccccc5c4c3)c2)c1 ; c1cc(-c2ccc3ccc4ccccc4c3c2)cc(N(c2ccc(-c3cccc4c3oc3ccccc34)cc2)c2cccc(-c3ccc4c5c(cccc35)-c3ccccc3-4)c2)c1 ; c1cc(-c2cc3c4c(cccc4c2)-c2ccccc2-3)cc(N(c2ccc(-c3cccc4c3oc3ccccc34)cc2)c2cccc(-c3ccc4ccc5ccccc5c4c3)c2)c1 ; c1cc(-c2ccc3c(c2)-c2cccc4cccc-3c24)cc(N(c2ccc(-c3ccc4c(ccc5ccccc54)c3)cc2)c2cccc(-n3c4ccccc4c4ccccc43)c2)c1]

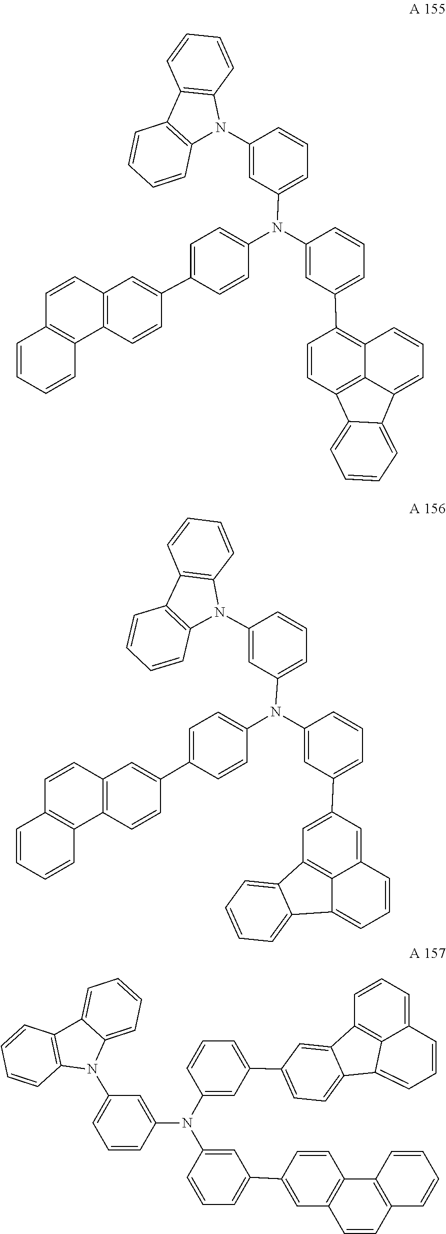 [SMILES: c1cc(-c2ccc3c4c(cccc24)-c2ccccc2-3)cc(N(c2ccc(-c3ccc4c(ccc5ccccc54)c3)cc2)c2cccc(-n3c4ccccc4c4ccccc43)c2)c1 ; c1cc(-c2cc3c4c(cccc4c2)-c2ccccc2-3)cc(N(c2ccc(-c3ccc4c(ccc5ccccc54)c3)cc2)c2cccc(-n3c4ccccc4c4ccccc43)c2)c1 ; c1cc(-c2ccc3c(c2)-c2cccc4cccc-3c24)cc(N(c2cccc(-c3ccc4c(ccc5ccccc54)c3)c2)c2cccc(-n3c4ccccc4c4ccccc43)c2)c1]

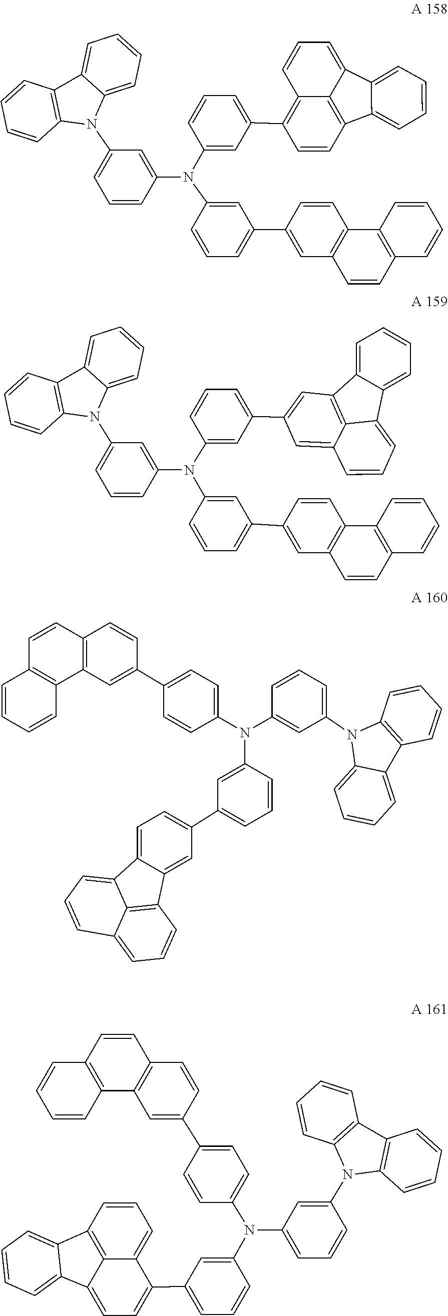 [SMILES: c1cc(-c2ccc3c(ccc4ccccc43)c2)cc(N(c2cccc(-c3ccc4c5c(cccc35)-c3ccccc3-4)c2)c2cccc(-n3c4ccccc4c4ccccc43)c2)c1 ; c1cc(-c2cc3c4c(cccc4c2)-c2ccccc2-3)cc(N(c2cccc(-c3ccc4c(ccc5ccccc54)c3)c2)c2cccc(-n3c4ccccc4c4ccccc43)c2)c1 ; c1cc(-c2ccc3c(c2)-c2cccc4cccc-3c24)cc(N(c2ccc(-c3ccc4ccc5ccccc5c4c3)cc2)c2cccc(-n3c4ccccc4c4ccccc43)c2)c1 ; c1cc(-c2ccc3c4c(cccc24)-c2ccccc2-3)cc(N(c2ccc(-c3ccc4ccc5ccccc5c4c3)cc2)c2cccc(-n3c4ccccc4c4ccccc43)c2)c1]

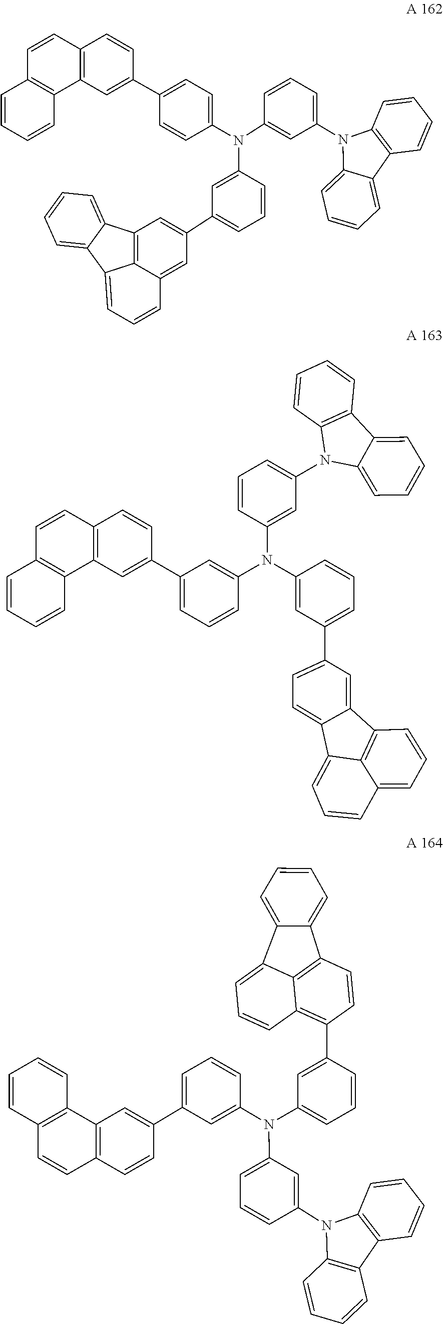 [SMILES: c1cc(-c2cc3c4c(cccc4c2)-c2ccccc2-3)cc(N(c2ccc(-c3ccc4ccc5ccccc5c4c3)cc2)c2cccc(-n3c4ccccc4c4ccccc43)c2)c1 ; c1cc(-c2ccc3c(c2)-c2cccc4cccc-3c24)cc(N(c2cccc(-c3ccc4ccc5ccccc5c4c3)c2)c2cccc(-n3c4ccccc4c4ccccc43)c2)c1 ; c1cc(-c2ccc3ccc4ccccc4c3c2)cc(N(c2cccc(-c3ccc4c5c(cccc35)-c3ccccc3-4)c2)c2cccc(-n3c4ccccc4c4ccccc43)c2)c1]

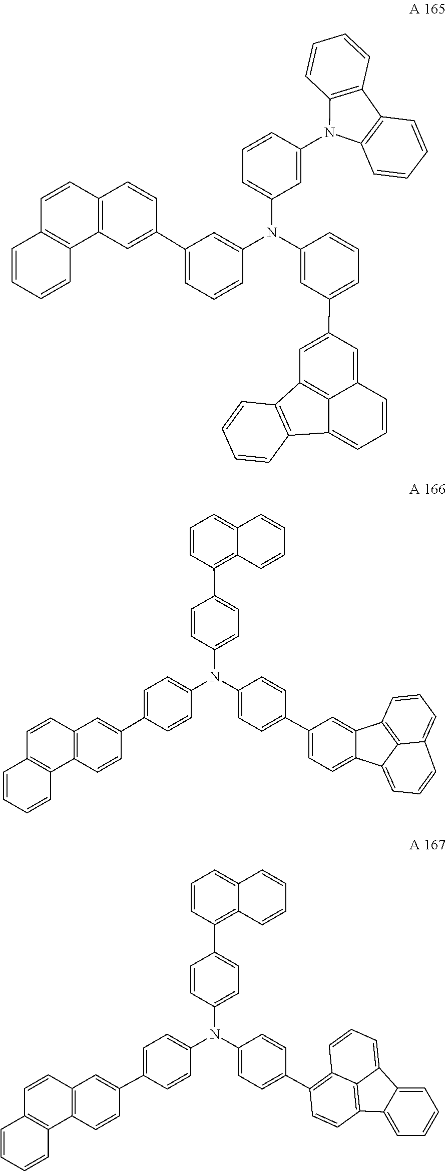 [SMILES: c1cc(-c2cc3c4c(cccc4c2)-c2ccccc2-3)cc(N(c2cccc(-c3ccc4ccc5ccccc5c4c3)c2)c2cccc(-n3c4ccccc4c4ccccc43)c2)c1 ; c1ccc2c(-c3ccc(N(c4ccc(-c5ccc6c(c5)-c5cccc7cccc-6c57)cc4)c4ccc(-c5ccc6c(ccc7ccccc76)c5)cc4)cc3)cccc2c1 ; c1ccc2c(c1)-c1cccc3c(-c4ccc(N(c5ccc(-c6ccc7c(ccc8ccccc87)c6)cc5)c5ccc(-c6cccc7ccccc67)cc5)cc4)ccc-2c13]

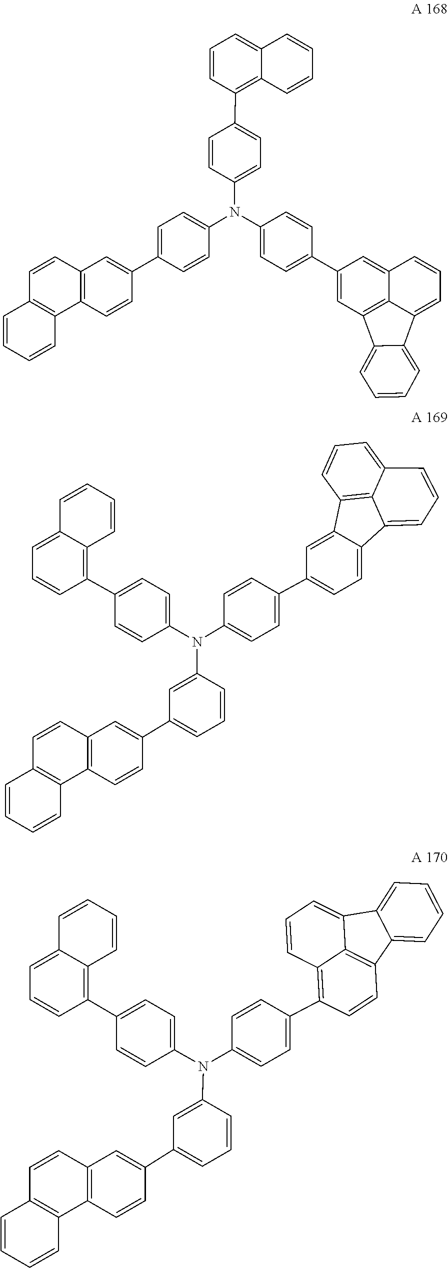 [SMILES: c1ccc2c(c1)-c1cccc3cc(-c4ccc(N(c5ccc(-c6ccc7c(ccc8ccccc87)c6)cc5)c5ccc(-c6cccc7ccccc67)cc5)cc4)cc-2c13 ; c1cc(-c2ccc3c(ccc4ccccc43)c2)cc(N(c2ccc(-c3ccc4c(c3)-c3cccc5cccc-4c35)cc2)c2ccc(-c3cccc4ccccc34)cc2)c1 ; c1cc(-c2ccc3c(ccc4ccccc43)c2)cc(N(c2ccc(-c3cccc4ccccc34)cc2)c2ccc(-c3ccc4c5c(cccc35)-c3ccccc3-4)cc2)c1]

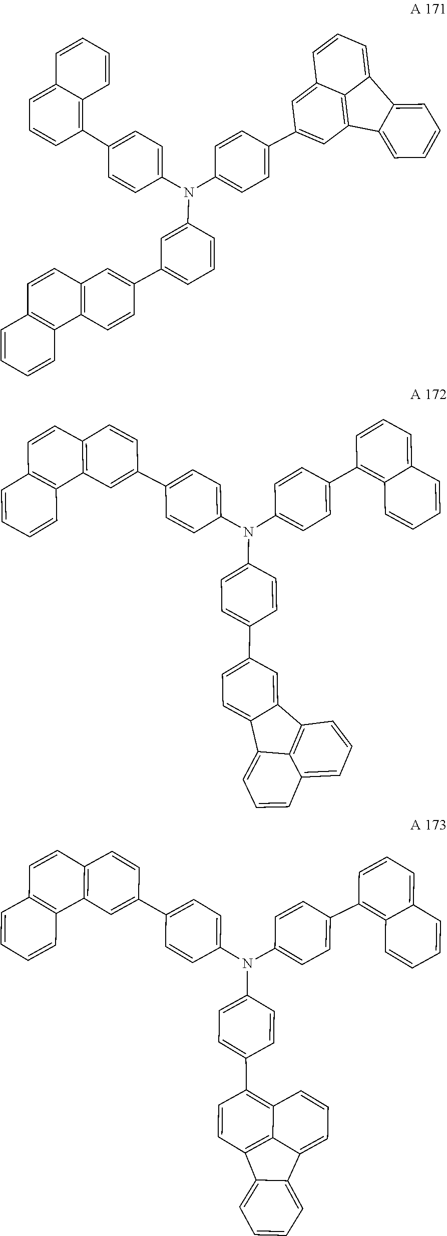 [SMILES: c1cc(-c2ccc3c(ccc4ccccc43)c2)cc(N(c2ccc(-c3cc4c5c(cccc5c3)-c3ccccc3-4)cc2)c2ccc(-c3cccc4ccccc34)cc2)c1 ; c1ccc2c(-c3ccc(N(c4ccc(-c5ccc6c(c5)-c5cccc7cccc-6c57)cc4)c4ccc(-c5ccc6ccc7ccccc7c6c5)cc4)cc3)cccc2c1 ; c1ccc2c(c1)-c1cccc3c(-c4ccc(N(c5ccc(-c6ccc7ccc8ccccc8c7c6)cc5)c5ccc(-c6cccc7ccccc67)cc5)cc4)ccc-2c13]

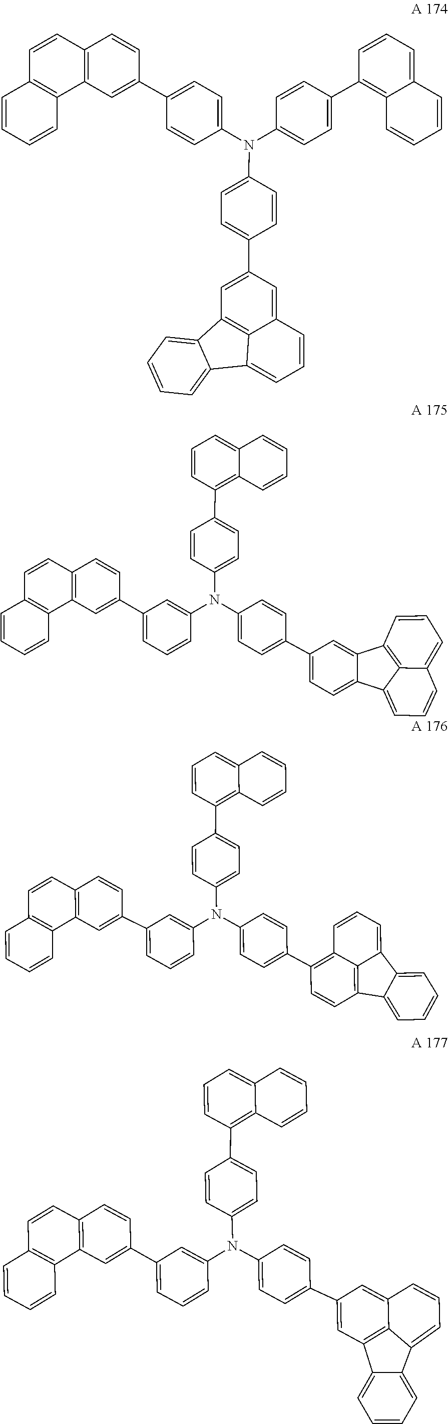 [SMILES: c1ccc2c(c1)-c1cccc3cc(-c4ccc(N(c5ccc(-c6ccc7ccc8ccccc8c7c6)cc5)c5ccc(-c6cccc7ccccc67)cc5)cc4)cc-2c13 ; c1cc(-c2ccc3ccc4ccccc4c3c2)cc(N(c2ccc(-c3ccc4c(c3)-c3cccc5cccc-4c35)cc2)c2ccc(-c3cccc4ccccc34)cc2)c1 ; c1cc(-c2ccc3ccc4ccccc4c3c2)cc(N(c2ccc(-c3cccc4ccccc34)cc2)c2ccc(-c3ccc4c5c(cccc35)-c3ccccc3-4)cc2)c1 ; c1cc(-c2ccc3ccc4ccccc4c3c2)cc(N(c2ccc(-c3cc4c5c(cccc5c3)-c3ccccc3-4)cc2)c2ccc(-c3cccc4ccccc34)cc2)c1]

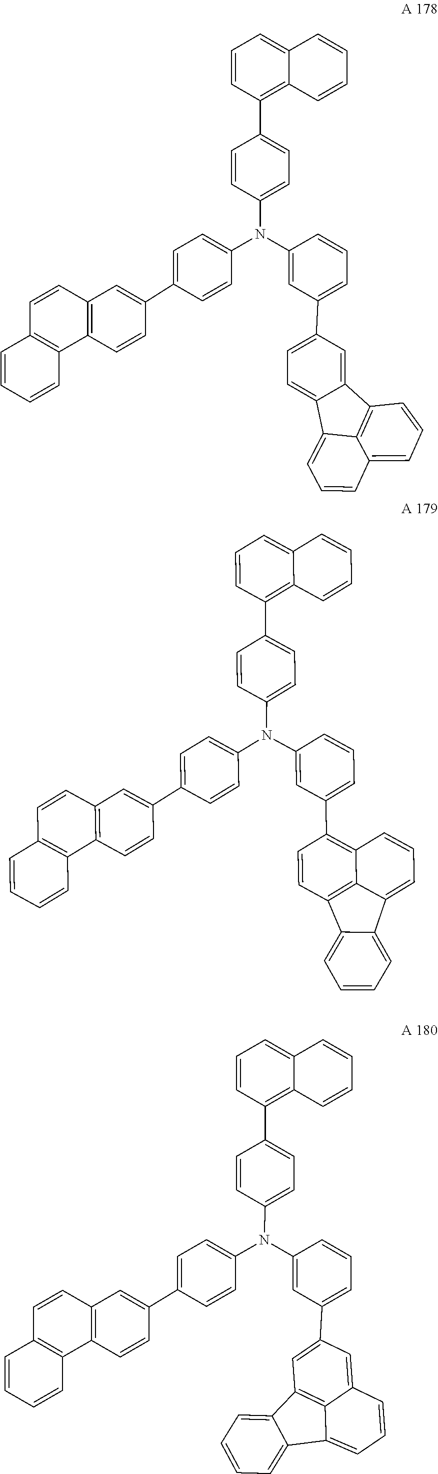

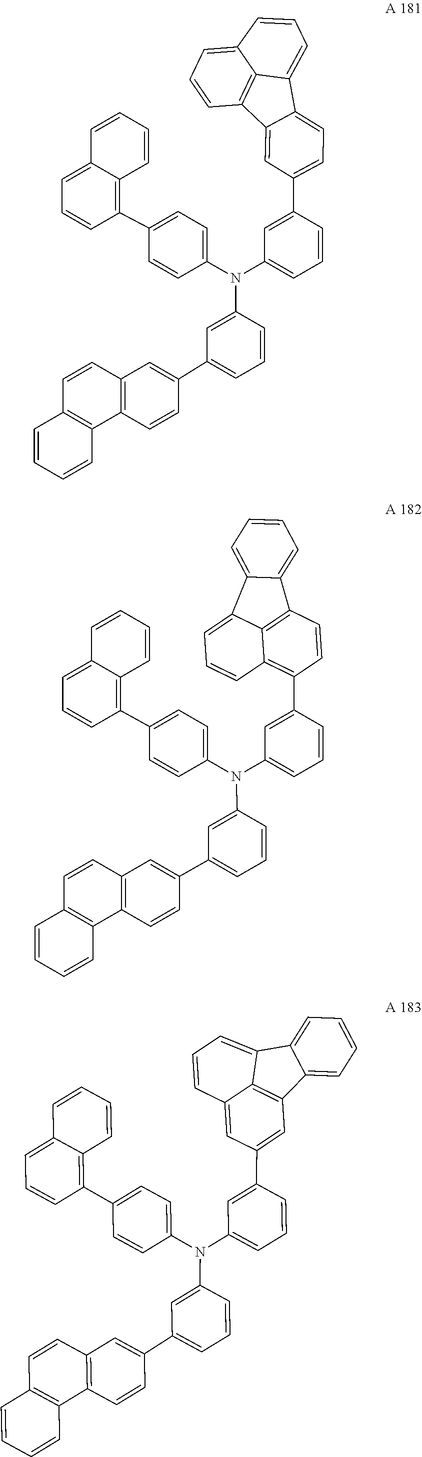 [SMILES: c1cc(-c2ccc3c(c2)-c2cccc4cccc-3c24)cc(N(c2ccc(-c3cccc4ccccc34)cc2)c2cccc(-c3ccc4c(ccc5ccccc54)c3)c2)c1 ; c1cc(-c2ccc3c(ccc4ccccc43)c2)cc(N(c2ccc(-c3cccc4ccccc34)cc2)c2cccc(-c3ccc4c5c(cccc35)-c3ccccc3-4)c2)c1 ; c1cc(-c2cc3c4c(cccc4c2)-c2ccccc2-3)cc(N(c2ccc(-c3cccc4ccccc34)cc2)c2cccc(-c3ccc4c(ccc5ccccc54)c3)c2)c1]

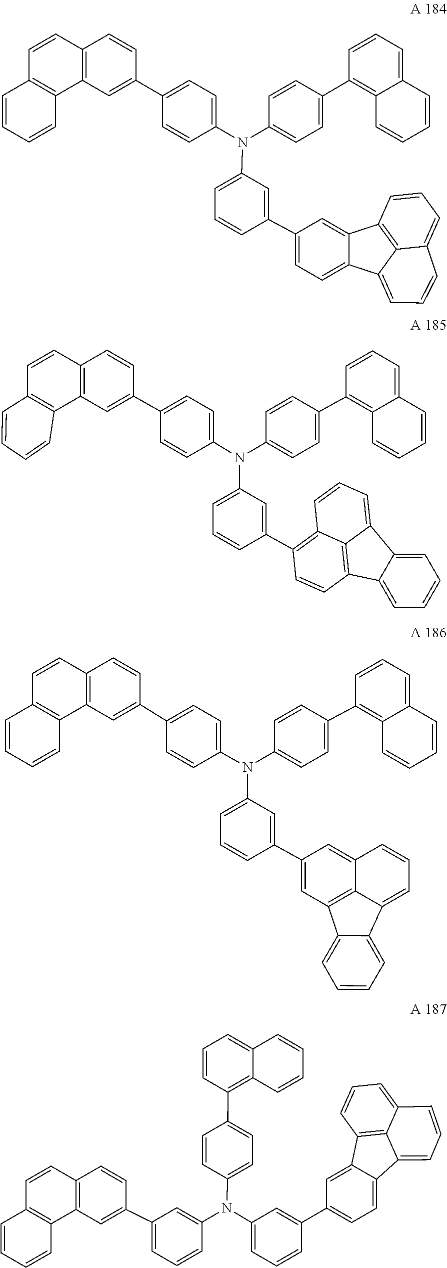 [SMILES: c1cc(-c2ccc3c(c2)-c2cccc4cccc-3c24)cc(N(c2ccc(-c3ccc4ccc5ccccc5c4c3)cc2)c2ccc(-c3cccc4ccccc34)cc2)c1 ; c1cc(-c2ccc3c4c(cccc24)-c2ccccc2-3)cc(N(c2ccc(-c3ccc4ccc5ccccc5c4c3)cc2)c2ccc(-c3cccc4ccccc34)cc2)c1 ; c1cc(-c2cc3c4c(cccc4c2)-c2ccccc2-3)cc(N(c2ccc(-c3ccc4ccc5ccccc5c4c3)cc2)c2ccc(-c3cccc4ccccc34)cc2)c1 ; c1cc(-c2ccc3c(c2)-c2cccc4cccc-3c24)cc(N(c2ccc(-c3cccc4ccccc34)cc2)c2cccc(-c3ccc4ccc5ccccc5c4c3)c2)c1]

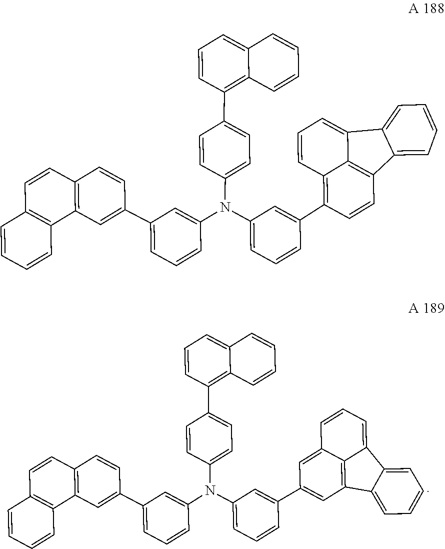 [SMILES: c1cc(-c2ccc3ccc4ccccc4c3c2)cc(N(c2ccc(-c3cccc4ccccc34)cc2)c2cccc(-c3ccc4c5c(cccc35)-c3ccccc3-4)c2)c1 ; c1cc(-c2cc3c4c(cccc4c2)-c2ccccc2-3)cc(N(c2ccc(-c3cccc4ccccc34)cc2)c2cccc(-c3ccc4ccc5ccccc5c4c3)c2)c1]

9. An electroluminescent device including the compound according to any one of claims 1 to 8.

10. The electroluminescent device according to claim 9, wherein the compound according to any one of claims 1 to 8 is used as a hole transport layer material or an electron blocking layer material.
